SEQUENCE LISTING (5 sequences)

SEQ ID NO: 1 - protein, 25 aa, synthetic construct
KKKKKKKKKKQWQWQWQWQWQWEEE

SEQ ID NO: 2 - protein, 9 aa, synthetic construct
KKKKKKKKK

SEQ ID NO: 3 - protein, 10 aa, synthetic construct
KKKKKKKKKK

SEQ ID NO: 4 - protein, 32 aa, synthetic construct
  VARIANT 1..15: note = Up to 7 copies of K may be deleted
  VARIANT 28..32: note = Up to 4 copies of E may be deleted
KKKKKKKKKKKKKKKQWQWQWQWQWQWEEEEE

SEQ ID NO: 5 - protein, 26 aa, synthetic construct
KKKKKKKKKAAAAAQWQWQWQWQWQW

(19) United States
(12) Patent Application Publication
(10) Pub. No.: US 2026/0102503 A1
(43) Pub. Date: Apr. 16, 2026

(54) NANOCOMPOSITES FOR ENHANCED CELLULAR PAYLOAD DELIVERY

(71) Applicant: Board of Regents, The University of Texas System, Austin, TX (US)

(72) Inventors: Kytai NGUYEN, Grand Prairie, TX (US); He DONG, Coppell, TX (US); Su YANG, Cary, NC (US); Uday CHINTAPULA, Norristown, PA (US)

(21) Appl. No.: 19/115,088

(22) PCT Filed: Oct. 4, 2023
(86) PCT No.: PCT/US2023/075900

§ 371 Date: Mar. 25, 2025

Related U.S. Application Data
(60) Provisional application No. 63/413,102, filed on Oct. 4, 2022.

(51) Int. Cl. A61K 47/62; A61K 47/69; B82Y 30/00

(52) U.S. Cl. CPC A61K 47/62; A61K 47/6937; B82Y 30/00

(57) ABSTRACT

Generally, the present disclosure is directed to compositions and methods of using the same. In some embodiments, a composition described herein comprises a nanoparticle, a plurality of nanofibers disposed on an exterior surface of the nanoparticle, and a payload disposed within an interior of the nanoparticle. The nanoparticle has an average size in three dimensions, and the plurality of nanofibers has an average length in a long dimension. In some cases, a ratio of the average size of the nanoparticle to the average length of the nanofibers is between 2 and 250.

### CROSS-REFERENCE TO RELATED APPLICATIONS

This application claims priority pursuant to 35 U.S.C. § 119 to U.S. Provisional Patent Application No. 63/413,102, filed Oct. 4, 2022, which is hereby incorporated by reference in its entirety.

### SEQUENCE LISTING

An electronic sequence listing (UTA 22-17PCT1.xml; size 5.95 kB; date of creation Sep. 28, 2023) submitted herewith is incorporated by reference in its entirety.

### FIELD

The present disclosure is directed to compositions and methods for treating and/or diagnosing a condition or disease in a patient in need thereof. In particular, nanocomposites for enhanced cellular delivery of payloads are described.

### BACKGROUND

Lung infections, especially lower respiratory tract infections and their associated pneumonia, are one of the leading causes of death, accounting for more than 4 million fatalities every year worldwide. Existing therapeutic formulations for inhalation pulmonary delivery suffer from one or more disadvantages. For example, some formulations cannot readily reach the lower respiratory tract owing to low cell uptake and retention during infection conditions. Failure to penetrate mucosal layers or degeneration within mucus are additional weaknesses of some existing formulations. More generally, limitations of existing drug delivery strategies for treating bacterial infections in the lungs or elsewhere can further include low compliance in older patients and side effects of antibiotics including acute kidney injury, cytotoxicity, and nephrotoxicity b off-targeting. There is thus a need for improved formulations for treating and/or diagnosing infections or other conditions in patients, including pulmonary conditions.

### SUMMARY

Generally, the present application is directed to compositions and methods for treating and/or diagnosing a disease or condition. More particularly, the present application is directed to nanocomposite compositions and their use for treating and/or diagnosing a condition or disease in a patient or other subject. For instance, in one aspect, methods of treating and/or diagnosing a human patient in need thereof comprise providing a composition described herein to the patient having a lung or respiratory disease or condition.

In some embodiments, a composition described herein comprises a nanoparticle, a plurality of nanofibers disposed on an exterior surface of the nanoparticle, and a payload disposed within an interior of the nanoparticle. In some implementations, the nanoparticle has an average size in three dimensions, the plurality of nanofibers has an average length in a long dimension, and a ratio of the average size of the nanoparticle to the average length of the nanofibers is between 2 and 250. In some embodiments, a ratio of the average size of the nanoparticle to the average length of the nanofibers is between 5 and 100. In other embodiments, a ratio of the average size of the nanoparticle to the average length of the nanofibers is between 5 and 30. Further, in some instances, the nanoparticle has an average surface area, the plurality of nanofibers has an average length in a long dimension, and a ratio of the average surface area of the nanoparticle to the average length of the nanofibers, in units of (μm), is between 0.6 and 4,000. Additionally, in some cases, the average size of the nanoparticle in three dimensions is between 0.1 μm and 5 μm, and the average length of the nanofibers in the long dimension is between 20 nm and 50 nm. In some embodiments, the average width of the nanofibers in one or two dimensions is less than 10 nm.

Moreover, in some instances, the nanofibers of a composition described herein are present in the composition in an amount of 0.5 to 15 wt. %, based on the total weight of the composition. Additionally, in some cases, the payload is present in the composition in an amount of 1-80 wt. %, based on the total weight of the composition. The nanoparticle component, in some implementations, is present in an amount of 10-80 wt. %.

Further, in some embodiments, the exterior surface of the nanoparticle of a composition described herein has an opposite charge compared to a solvent-facing charge density of the plurality of nanofibers, where it is understood that a “solvent-facing” side, direction, or charge refers to the side or part or charge of nanofibers (or other component) that is closest to or in contact with solvent or other exterior environment, such as that surrounding a nanocomposite described herein, when disposed within a biological compartment or within a patient. In some cases, the exterior surface of the nanoparticle is negatively charged or has a negative zeta potential, and the nanofibers have a positive solvent-facing charge density or a positive zeta potential.

In addition, in some implementations, the nanoparticle of a composition described herein is formed from a biocompatible and/or biodegradable material. In some cases, the nanoparticle comprises a lipid nanoparticle or a liposome. In other instances, the nanoparticle is formed from an inorganic material. The nanoparticle may also be formed from an organic material such an organic polymer, as described further below.

Further, in some embodiments, a nanoparticle of a composition described herein is porous. Moreover, in some cases, the nanofibers of the composition comprise polypeptide nanofibers, such as self-assembled polypeptide nanofibers or multidomain peptides (MDPs).

The payload of a composition described herein is not particularly limited. In some implementations, for example, the payload comprises an imaging agent, a therapeutic agent, a theranostic agent, or a combination of two or more of the foregoing. In other instances, the payload comprises nucleic acids, proteins, peptides, chemotherapeutics, vaccine components, antibiotics, or a combination of two or more of the foregoing. The payload can be physically entrapped within the interior of the nanoparticle and can be operable to diffuse out of the interior of the nanoparticle when the composition is disposed in an aqueous or biological environment, as described further hereinbelow.

A method described of treating and/or diagnosing a condition or disease in a patient in need thereof, in some embodiments, comprises disposing a composition described herein within a biological compartment of the patient, such as lungs or another pulmonary site of the patient. Further, in some cases, a method described herein comprises penetrating a membrane of a cell or population of cells within the biological compartment with the plurality of nanofibers of the composition, and subsequently releasing at least a portion of the payload of the composition within a cytosol of the cell or population of cells after penetrating the membrane of the cell or population of cells. Additionally, in some embodiments, a method described herein further comprises biologically degrading the nanoparticle and/or the plurality of nanofibers of the composition after penetrating the membrane of the cell or population of cells. Biodegraded components may also be cleared from the patient following degradation.

Further, in some implementations of a method described herein, the payload of the composition comprises an imaging agent or a theranostic agent, and the method further comprises imaging the cell or population of cells with the imaging agent or theranostic agent. Moreover, in some cases, a composition described herein is disposed within the biological compartment of the patient by inhalation or nebulization. In addition, in some preferred embodiments of methods described herein, the treated and/or diagnosed condition or disease comprises a respiratory condition or disease. Further, in some such cases, the biological compartment (to which the composition is delivered) is a pulmonary site. Moreover, in some embodiments described herein, a treated and/or diagnosed respiratory condition or disease is caused by a pathogen or product of a pathogen, and the payload comprises a therapeutic agent effective for the treatment of the condition or disease caused by the pathogen or product of the pathogen.

The foregoing embodiments and other embodiments are further described in the detailed description which follows.

### BRIEF DESCRIPTION OF THE FIGURES

FIG. 1 schematically illustrates nanocomposite synthesis according to one embodiment described herein.

FIG. 2A is the MALDI-TOF spectrum of a peptide with a primary sequence of K₁₀(QW)₆E₃ (SEQ ID NO: 1), according to some embodiment described herein.

FIG. 2B is the MALDI-TOF spectrum of a peptide with a primary sequence of K₁₀(QW)₆E₃ (SEQ ID NO: 1) labeled with fluorescein isothiocyanate (FITC), according to some embodiment described herein.

FIG. 3A is a bar graph of the zeta potential and particle size of nanoparticles, nanocomposites, and nanofibers from dynamic light scattering measurements, according to some embodiments described herein.

FIG. 3B displays a TEM image of nanocomposites with white arrows pointing to the nanofibers attached to the nanoparticles, according to one embodiment described herein. The scale bar is 50 μm.

FIG. 3C shows fluorescent images of nanoparticles loaded with rhodamine B dye and nanocomposites of nanoparticles loaded with rhodamine B dye coated with FITC-labeled nanofibers, according to some embodiments described herein. The scale bar is 20 μm.

FIG. 3D is a plot of the nanofiber concentration compared to the Fₙₒᵣₘ value (permille) of rhodamine B-labeled nanocomposites at a fixed concentration of 2 mg/mL, according to some embodiments described herein.

FIG. 3E displays the FTIR spectra of nanofiber-coated poly(lactic-co-glycolic acid) (PLGA) nanoparticles, plain PLGA nanoparticles, and nanofibers alone, according to some embodiments described herein.

FIG. 4A is a raw cryo-EM image of nanoparticles coated with nanofibers, which are indicated as black arrows, according to some embodiments described herein.

FIG. 4B is a contrast-enhanced cryo-EM image of nanoparticles coated with nanofibers, which are indicated as black arrows, according to some embodiments described herein.

FIG. 5 is a plot of the capillary positions related to the raw fluorescence of empty capillaries, according to some embodiments described herein.

FIG. 6 is a bar graph of the cell viability of AT1 cells normalized to untreated cells for nanofibers alone, nanoparticles alone, and nanocomposites at 0.0625, 0.125, 0.25, 0.5, and 1 mg/mL, according to some embodiments described herein. Cell viability was assessed using a 3-(4,5-dimethylthiazol-2-yl)-5-(3-carboxymethoxyphenyl)-2-(4-sulfophenyl)-2H-tetrazolium (MTS) assay. Asterisks indicate *p<0.0001 according to a two-way ANOVA with Sidak's multiple comparisons test.

FIG. 7A is a bar graph of the uptake of nanoparticles with and without nanofibers by overnight cultured primary lung AT1 cells, according to some embodiments described herein. Asterisks indicate **p<0.01 and ***p<0.001.

FIG. 7B is fluorescent images of AT1 cells treated with nanoparticles with and without nanofibers after 90 minutes of treatment, washing 3 times with PBS, and staining with DAPI, according to one embodiment described herein. The scale bar is 20 μm.

FIG. 7C is a time course plot of the mean fluorescent intensity of nanoparticles or nanocomposites applied to AT1 cells for 30 minutes, 90 minutes, and 4 hours, according to some embodiments described herein.

FIG. 7D is the 3D reconstruction of confocal images of AT1 cells exposed to nanoparticles or nanocomposites, according to some embodiment described herein. The white boxes indicate x-z and y-z slices. The scale bar x-y is 50 μm, the x-z scale bar is 20 μm, and the y-z scale bar is 20 μm.

FIG. 8A is a bar graph of the mean fluorescent intensity of AT1 cells at confluency treated with nanocomposites loaded with rhodamine B at 31.5, 62.5, 125, 250, and 500 μg/mL for 90 minutes compared to a nanoparticle control at 500 μg/mL, according to one embodiment described herein. Asterisks indicate **p<0.01.

FIG. 8B is fluorescence microscopy images of AT1 cells at confluency treated with 31.5, 62.5, 125, 250, or 500 μg/mL of nanocomposites loaded with rhodamine B or 500 μg/mL of a nanoparticle control, according to some embodiments described herein.

FIG. 9A is a bar graph of the uptake of nanoparticles or nanocomposites at 0.125, 0.25, and 0.5 mg/mL by RAW macrophage cells, according to some embodiment described herein. Asterisks indicate **p<0.01 and ***p<0.001 according to a two-way ANOVA analysis was done with Sidak's multiple comparison test.

FIG. 9B is a bar graph of the uptake of nanoparticles or nanocomposites at 0.125, 0.25, and 0.5 mg/mL by human umbilical vein endothelial cells (HUVECs), according to some embodiment described herein. Asterisks indicate *p<0.01 and ****p<0.0001 according to a two-way ANOVA analysis was done with Sidak's multiple comparison test.

FIG. 9C is fluorescent microscopy images of RAW macrophage cells and HUVECs treated with nanoparticles only or nanocomposites, according to some embodiments described herein.

FIG. 9D is additional fluorescent microscopy images of RAW macrophage cells treated with nanoparticles only or nanocomposites, according to some embodiments described herein.

FIG. 10 is a bar graph of the cell viability of HUVECs treated with nanocomposites at 50, 100, 250, 500, and 1000 μg/mL for 72 hours or 500 μg/mL of a nanoparticle control, according to some embodiments described herein. Asterisks indicate **p<0.01 and ****p<0.0001 according to a one way ANOVA with Dunnett's multiple comparison test.

FIG. 11A is a time course of fluorescent microscopy and bright field images of AT1 cells treated with nanocomposites labeled with rhodamine B and coated with FITC-labeled nanofibers for 2 hours, 8 hours, and 24 hours and then stained with LysoTracker™ Blue DND-22, according to some embodiments described herein.

FIG. 11B is a merged image of LysoTracker™ Blue DND-22, FITC, and rhodamine B from the time course in FIG. 11A at 8 hours, according to some embodiments described herein.

FIG. 11C is a plot of the fluorescent intensity of of LysoTracker™ Blue DND-22, FITC, and rhodamine B at 8 hours, according to some embodiments described herein. The x axis is length in arbitrary units, and the y axis is the fluorescent intensity in the region of interest and overlap of fluorescence in arbitrary units.

FIG. 11D is a merged image of LysoTracker™ Blue DND-22, FITC, and rhodamine B from the time course in FIG. 11A at 24 hours according to some embodiments described herein.

FIG. 11E is a plot of the fluorescent intensity of of LysoTracker™ Blue DND-22, FITC, and rhodamine B at 24 hours according to some embodiments described herein. The x axis is length in arbitrary units, and the y axis is the fluorescent intensity in the region of interest and overlap of fluorescence in arbitrary units.

FIG. 12A is a bar graph of the mean fluorescent intensity of AT1 cells treated with nanocomposites for 90 minutes after treating cells with various endocytosis inhibitors for 2 hours, according to some embodiments described herein. Multiple comparisons were done using Holm-Sidak's multiple comparison test. Asterisks indicate *p<0.05 and **p<0.01.

FIG. 12B is fluorescent microscopy images of AT1 cells treated with nanocomposites for 90 minutes after treating cells with various endocytosis inhibitors for 2 hours, according to some embodiments described herein. The scale bar is 90 μm.

FIG. 12C is fluorescent microscopy images of AT1 cells treated with nanocomposites for 90 minutes after treating cells with the endocytosis inhibitors Dynasore, Imipramine, and Cytochalasine-D for 2 hours as compared to untreated cells, according to some embodiments described herein.

FIG. 12D is a bar graph of the uptake of nanoparticles or nanocomposites at 0.5 mg/mL in AT1 cells precooled at 4° C. for 30 minutes before treatment with nanoparticles or nanocomposites and 37° C., according to one embodiment described herein. Asterisks indicate ****p<0.0001.

FIG. 12E is fluorescent microscopy images of nanoparticles and nanocomposites at 4° C. and 37° C. from FIG. 12D herein, according to one embodiment described herein.

FIG. 12F is fluorescent microscopy images of the uptake of nanoparticles or nanocomposites by AT1 cells precooled at 4° C. for 90 minutes before treatment with nanoparticles or nanocomposites and 37° C., according to one embodiment described herein.

FIG. 13A is a schematic of the mucus permeation study, according one embodiment described herein.

FIG. 13B is a time course plot over 60 hours of the concentration of the nanoparticles and nanocomposites migrated through the simulated mucus of the mucus permeation study shown in FIG. 13A, according to one embodiment described herein.

FIG. 13C is the first 10 hours of the timecourse plot shown in FIG. 13B, according to one embodiment described herein. Asterisks indicate *p<0.05, and **p<0.01, and ****p<0.0001.

FIG. 14A is an image of the nebulizer and an attached 3D printed extension to deliver nanoparticles or nanocomposites to cells in a 12-well plate, according to some embodiments described herein.

FIG. 14B is a bar graph of the percent uptake after the delivery of nanoparticles and nanocomposites via nebulization to AT1 cells, according to one embodiment described herein. Asterisks indicate *p<0.05 using a paired t-test.

FIG. 14C is a bar graph of the uptake of nanoparticles and nanocomposites after the delivery of nanoparticles and nanocomposites via nebulization to AT1 cells normalized to total cell protein, according to one embodiment described herein. Asterisks indicate ***p<0.001.

FIG. 14D is fluorescence microscopy images of AT1 cells showing the uptake of nanoparticles and nanocomposites after nebulization, according to one embodiment described herein. The scale bar is 50 μm.

FIG. 14E is a bar graph of the uptake of nanoparticles and nanocomposites before and after freeze drying normalized to total cell protein, according to some embodiments described herein. Two-way ANOVA with Sidak's multiple comparison was done with ****p<0.0001.

FIG. 14F is a bar graph of the uptake of nanocomposities and HIV Tat peptide coated nanoparticles normalized to total cell protein, according to some embodiments described herein. Asterisk indicates *p<0.05 according to paired t-test.

FIG. 15A is a bar graph of the zeta potential of synthesized nanoparticles from dynamic light scattering measurements according to some embodiments described herein. Asterisks indicate **p<0.01.

FIG. 15B is a bar graph of the uptake of nanoparticles at the indicated concentrations normalized to total cell protein. Asterisks indicate *p<0.05, ***p<0.001, and ****p<0.0001.

FIG. 16 is a schematic of an antimicrobial nanocomposite (AMNC), according to an embodiment described herein.

FIG. 17 is a schematic of the delivery of an AMNC to the lungs for the treatment of S. aureus, specifically MRSA, according to an embodiment described herein.

FIG. 18 schematically illustrates AMNC synthesis with a vancomycin-dextran sulfate payload according to one embodiment described herein.

FIG. 19A is a transmission electron microscope image of AMNCs loaded with vancomycin according to one embodiment described herein. The scale bar is 50 nm.

FIG. 19B is a plot of the drug release profile of vancomycin from PLGA nanoparticles with vancomycin bound and PLGA nanoparticles with vancomycin bound coated in nanofibers according to some embodiments described herein.

FIG. 19C is a bar graph of the percent change in size of AMNCs in saline over 72 hours according to some embodiments described herein.

FIG. 20A is a bar graph of the cell viability of AT1 cells as a percentage of untreated cells treated with AMNCs at 50, 100, 250, 500, and 100 μg/mL, according to some embodiments described herein. Asterisks indicate ****p<0.0001.

FIG. 20B is a bar graph of the cell viability of A459 cells as a percentage of untreated cells treated with AMNCs at 50, 100, 250, 500, and 100 μg/mL, according to some embodiments described herein.

FIG. 21A is flow cytometry results for AMNC uptake in MRSA-infected AT1 cells after 90 minutes of treatement, according to some embodiments described herein. MRSA was stained with SYTO 9 gated on the y-axis, and AMNCs were stained with rhodamine B gated on the x-axis.

FIG. 21B show representative fluorescent images of nanoparticles and nanocomposites, according to some embodiments described herein.

FIG. 21C is a bar graph of the uptake of nanoparticles and nanocomposites by AT1 cells infected with MRS at MOIs of 1:0.5, 1:1, 1:10, and 1:100, according to some embodiments described herein. Asterisks indicate *p<0.05, **p<0.01, ***p<0.001, and ****p<0.0001.

FIG. 22A is a bar graph of the OD at 600 nm of samples of MRSA treated with the indicated concentrations of PLGA-vancomycin and stained with 0.015% resazurin to assess bacterial growth, according to some embodiments described herein. The asterisk indicates *p<0.05.

FIG. 22B is an image of a plate of MRSA cultures treated with the indicated controls and concentrations of PLGA-vancomycin and stained with 0.015% resazurin to assess bacterial growth, according to some embodiments described herein.

FIG. 22C is an image of the agar plate of the results of the zone of inhibition study in which MRSA was plated on BHI agar and sterile discs were loaded with (1) only BHI media, (2) plain NPs, (3) 2X MIC of free vancomycin, (4) 1X MIC of free vancomycin, (5) 2X MIC of vancomycin-loaded nanoparticles, and (6) 1X MIC of vancomycin-loaded nanoparticles, according to some embodiments herein.

FIG. 22D is a bar graph of the inhibition diameter for each condition in FIG. 22C as measured using the ImageJ software, according to some embodiments herein.

FIG. 23A is a bar graph of the plated intracellular bacteria of AT1 cells treated with bacteria only, free vancomycin, vancomycin-loaded PLGA nanoparticles (PLGA-NPs), or AMNCs for 90 minutes, according to some embodiments herein. Asterisks indicate *p<0.05 and **p<0.01.

FIG. 23B is an image of the plated intracellular bacteria of AT1 cells treated with bacteria only, free vancomycin, vancomycin-loaded PLGA NPs, or AMNCs for 90 minutes, according to some embodiments herein. Asterisks indicate *p<0.05 and **p<0.01.

FIG. 24A is a bar graph of the plated intracellular bacteria of AT1 cells treated with vancomycin-loaded PLGA-NPs or AMNCs for 90 minutes or left untreated, according to some embodiments herein.

FIG. 24B is an image of the plated intracellular bacteria of AT1 cells treated with vancomycin-loaded PLGA-NPs or AMNCs for 90 minutes or left untreated, according to some embodiments herein.

FIG. 25A is a schematic of the inhalation delivery system of nebulized PLGA-NPs and AMNCs in mice, according to some embodiments described herein.

FIG. 25B is an image of the inhalation delivery system of nebulized PLGA-NPs and AMNCs in mice, according to some embodiments described herein.

FIG. 26A is a bar graph of the percentage of ICG-loaded PLGA-NPs and AMNCs that were delivered to the lungs via nebulization in mice that received nebulized treatment, according to some embodiments described herein.

FIG. 26B is a bar graph of the amount of ICG-loaded PLGA-NPs and AMNCs that were delivered to the lungs via nebulization in mice that received nebulized treatment per mg of lung tissue, according to some embodiments described herein.

FIG. 26C is images of hematoxylin and eosin staining of paraffin-embedded lung tissue samples from mice that received nebulized treatment of PLGA-NPs and AMNCs, according to some embodiments described herein.

FIG. 27A is fluorescent images of lung tissue sections from mice treated with nebulization treatments of saline or AMNCs loaded with coumarin-6 dye stained with DAPI, according to some embodiments herein.

FIG. 27B shows fluorescent images of lung tissue from mice treated with nebulization treatments of saline, nanoparticles, or AMNCs, according to some embodiments herein.

FIG. 28 is a schematic of the uptake and delivery of PLGA remdesivir-loaded PLGA nanoparticles into lung epithelial cells to inhibit SARS-CoV-2, according to some embodiments herein.

FIG. 29A is a transmission electron microscope image of remdesivir-loaded PLGA nanoparticles, according to some embodiments herein. The scale bar is 100 nm.

FIG. 29B is fluorescent microscopy images of remdesivir-loaded and rhodamine B-loaded PLGA nanoparticles coated in FITC-labelled nanofibers, according to some embodiments herein. The scale bar is 460 nm.

FIG. 29C is a plot of the drug release profile of remdesivir from remdesivir-loaded PLGA nanoparticles, according to some embodiments described herein.

FIG. 29D is a bar graph of the cell viability of AT1 cells and Vero E₆ cells treated with AMNCs at 10, 50, 100, 250, 500, and 1000 μg/mL normalized to untreated cells according to some embodiments herein.

FIG. 30A shows fluorescent images of Vero E6 cells treated with 0, 50, and 100 μg/mL of nanoparticles loaded with rhodamine B and stained with DAPI, according to some embodiments herein.

FIG. 30B shows fluorescent images of Vero E6 cells treated with 0, 50, and 100 μg/mL of nanocomposites loaded with rhodamine B and stained with DAPI, according to some embodiments herein.

FIG. 31A is a bar graph of the RT-qPCR results for the ratio of nCoV-N1 to β-actin in Vero E6 cells infected with SARS-CoV2 and left untreated, treated with blank nanoparticles, treated with remdesivir, and treated with nanoparticles loaded with remdesivir at 10, 100, and 1000 μg/mL, according to some embodiments herein. Asterisks indicate *p<0.05, **p<0.01, ***p<0.001, and ****p<0.0001.

FIG. 31B is a bar graph of the RT-qPCR results for the ratio of nCoV-N1 to β-actin in Vero E6 cells infected with SARS-CoV2 and left untreated, treated with blank nanoparticles, treated with remdesivir, treated with nanoparticles loaded with remdesivir, and treated with nanocomposites loaded with remdesivir for 24 hours, according to some embodiments herein. Asterisks indicate *p<0.05, **p<0.01, ***p<0.001, and ****p<0.0001.

FIG. 31C is a bar graph of the RT-qPCR results for the ratio of nCoV-N1 to β-actin in Vero E6 cells infected with SARS-CoV2 and left untreated, treated with blank nanoparticles, treated with remdesivir, treated with nanoparticles loaded with remdesivir, and treated with nanocomposites loaded with remdesivir for 48 hours, according to some embodiments herein. Asterisks indicate *p<0.05, **p<0.01, ***p<0.001, and ****p<0.0001.

FIG. 31D is confocal images of Vero E6 cells infected with SARS-CoV2 and left untreated, treated with remdesivir, treated with blank nanoparticles, treated with nanoparticles loaded with remdesivir, treated with nanocomposites loaded with remdesivir, and treated with ritonavir, according to some embodiments herein.

FIG. 31E is a bar graph of the viral titer of supernatants collected from Vero E6 cells pre-infected with SARS-CoV-2 after 24 hours left untreated, treated with remdesivir, treated with blank nanoparticles (plain nanoparticles without any drug loaded), nanoparticles loaded with remdesivir, and nanocomposites loaded with remdesivir, according to some embodiments herein.

FIG. 31F shows images from the plates from the plaque assay from the supernatants of Vero E6 cells treated with remdesivir, treated with nanoparticles loaded with remdesivir, treated with nanocomposites loaded with remdesivir, and blank nanoparticles, according to some embodiments herein.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

It is also to be understood that the article “a” or “an” refers to “at least one,” unless the context of a particular use requires otherwise.

In general, the present disclosure is directed to compositions such as nanocomposites, and methods of making and using such compositions, including for the diagnosis and/or treatment of a condition or disease in a patient in need thereof. In one aspect, compositions are particularly described herein. In some embodiments, a composition comprises a nanoparticle, a plurality of nanofibers disposed on an exterior surface of the nanoparticle, and a payload disposed within an interior of the nanoparticle.

Further, in some embodiments, the nanoparticle has an average size in three dimensions and the plurality of nanofibers has an average length in a long dimension. For reference purposes herein, the “average size in three dimensions” of a nanoparticle described herein is the diameter of a spherical nanoparticle having an equal volume to the nanoparticle of the composition. In some cases, a nanoparticle described herein is round or spherical, or substantially spherical. In such instances, the “average size in three dimensions” is equal to the diameter of the nanoparticle. However, a nanoparticle described herein can also have a non-spherical shape. In general, a nanoparticle can have any shape not inconsistent with the technical objectives of the present disclosure. For example, in some embodiments, a nanoparticle described herein is oblate or has an aspect ratio other than 1, where aspect ratio is defined as the ratio of the length to the width of a particle. For example, in some cases, the aspect ratio can be greater than 1.1 or 1.2, or between 1.1 and 1.5. In some embodiments, a nanoparticle described herein can have a cylindrical or rod shape, a regular polyhedral shape such as a cube shape, an irregular polyhedral shape, or another regular or irregular shape.

Therefore, it is to be understood that the “average size in three dimensions” of a non-spherical nanoparticle described herein is equal to and considered to be the same as the diameter of a spherical nanoparticle having an equal volume to the non-spherical nanoparticle of the composition. For instance, as one non-limiting example, if the nanoparticle is a cube, the volume of the cube is s³, wherein s is the length of one side of the cube. Such a cubed-shaped nanoparticle, for reference purposes herein, is treated as having a “average size in three dimensions” equal to the diameter of a sphere that has a volume of s³. Thus, in this instance, it can be derived. This volume is equivalent to:

$$V = \frac{1}{6} π {d}^{3} , \qquad ( 1 )$$

wherein d is the diameter of the equivalent sphere. In this non-limiting example, the diameter of the equivalent sphere is:

$$d = \sqrt[3]{\frac{1}{6} π {s}^{3}} \qquad ( 2 )$$

In other instances, wherein the nanoparticles are irregularly shaped, the average size of nanoparticles with irregular shapes with known volumes can be determined by equating the volume of the irregular nanoparticle to the volume of a sphere (Formula 1) and determining d, the diameter of the equivalent sphere.

Moreover, in some cases, a ratio of the average size of the nanoparticle to the average length of the nanofibers is between 2 and 250. In some instances, the ratio of the average size of the nanoparticle to the average length of the nanofibers is between 5 and 100. In other embodiments, the ratio of the average size of the nanoparticle to the average length of the nanofibers is between 5 and 30. In some cases, the ratio is between 2 and 20, between 2 and 40, between 2 and 60, between 2 and 80, between 2 and 100, between 2 and 120, between 2 and 140, between 2 and 160, between 2 and 180, between 2 and 200, between 2 and 220, between 5 and 20, between 5 and 20, between 5 and 40, between 5 and 60, between 5 and 80, between 5 and 100, between 5 and 120, between 5 and 140, between 5 and 160, between 5 and 180, between 5 and 200, between 5 and 220, between 10 and 40, between 10 and 60, between 10 and 80, between 10 and 100, between 10 and 120, between 10 and 140, between 10 and 160, between 10 and 180, between 10 and 200, between 10 and 220, between 20 and 40, between 20 and 60, between 20 and 80, between 20 and 100, between 20 and 120, between 20 and 140, between 20 and 160, between 20 and 180, between 20 and 200, between 20 and 220, between 40 and 60, between 40 and 80, between 40 and 100, between 40 and 120, between 40 and 140, between 40 and 160, between 40 and 180, between 40 and 200, between 40 and 220, between 60 and 80, between 60 and 100, between 60 and 120, between 60 and 140, between 60 and 160, between 60 and 180, between 60 and 200, between 60 and 220, between 80 and 100, between 80 and 120, between 80 and 140, between 80 and 160, between 80 and 180, between 80 and 200, between 80 and 220, between 100 and 120, between 100 and 140, between 100 and 160, between 100 and 180, between 100 and 200, between 100 and 220, between 120 and 140, between 120 and 160, between 120 and 180, between 120 and 200, between 120 and 220, between 140 and 160, between 140 and 180, between 140 and 200, between 140 and 220, between 160 and 180, between 160 and 200, between 160 and 220, between 180 and 200, between 180 and 220, between 200 and 220, or between 220 and 250.

Further, in some embodiments, the nanoparticle has an average surface area, and the plurality of nanofibers has an average length in a long dimension. In some cases, a ratio of the average surface area of the nanoparticle to the average length of the nanofibers, in units of (μm), is between 0.6 and 4,000. It is to be understood that the average surface area of the nanoparticle can be considered as the surface area of a nanoparticle with the diameter of a spherical nanoparticle having an equal volume of the nanoparticle of the composition. In some instances, the ratio of the average surface area of the nanoparticle to the average length of the nanofibers, in units of (μm), is between 0.6 and 5, between 0.6 and 10, between 0.6 and 20, between 0.6 and 40, between 0.6 and 50, between 0.6 and 75, between 0.6 and 100, between 0.6 and 200, between 0.6 and 300, between 0.6 and 400, between 0.6 and 500, between 0.6 and 600, between 0.6 and 700, between 0.6 and 800, between 0.6 and 900, between 0.6 and 1,000, between 0.6 and 1,250, between 0.6 and 1,500, between 0.6 and 1,750, between 0.6 and 2,000, between 0.6 and 2,250, between 0.6 and 2,500, between 0.6 and 2,750, between 0.6 and 3,000, between 0.6 and 3,250, between 0.6 and 3,500, between 0.6 and 3,750, between 0.6 and 4,000, between 5 and 10, between 5 and 20, between 5 and 40, between 5 and 50, between 5 and 75, between 5 and 100, between 5 and 200, between 5 and 300, between 5 and 400, between 5 and 500, between 5 and 600, between 5 and 700, between 5 and 800, between 5 and 900, between 5 and 1,000, between 5 and 1,250, between 5 and 1,500, between 5 and 1,750, between 5 and 2,000, between 5 and 2,250, between 5 and 2,500, between 5 and 2,750, between 5 and 3,000, between 5 and 3,250, between 5 and 3,500, between 5 and 3,750, between 5 and 4,000, between 10 and 20, between 10 and 40, between 10 and 50, between 10 and 75, between 10 and 100, between 10 and 200, between 10 and 300, between 10 and 400, between 10 and 500, between 10 and 600, between 10 and 700, between 10 and 800, between 10 and 900, between 10 and 1,000, between 10 and 1,250, between 10 and 1,500, between 10 and 1,750, between 10 and 2,000, between 10 and 2,250, between 10 and 2,500, between 10 and 2,750, between 10 and 3,000, between 10 and 3,250, between 10 and 3,500, between 10 and 3,750, between 10 and 4,000, between 20 and 40, between 20 and 50, between 20 and 75, between 20 and 100, between 20 and 200, between 20 and 300, between 20 and 400, between 20 and 500, between 20 and 600, between 20 and 700, between 20 and 800, between 20 and 900, between 20 and 1,000, between 20 and 1,250, between 20 and 1,500, between 20 and 1,750, between 20 and 2,000, between 20 and 2,250, between 20 and 2,500, between 20 and 2,750, between 20 and 3,000, between 20 and 3,250, between 20 and 3,500, between 20 and 3,750, between 20 and 4,000, between 40 and 50, between 40 and 75, between 40 and 100, between 40 and 200, between 40 and 300, between 40 and 400, between 40 and 500, between 40 and 600, between 40 and 700, between 40 and 800, between 40 and 900, between 40 and 1,000, between 40 and 1,250, between 40 and 1,500, between 40 and 1,750, between 40 and 2,000, between 40 and 2,250, between 40 and 2,500, between 40 and 2,750, between 40 and 3,000, between 40 and 3,250, between 40 and 3,500, between 40 and 3,750, between 40 and 4,000, between 50 and 75, between 50 and 100, between 50 and 200, between 50 and 300, between 50 and 400, between 50 and 500, between 50 and 600, between 50 and 700, between 50 and 800, between 50 and 900, between 50 and 1,000, between 50 and 1,250, between 50 and 1,500, between 50 and 1,750, between 50 and 2,000, between 50 and 2,250, between 50 and 2,500, between 50 and 2,750, between 50 and 3,000, between 50 and 3,250, between 50 and 3,500, between 50 and 3,750, between 50 and 4,000, between 75 and 100, between 75 and 200, between 75 and 300, between 75 and 400, between 75 and 500, between 75 and 600, between 75 and 700, between 75 and 800, between 75 and 900, between 75 and 1,000, between 75 and 1,250, between 75 and 1,500, between 75 and 1,750, between 75 and 2,000, between 75 and 2,250, between 75 and 2,500, between 75 and 2,750, between 75 and 3,000, between 75 and 3,250, between 75 and 3,500, between 75 and 3,750, between 75 and 4,000, between 100 and 200, between 100 and 300, between 100 and 400, between 100 and 500, between 100 and 600, between 100 and 700, between 100 and 800, between 100 and 900, between 100 and 1,000, between 100 and 1,250, between 100 and 1,500, between 100 and 1,750, between 100 and 2,000, between 100 and 2,250, between 100 and 2,500, between 100 and 2,750, between 100 and 3,000, between 100 and 3,250, between 100 and 3,500, between 100 and 3,750, between 100 and 4,000, between 200 and 300, between 200 and 400, between 200 and 500, between 200 and 600, between 200 and 700, between 200 and 800, between 200 and 900, between 200 and 1,000, between 200 and 1,250, between 200 and 1,500, between 200 and 1,750, between 200 and 2,000, between 200 and 2,250, between 200 and 2,500, between 200 and 2,750, between 200 and 3,000, between 200 and 3,250, between 200 and 3,500, between 200 and 3,750, between 200 and 4,000, between 300 and 400, between 300 and 500, between 300 and 600, between 300 and 700, between 300 and 800, between 300 and 900, between 300 and 1,000, between 300 and 1,250, between 300 and 1,500, between 300 and 1,750, between 300 and 2,000, between 300 and 2,250, between 300 and 2,500, between 300 and 2,750, between 300 and 3,000, between 300 and 3,250, between 300 and 3,500, between 300 and 3,750, between 300 and 4,000, between 400 and 500, between 400 and 600, between 400 and 700, between 400 and 800, between 400 and 900, between 400 and 1,000, between 400 and 1,250, between 400 and 1,500, between 400 and 1,750, between 400 and 2,000, between 400 and 2,250, between 400 and 2,500, between 400 and 2,750, between 400 and 3,000, between 400 and 3,250, between 400 and 3,500, between 400 and 3,750, between 400 and 4,000, between 500 and 600, between 500 and 700, between 500 and 800, between 500 and 900, between 500 and 1,000, between 500 and 1,250, between 500 and 1,500, between 500 and 1,750, between 500 and 2,000, between 500 and 2,250, between 500 and 2,500, between 500 and 2,750, between 500 and 3,000, between 500 and 3,250, between 500 and 3,500, between 500 and 3,750, between 500 and 4,000, between 600 and 700, between 600 and 800, between 600 and 900, between 600 and 1,000, between 600 and 1,250, between 600 and 1,500, between 600 and 1,750, between 600 and 2,000, between 600 and 2,250, between 600 and 2,500, between 600 and 2,750, between 600 and 3,000, between 600 and 3,250, between 600 and 3,500, between 600 and 3,750, between 600 and 4,000, between 700 and 800, between 700 and 900, between 700 and 1,000, between 700 and 1,250, between 700 and 1,500, between 700 and 1,750, between 700 and 2,000, between 700 and 2,250, between 700 and 2,500, between 700 and 2,750, between 700 and 3,000, between 700 and 3,250, between 700 and 3,500, between 700 and 3,750, between 700 and 4,000, between 800 and 900, between 800 and 1,000, between 800 and 1,250, between 800 and 1,500, between 800 and 1,750, between 800 and 2,000, between 800 and 2,250, between 800 and 2,500, between 800 and 2,750, between 800 and 3,000, between 800 and 3,250, between 800 and 3,500, between 800 and 3,750, between 800 and 4,000, between 900 and 1,000, between 900 and 1,250, between 900 and 1,500, between 900 and 1,750, between 900 and 2,000, between 900 and 2,250, between 900 and 2,500, between 900 and 2,750, between 900 and 3,000, between 900 and 3,250, between 900 and 3,500, between 900 and 3,750, between 900 and 4,000, between 1,000 and 1,250, between 1,000 and 1,500, between 1,000 and 1,750, between 1,000 and 2,000, between 1,000 and 2,250, between 1,000 and 2,500, between 1,000 and 2,750, between 1,000 and 3,000, between 1,000 and 3,250, between 1,000 and 3,500, between 1,000 and 3,750, between 1,000 and 4,000, between 1,250 and 1,500, between 1,250 and 1,750, between 1,250 and 2,000, between 1,250 and 2,250, between 1,250 and 2,500, between 1,250 and 2,750, between 1,250 and 3,000, between 1,250 and 3,250, between 1,250 and 3,500, between 1,250 and 3,750, between 1,250 and 4,000, between 1,500 and 1,750, between 1,500 and 2,000, between 1,500 and 2,250, between 1,500 and 2,500, between 1,500 and 2,750, between 1,500 and 3,000, between 1,500 and 3,250, between 1,500 and 3,500, between 1,500 and 3,750, between 1,500 and 4,000, between 1,750 and 2,000, between 1,750 and 2,250, between 1,750 and 2,500, between 1,750 and 2,750, between 1,750 and 3,000, between 1,750 and 3,250, between 1,750 and 3,500, between 1,750 and 3,750, between 1,750 and 4,000, between 2,000 and 2,250, between 2,000 and 2,500, between 2,000 and 2,750, between 2,000 and 3,000, between 2,000 and 3,250, between 2,000 and 3,500, between 2,000 and 3,750, between 2,000 and 4,000, between 2,250 and 2,500, between 2,250 and 2,750, between 2,250 and 3,000, between 2,250 and 3,250, between 2,250 and 3,500, between 2,250 and 3,750, between 2,250 and 4,000, between 2,500 and 2,750, between 2,500 and 3,000, between 2,500 and 3,250, between 2,500 and 3,500, between 2,500 and 3,750, between 2,500 and 4,000, between 2,750 and 3,000, between 2,750 and 3,250, between 2,750 and 3,500, between 2,750 and 3,750, between 2,750 and 4,000, between 3,000 and 3,250, between 3,000 and 3,500, between 3,000 and 3,750, between 3,000 and 4,000, between 3,250 and 3,500, between 3,250 and 3,750, between 3,250 and 4,000, between 3,500 and 3,750, or between 3,750 and 4,000.

Individual components of compositions or nanocomposites will now be described in additional detail. The nanoparticle of a composition or nanocomposite described herein can have any size not inconsistent with the technical objectives of the present disclosure. For example, in some implementations, the average size of the nanoparticle in three dimensions is between 0.1 μm and 5 μm. In some embodiments, the average size of the nanoparticle in three dimensions is between 0.2 μm and 5 μm or between 0.2 μm and 2 μm.

Further, in some embodiments, a nanoparticle described herein has a surface charge or surface charge density suitable for making and/or using compositions or nanocomposites described herein. For example, in some instances, the exterior surface of the nanoparticle has an opposite charge compared to a solvent-facing charge density of the plurality of nanofibers. In some preferred embodiments, the exterior surface of the nanoparticle is negatively charged or has a negative zeta potential, and the nanofibers have a positive solvent-facing charge density or a positive zeta potential.

A nanoparticle described herein may also comprise or be formed from any material not inconsistent with the technical objectives of the present disclosure. In some implementations, the nanoparticle of a composition described herein is formed from a biocompatible and/or biodegradable material. In some instances, a nanoparticle comprises a lipid nanoparticle or a liposome. In some such cases, the lipid nanoparticle or liposome has a negative solvent-facing charge density.

In other embodiments, the nanoparticle of a composition described herein is formed from an inorganic material. For example, in some cases, the nanoparticle is formed from a ceramic material, a mixture or combination of ceramic materials, a bioglass, a metal, a mixture, combination, or alloy of metals, or a combination of two or more of the foregoing. In some embodiments, a nanoparticle described herein is formed from SiO₂, TiO₂, ZrO₂, CaO, MgO, Na₂O, K₂O, P₂O₅, hydroxyapatite (Ca₁₀(PO₄)₆(OH)₂), stainless steel, a cobalt-chromium alloy, titanium, a titanium alloy, a silicone, or a combination of two or more of the foregoing.

In still other cases, the nanoparticle of a composition or nanocomposite described herein is formed from an organic material. In some such instances, the nanoparticle is formed from a polymer, such as a polyvinylchloride (PVC), a polyethylene (PE), a polypropylene (PP), a polytetrafluoroethylene (PTFE), a polymethylmethacrylate (PMMA), a poly(trimethylene carbonate) (PTMC), a poly(lactic-co-glycolic acid) (PLGA), a poly(lactic acid) (PLA), a poly(glycolic acid) (PGA), a polysaccharide, or a combination or mixture of two or more of the foregoing.

Moreover, in some embodiments described herein, a nanoparticle is porous. Such a porous nanoparticle, in some cases, comprises pores permitting diffusion of a payload and/or solvent into and/or out of the interior of the nanoparticle. In this manner, a porous nanoparticle can release its payload into a biological compartment or into the cytosol of a cell over a desired time period, as described further hereinbelow.

A nanoparticle can be present in a composition or nanocomposite described herein in any amount not inconsistent with the technical objectives of the present disclosure. For example, in some cases, the nanoparticle component is present in the composition or nanocomposite in an amount of 10-90 wt. %, 10-80 wt. %, 10-70 wt. %, 10-60 wt. %, 10-50 wt. %, 20-90 wt. %, 20-80 wt. %, 20-70 wt. %, 20-60 wt. %, 20-50 wt. %, 20-40 wt. %, 30-90 wt. %, 30-80 wt. %, 30-70 wt. %, 30-60 wt. %, or 30-50 wt. %, based on the total weight of the composition. Moreover, in some such instances, the combined weight of the nanoparticle, nanofibers, and payload is at least 90 wt. %, at least 95 wt. %, or at least 99 wt. % of the overall composition (that is, in such instances there is no more than 10%, no more than 5%, or no more than 1% additional component present in the overall composition, other than the nanoparticle component, nanofibers component, and payload component, as well as any solvent encapsulated within the nanoparticle along with the active or functional components of the payload).

Turning now to nanofibers of a composition described herein, the nanofibers can have any size and shape not inconsistent with the technical objectives of the present disclosure. For example, in some cases, the average length of the nanofibers in the long dimension is between 20 nm and 50 nm. It is to be understood that the “length” or “average length” of the plurality or population of nanofibers is the spatial extent or size of the nanofibers in the “long” dimension, meaning the one dimension of the nanofibers (e.g., denoted as the “z” dimension) that is long relative to the other two dimensions (e.g., denoted as the “x” and “y” dimensions). Additionally, in some embodiments described herein, the average width of the nanofibers in one or two dimensions (e.g., in either the “x” dimension or in the “y” dimension, or in each of the “x” dimension and the “y” dimension) is less than 10 nm. In some instances, the average width of the nanofibers is 1-10 nm, 3-7 nm, or 3-5 nm. Moreover, nanofibers described herein can have any shape or cross-section not inconsistent with the technical objectives of the present disclosure. In some cases, for example, the nanofibers have a rectangular cross-section (in the two relatively short directions, as opposed to the one relatively long direction), and a specific width size recited herein is an average of both such short dimensions or corresponds to one of the two short dimensions (e.g., a width or height, as compared to the length).

Nanofibers of a composition described herein can be formed from any material not inconsistent with the objectives of the present disclosure. In some preferred embodiments, the nanofibers comprise polypeptide nanofibers. Moreover, such polypeptides can comprise particularly selected numbers and/or types of amino acid residues. For example, in some cases, the polypeptide nanofibers comprise 15 to 40 residues per peptide chain. In some instances, the polypeptide nanofibers comprise 20 to 40, 20 to 35, 21 to 40, 21 to 35, or 21 to 32 residues per peptide chain.

Additionally, in some implementations, the nanofibers of a composition or nanocomposite described herein comprise self-assembled polypeptide nanofibers. In some preferred embodiments, the nanofibers comprise multidomain peptides (MDPs), such as described in Yang et al., “Modular design and self-assembly of multidomain peptides towards cytocompatible supramolecular cell penetrating nanofibers,” RSC Adv., 2020, 10, 29469, the entirety of which is hereby incorporated by reference. Moreover, in some preferred embodiments, the nanofibers have a peptide sequence of Kₓ(QW)₆Ev, where x is an integer ranging from 8 to 15 and y is an integer ranging from 1 to 5 (SEQ ID NO: 4). In some such cases, x is an integer ranging from 8 to 10 and y is an integer ranging from 1 to 3. In some especially preferred embodiments, the nanofibers have a peptide sequence of K₁₀(QW)₆E₃ (SEQ ID NO: 1). As understood by a person of ordinary skill in the art, the letters K, Q, W, and E above refer, respectively, to the 1-letter denotations of lysine (K; corresponding to 3-letter abbreviation Lys), glutamine (Q, corresponding to 3-letter abbreviation Gln), tryptophan (W, corresponding to 3-letter abbreviation Trp), and glutamate/glutamic acid (E, corresponding to 3-letter abbreviation Glu), in accordance with standard amino acid nomenclature, including IUPAC-IUBMB nomenclature.

The nanofiber component can be present in a composition or nanocomposite described herein in any amount not inconsistent with the technical objectives of the present disclosure. In some embodiments, the nanofibers are present in the composition in an amount of 0.5 to 15 wt. %, based on the total weight of the composition. In some cases, the nanofibers are present in the composition in an amount of 1-10 wt. %, 3-15 wt. %, 3-12 wt. %, 3-10 wt. %, 4-15 wt. %, 4-12 wt. %, 4-10 wt. %, 4-8 wt. %, 5-15 wt. %, 5-12 wt. %, 5-10 wt. %, 5-8 wt. %, based on the total weight of the composition. In some such instances, the combined weight of the nanoparticle, nanofibers, and payload is at least 90 wt. %, at least 95 wt. %, or at least 99 wt. % of the overall composition.

Compositions or nanocomposites described herein also comprise a payload. It is to be understood that such a “payload” can comprise a chemical species, component, or agent (or combination of two or more such species, components, or agents) that exits the nanoparticle and is delivered to a biological compartment of a patient as described herein, or that remains encapsulated within the nanoparticle but provides functionality (e.g., fluorescence) to the overall composite or nanoparticle. In some embodiments, the payload is physically entrapped within the interior of the nanoparticle. Moreover, in some cases, the payload is operable to diffuse out of the interior of the nanoparticle when the composition is disposed in an aqueous or biological environment, as described further herein.

Any payload not inconsistent with the technical objectives of the present disclosure may be used in a composition or nanocomposite described herein. In some embodiments, the payload comprises an imaging agent, a therapeutic agent, a theranostic agent, or a combination of two or more of the foregoing. In some instances, for example, the payload comprises an imaging agent, and the imaging agent is luminescent (e.g., fluorescent or phosphorescent). Any luminescent imaging agent not inconsistent with the technical objectives of the present disclosure may be used. In some embodiments, an imaging agent comprises a molecular dye having a luminescence emission in the visible or infrared (IR) region of the electromagnetic spectrum (e.g., having a peak emission wavelength between 400 nm and 800 nm, or between 840 nm and 1500 nm), such as an indocyanine dye (e.g., indocyanine green), a rhodamine dye (such as rhodamine B), a coumarin dye, fluorescein, or methylene blue. In some instances, an imaging agent comprises a luminescent biomolecule, such as green fluorescent protein (GFP). Additionally, in some cases, an imaging agent comprises a luminescent quantum dot or other luminescent nanoparticle, such as a quantum dot or other luminescent nanoparticle having an average size in three dimensions of less than 15 nm or less than 10 nm. An imaging agent may also comprise a contrast agent (e.g., an MRI contrast agent), such as a lanthanide compound or complex. Other imaging agents may also be used, and the imaging agent is not particularly limited.

A therapeutic agent used in a composition described herein, in some implementations, comprises a small molecule drug or other molecular drug (e.g., vancomycin), which may be hydrophobic or hydrophilic or amphiphilic. In some instances, a therapeutic agent comprises a nucleic acid, such as a small interfering ribonucleic acid (siRNA). Other therapeutic agents may also be used, and the therapeutic agent is not particularly limited.

Similarly, a variety of theranostic agents (agents that can provide both diagnosis, such as by luminescence or magnetic resonance imaging (MRI), and also treatment, such as by hyperthermia, chemotherapy, or gene therapy) may be used in a composition or nanocomposite described here. The theranostic agent is not particularly limited.

In other cases, the payload comprises nucleic acids, proteins, peptides, chemotherapeutics, vaccine components, antibiotics, or a combination of two or more of the foregoing. In some instances, for example, nucleic acids comprise DNA, cDNA, RNA, mRNA, miRNA, iRNA, siRNA, ribozymes, plasmids, aptamers, anti-sense nucleic acid, peptide-nucleic acids, or oligonucleotides, antisense oligonucleotides, DNAzymes, antagomirs (anti-miRs), miRNA mimics, supermirs, or aptamers.

The payload component of a composition or nanocomposite described herein can be present in any amount not inconsistent with the technical objectives of the present disclosure. In some embodiments, for example, the payload is present in the composition in an amount of 1-80 wt. %, based on the total weight of the composition. In some cases, the payload is present in an amount of 1-70 wt. %, 5-80 wt. %, 5-70 wt. %, 5-50 wt. %, 5-40 wt. %, 5-30 wt. %, 5-25 wt. %, 5-20 wt. %, 5-15 wt. %, 10-80 wt. %, 10-70 wt. %, 10-60 wt. %, 10-50 wt. %, 20-80 wt. %, 20-70 wt. %, 20-60 wt. %, 20-50 wt. %, 20-40 wt. %, 30-80 wt. %, 30-70 wt. %, 40-80 wt. %, 40-70 wt. %, or 40-60 wt. %, based on the total weight of the composition. Moreover, in some instances, the payload comprises a hydrophilic species such as a hydrophilic drug (e.g., vancomycin), and the hydrophilic species in present in an amount of 2-20 wt. %, 3-18 wt. %, or 5-15 wt. %, based on the total weight of the composition. In other embodiments, the payload comprises a hydrophobic species such as a hydrophobic drug, and the hydrophobic species in present in an amount of greater than 30 wt. %, greater than 40 wt. %, or greater than 50 wt. %, based on the total weight of the composition. In some implementations, a hydrophobic payload is present in an amount of 30-80 wt. % or 40-70 wt. %, based on the total weight of the composition. Moreover, in some such embodiments as described in this paragraph, the combined weight of the nanoparticle, nanofibers, and payload is at least 90 wt. %, at least 95 wt. %, or at least 99 wt. % of the overall composition (where solvent included with the payload may be considered to be part of the total payload amount).

In another aspect, methods of treating and/or diagnosing a condition or disease in a patient in need thereof are described herein. In some embodiments, such a method comprises disposing a composition or nanocomposite described herein within a biological compartment of the patient. Any composition or nanocomposite described herein may be used. Additionally, the biological compartment can be any suitable biological compartment of the patient, such as an internal organ of the patient. A composition described herein may also be disposed in or delivered to the bloodstream of the patient or in or to a blood vessel of the patient. Disposing or delivering the composition or nanocomposite can be carried out in any manner not inconsistent with the objectives of the present disclosure. In some cases, for example, the composition or nanocomposite or injected into the biological compartment. In some preferred embodiments, the composition is disposed within the biological compartment of the patient by inhalation or nebulization, and injection is avoided.

In some cases, a method described herein further comprises penetrating a membrane of a cell or population of cells within the biological compartment with the plurality of nanofibers of the composition. That is, the nanofibers enable or permit uptake of the overall nanocomposite with a cell or population of cells (e.g., within the lungs of a patient). Additionally, in some implementations, a method described herein further comprises releasing at least a portion of the payload of the composition within a cytosol of the cell or population of cells after penetrating the membrane of the cell or population of cells. Further, in some cases, a method described herein also comprises biologically degrading the nanoparticle and/or the plurality of nanofibers of the composition after penetrating the membrane of the cell or population of cells. Moreover, in some cases, the payload of a composition used in a method described herein comprises an imaging agent or a theranostic agent, and the method further comprises imaging the cell or population of cells with the imaging agent or theranostic agent, which may occur before, during, or after release of a payload or penetration within a cell or population of cells.

In some exemplary embodiments of a method, as described further below, the condition or disease comprises a respiratory condition or disease, and the biological compartment is a pulmonary site. Additionally, in some such cases, the respiratory condition or disease comprises a degenerative or genetic disease. In some embodiments, the respiratory condition or disease comprises idiopathic lung fibrosis, a chronic obstructive pulmonary disease (COPD), or a lung cancer. Moreover, in some implementations, the respiratory condition or disease is caused by a pathogen or product of a pathogen, and the payload comprises a therapeutic agent effective for the treatment of the condition or disease caused by the pathogen or product of the pathogen. For example, in some instances, the pathogen or product of the pathogen comprises one or more of Methicillin-Resistant Staphylococcus Aureus (MRSA), Alpha-toxin (Hla), Staphylococcal protein A (Spa), and SARS-CoV-2. In other cases, the respiratory condition or disease comprises Mycobacterium tuberculosis and/or Streptococcus pneumonia, and the pathogen or product of the pathogen comprises mycobacterium and/or streptococcus bacterium.

Some features and characteristics of the various embodiments according to the present disclosure are described in further detail in the specific Examples below. These Examples are not meant to limit embodiments solely to such Examples herein, but rather to illustrate some possible implementations

### EXAMPLE 1

Fiber-Forming Supramolecular Cell Penetrating Peptide-Coated onto PLGA Nanoparticles for Enhanced Pulmonary Drug Delivery

### A. Introduction

Nanoparticles (NPs) with high surface area-to-volume ratio can be employed to deliver drugs and other therapeutics. Drug encapsulating NPs can increase drug bioavailability and drug release in targeted tissues. This can be highly beneficial to reduce dosing frequency, improving patient compliance. NPs include polymer-based particles, dendrimers, liposomes, metal-based particles, and inorganic particles like silica, among others. Although NPs are capable of entering cells through different endocytosis mechanisms, there can be an issue of tuning the number of NPs needed to exert a therapeutic effect. Engineering strategies improving the uptake of nanoparticles can have a profound effect on drug delivery towards diseased cells including infected, senescent, cancerous, and other abnormalities where an altered uptake ability or even a reduced uptake ability is seen.

Cell penetrating peptides (CPPs) have an ability to cross the cell membrane for intracellular drug delivery. NPs modified with CPPs can increase internalization in cells for various applications including targeting and imaging of cancer. NPs can be modified with CPPs by two major strategies, electrostatic interactions and covalent crosslinking, such as click chemistry. Cationic CPPs, such as the transactivator protein (Tat) of human deficiency virus (HIV), can be used in modifying NPs. Arginine-rich peptides can also be used to modify nanoparticles; for instance, it is possible to directly cross-link the peptide thiol group to the surface of gold nanoparticles for cancer therapy. It is also possible to decorate NPs with tumor-homing and penetrating peptide-F3 for theragnostic purposes. The F3-peptide coating on NPs can enhance cell association and preferential targeting to the tumor site, providing a multimodal therapy for cancer treatment. Dual peptides of CPP Tat and antagonist G peptide can be conjugated onto polymer PLGA NPs with the use of EDC-NHS click chemistry. Overall, peptide-modified NPs can improve targeting and therapeutic efficacy of NPs.

However, most natural and synthetic CPPs are active in the monomeric form, which leads to lower binding affinity toward NPs and rapid enzymatic degradation. High concentrations of CPPs are needed to either covalently or noncovalently attach onto NPs, which may cause high cytotoxicity. Peptide self-assembly can provide an effective method to generate supramolecular nanomaterials with improved stability, dynamic nanostructure, and biological activity.

In this example, a novel nanocomposite (NC) of fiber-forming supramolecular cell penetrating peptide nanofibers (NFs) that are coated onto polylactic-glycolic acid (PLGA) nanoparticles was used to enhance pulmonary drug delivery (FIG. 1). NFs bind to FDA-approved biodegradable PLGA and NPs electrostatically to form NCs and enhance the uptake ability of NPs with payloads for intracellular drug delivery. These nanocomposites show a 3-fold higher intracellular delivery of nanoparticles in various cell lines, including primary lung epithelial cells, macrophages, and a 10-fold increase in endothelial cells compared to naked PLGA nanoparticles or a 2-fold increase compared to nanoparticles modified with traditional monomeric cell-penetrating peptides (CPPs). Cell uptake studies showed that nanocomposites may enter cells through mixed macropinocytosis and passive energy independent mechanisms, which is followed by endosomal escape within 24 hours. Nanocomposites also had potent mucus permeation. Additionally, freeze-drying and nebulizing formulated nanocomposite powder did not affect their physiochemical and biological activity, which further highlights the translative use as a stable drug carrier for pulmonary drug delivery. Nanocomposites based on peptide nanofibers and PLGA nanoparticles, as described herein, can be custom designed to encapsulate and deliver a wide range of therapeutics including nucleic acids, proteins, and small molecule drugs when employed in inhalable systems to treat various pulmonary diseases.

### B. Experimental Section

### Synthesis of Nanocomposites

A double emulsion method as described by Messerschmidt et al. was employed for the synthesis of PLGA NP. First, 100 mg of PLGA polymer (copolymer ratio 50:50, molecular weight 15 kDa-25 kDa) was dissolved in dichloromethane at 100 mg/mL. 1% (w/w) Rhodamine B (Rho B) was prepared as a water phase, which was later added dropwise into the oil-phase of the PLGA solution. This primary solution was sonicated to form the primary emulsion. The primary emulsion was emulsified into 5% (w/v) poly(vinyl) alcohol (PVA, 13 kDa) solution via sonication at 35 watts for 4 minutes (30 seconds off every 1 minute). Rho B-loaded PLGA nanoparticles were collected by centrifugation at 15,000 rpm for 15 minutes and then lyophilized until completely dry.

Nanofibers were synthesized as previously described [29]. Briefly, a standard Fmoc-solid phase peptide synthesis method was employed, and the synthesis was carried out on a Prelude peptide synthesizer. The peptide was terminated with either an acetyl group or FITC. The acetylated peptide is denoted as non-labeled peptide, and the FITC-terminated peptide is denoted as labeled peptide for the following procedures. All peptides were purified by high performance liquid chromatography (HPLC) followed by lyophilization. The molecular weight of each peptide was confirmed by MALDI-TOF mass spectrometry using α-cyano-4-hydroxycinnamic acid as the matrix (Acetylated K₁₀(QW)₆E₃: expected [M+H]⁺: 3612.9, observed [M+H]⁺: 3611.2; FITC-terminated K₁₀(QW)₆E₃: expected [M+H]⁺: 3928.9, observed [M+H]⁺: 3629.23).

Non-labeled peptides were dissolved in tris(hydroxymethyl)aminomethane) (Tris) buffer (pH 7.4, 20 mM) buffer at 1 mM concentration and incubated for a period of 12 hours for self-assembly into nanofibers. Nanofibers containing labeled peptides were prepared by mixing non-labeled peptide with FITC-labeled peptide with a molar ratio of 90:10 in a mixed solvent of water and acetonitrile (1:1 by volume). The mixture was lyophilized, rehydrated in Tris buffer (pH 7.4, 20 mM) to reach a final concentration of 1 mM, and left at 4° C. for 12 hours.

After lyophilization of nanoparticles, 2 mg of Rho B-NPs were dissolved in Tris buffer, and 0.5 mg of nanofiber in suspension was added to the nanoparticle suspension. The mixture was left to react electrostatically by rotating the solution for an hour at room temperature. Later, the sample was centrifuged at 15,000 rpm for 7 minutes to remove free nanofibers and collect the nanocomposites that contained nanofiber-coated Rho B-loaded nanoparticles.

Plain PLGA NPs used for FTIR studies were synthesized by a single emulsion method in which the PLGA polymer was dissolved in chloroform followed by dropwise addition into 5% (w/v) PVA. The mixture was emulsified via sonication at 35 watts for 4 minutes (30 seconds off every 1 minute). Later, the PLGA NPs were collected via centrifugation and lyophilized until dry.

### Characterization of Nanocomposites

### DLS Measurements

A ZETAPALS90 dynamic light scattering (DLS) detector (Brookhaven Instrument, Holtsville, NY) was used to determine the size, charge, and polydispersity of the nanocomposites. For DLS measurements, 50 μL of 1 mg/mL nanocomposite suspension was mixed with 3 mL of DI water in a transparent cuvette and placed in the instrument to measure size, while a DLS probe was used to measure the zeta potential of the nanocomposites.

### Fluorescent Microscopy

Fluorescein-terminated peptides were synthesized as previously described by Yang et al. FITC-tagged peptides were mixed with Rho B PLGA NPs. Green color-tagged nanofibers were incubated with nanoparticles loaded with rhodamine B (red color). The nanocomposites formed were washed 3 times to remove any unbound nanofibers. Another set of nanoparticles were similarly washed and imaged without any nanofibers. A fluorescent microscope with channels for FITC (for the nanofibers) and Texas Red (for Rho B NPs) was used to image the nanofiber coating on the nanoparticles.

### Cryo-Electron Microscopy

Cryo-EM grids were prepared using a Vitrobot Mark IV plunge-freezer (ThermoFisher Scientific). Three μL of the sample were applied to Lacey carbon grids (300-mesh; Ted Pella, Inc.) that were glow discharged at 30 mA for 80 s. The grids were blotted at 95% relative humidity for 4 s prior to plunge freezing. The sample grids were imaged on a Talos Arctica 200 kV transmission electron microscope (ThermoFisher Scientific) equipped with a Gatan K3 camera (Gatan, Inc.). The nominal magnification is at 45,000x, which corresponds to a pixel size of 0.88 Å.

### FTIR of Nanocomposites

Freeze-dried material including PLGA polymers, plain PLGA nanoparticles, nanocomposites, and nanofibers were analyzed using Fourier-Transform infrared spectroscopy (FTIR). Briefly, FTIR spectra of the varied materials were recorded in transmission mode using a Nicolet 6700 in the range of 400 to 4000 cm⁻¹.

### Binding Kinetics of Nanofibers to Nanoparticles

A thermophoresis technique was used to detect the binding of nanofibers (ligand) to the nanoparticles. Fₙₒᵣₘ represents the change in thermophoresis, which is expressed as change in thermophoresis when non-fluorescent ligand titration is introduced to fluorescent nanoparticles. Here, nanofiber titrations were made starting from 2 mg/mL of nanofibers up to 10 dilutions with the nanoparticle concentration kept at 2 mg/mL for all the titrations. A small capillary tube was used to load approximately 4 μL of the various nanofiber-nanoparticle combinations and placed in the loading tray of a thermophoresis instrument Monolith NT.115 (NanoTemper Technologies, Inc., San Francisco, CA). To determine the position of the capillaries, a fluorescence scan was performed. Subsequently, thermophoresis measurements were performed to determine the binding kinetics of nanofibers to nanoparticles. Fₙₒᵣₘ was calculated by the machine along with various other parameters, including the binding constant.

### Cytocompatibility of Nanocomposites

Primary lung epithelial cells, RAW macrophages, and HUVECs were used to assess toxicity of the nanocomposites. Nanocomposites were prepared as described in for AT1 cells, in which 20,000 cells/well of primary alveolar type I epithelial cells (AT1) were seeded in 48-well plates. After the overnight culture, various groups of particles including blank PLGA NPs, nanofibers only, and nanocomposites (nanofiber coated-PLGA nanoparticles) were given to the cells in triplicate at various concentrations ranging from 0.0625-1 mg/mL. The nanofiber concentration chosen was equivalent to the peptide amount conjugated onto the nanoparticles. After 72 hours, the cells were washed 3 times with PBS, and MTS reagent was applied to the cells to assess the cell viability following the company's instructions.

### Cellular Uptake of Nanocomposites

Cellular uptake studies were performed as described previously by Iyer et al. Nanocomposites made from rhodamine B PLGA NPs and nanofibers were used as fluorescently labeled nanocomposites for cell uptake studies. Cellular uptake of nanocomposites was determined by measuring internalized fluorescent nanocomposites. Various cell lines representative of the lower respiratory tract, including AT1 and RAW macrophages, were used to assess the nanocomposite cell internalization ability compared with plain/blank nanoparticles. AT1 cells (15,000 cells/well) and RAW cells (20,000 cells/well) were seeded onto a 48-well plate and grown overnight at 37° C. After overnight attachment, various nanoparticles and nanocomposites at different concentrations (0, 50, 100, and 250 μg/mL) in media were applied to the cells for 90 minutes. After 90 minutes, the cells were washed 3 times with PBS and lysed using 2% Triton X-100. The fluorescence intensities of internalized NPs or NCs were measured at an excitation wavelength of 546 nm and emission wavelength of 585 nm for the rhodamine B fluorescence loaded into the NPs and NCs. The cell lysate was also used to determine the protein content using bicinchonic acid assays (BCA) per the manufacturer's instructions (Pierce™ BCA Protein Assay, ThermoScientific).

For visualizing the cellular uptake before the cell lysis, the cells were stained for the nucleus with NucBlue (ThermoScientific) for 20 minutes. After staining, the cells were imaged using an ECHO fluorescent microscope (ECHO, San Francisco, CA) using the DAPI channel for the nucleus and the Texas Red channel for NPs or NCs.

Time-dependent uptake of nanocomposites in comparison with nanoparticles was observed for lung epithelial cells. Briefly, AT1 cells were seeded at confluency and allowed to attach overnight. The next day, cells were treated with 0.5 mg/mL of nanocomposites and nanoparticles for uptake. At timepoints of 30 minutes, 90 minutes and 4 hours, the cells were washed 3 times with PBS and lysed with 2% Triton X-100. The cell lysates were read for fluorescence using a plate reader. Later, the total cell protein content was measured using a BCA assay.

AT1 cells were seeded at confluency onto a glass slide to perform confocal studies for nanocomposite uptake. After overnight attachment, nanocomposites or nanoparticles at a concentration of 0.5 mg/mL were incubated with the cells for uptake. Cells were washed with PBS 3 times after 4 hours of uptake, and the cell nucleus was stained with NucBlue. Cells on the glass slide were mounted with a cover slip to visualize the internalization of nanocomposites using a Nikon A1R confocal microscope.

AT1 cells were seeded on a confocal dish to perform a lysosome escape study. After overnight attachment, nanocomposites were incubated with the cells at a concentration of 0.5 mg/mL. For all incubation times, the medium was changed at 2 hours to maintain consistent cell uptake quality. After 2, 8, and 24 hours incubation, cells were washed with PBS 3 times. The lysosome was stained with LysoTracker™ Blue DND-22 (Invitrogen) for 30 minutes and followed by washing with PBS 3 times. The internalization of nanocomposites was visualized using a Nikon A1R confocal microscope.

### Nanocomposite Cellular Uptake Mechanism Study

To determine the NC/NP uptake mechanism used by cells, an endocytosis-inhibition examination was performed. Alveolar Type 1 cells were seeded at confluency in 48-well plates and attached overnight at 37° C. in an incubator with 5% CO₂. Rhodamine B-loaded nanocomposites were prepared using a similar procedure done for other studies. After 24 hours of seeding, the AT1 cell culture medium was replaced by fresh 1% serum media containing 5 μM of Amiloride, 5 μM of methyl-β-cyclodextran, 5 μg/mL of Filipin III, 5 μM of Cytochalasin-D, 5 μM of Imipramine, 80 μM Dynasore, or 5 mM of Deoxy-glucose (Sigma Aldrich & Cayman Chemical). After 2 hours, fresh complete media with Rho B-labeled nanocomposites at a concentration of 0.5 mg/mL was added. After 12 hours, the cells were washed with PBS, and the nuclei were stained with NucBlue (ThermoFischer). Images were acquired using a fluorescent microscope and processed for NP uptake in random areas using 10 images from each inhibitor group with the ImageJ software.

In a similar fashion, to check if the nanocomposites undergo an energy-dependent uptake, temperature block was studied in AT1 cells by preincubating cells at 4° C. for 30 minutes, followed by treatment with fluorescent nanocomposites for 90 minutes at 4° C. Later, fluorescent images were acquired, and the cell lysates were analyzed for quantitative nanocomposite uptake.

### Mucus Permeation Study

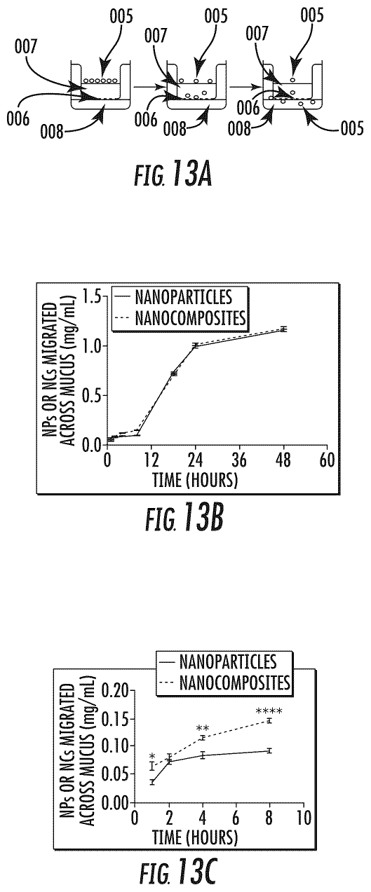

A mucus permeation study was performed to study the effects of the nanofiber coating on the permeation of the nanoparticles to mimic the in vivo environment. FIG. 13A shows the setup used. NCs/NPs loaded with rhodamine B dye (005) were used for the study. A 12-well transwell plate with a polycarbonate membrane with a pore size of 0.4 μm (006) was used for the study. Mucus was simulated based on a method in previous literature [57]. Porcine mucin protein was mixed with salts, DNA, and DPPC to form simulated mucus (007). 100 μL of simulated mucus was placed on the transwell membrane, and 500 μL of PBS was placed in the lower chamber (008). 25 μL of 10 mg/mL of NCs/NPs (005) were placed on top of the simulated mucus to allow for permeation from the transwell to the lower chamber. PBS from the lower chamber was collected at various timepoints to measure the level of fluorescent NCs/NPs that permeated through the mucus. Fresh PBS was replaced at different timepoints.

### In Vitro Nebulization

To assess the ability of cells to uptake aerosolized NCs, a nebulizer was used to deliver the nanocomposites and nanoparticles. AT1 lung epithelial cells were seeded at 0.4 million cells/well in a 12-well plate and grown overnight. 1 mg/mL of both NCs and NPs in PBS were aerosolized using a lab module nebulizer from Aeroneb® (Kent Scientific, Torrington, CT). Aeroneb generated 2.5-4 μm droplets of particulate suspension. After the nebulization of droplets, cells were incubated at 37° C. for 90 minutes. After incubation, the cells were washed with PBS and stained for the nucleus with NucBlue (ThermoFisher). Fluorescent images for DAPI (nucleus) and Texas Red (NCs/NPs) staining were taken of the cells for uptake of nanocomposites and nanoparticles using a fluorescent microscope (ECHO, San Francisco, CA). Later, the cells were lysed using 2% Triton X-100, and the cell lysate was read using a spectrophotometer at an excitation wavelength of 546 nm and an emission wavelength of 585 nm. The cell protein amount measured by a protein assay was used to normalize the fluorescent readings from the cells. The percentage of total delivered (100%=NP/NC delivered to cells) and the weight number of NCs/NPs delivered to the cells were calculated based on the n cell protein normalized fluorescence readings.

### Effects of Nanocomposite Freeze-Drying

A cell uptake study was performed to assess the ability of nanocomposites to retain an enhanced uptake ability after freeze-drying. Nanoparticles along with nanocomposites loaded with rhodamine B dye were freeze-dried until dry. Later, NPs and NCs from before and after the freeze-drying were prepared using Tris buffer and later washed and mixed with complete media. AT1 cells in 48-well plates grown to confluency were given various groups of NCs and NPs from before and after freeze-drying samples at a concentration of 0.5 mg/mL. A cell uptake study was performed similar to the previous procedure.

Nanofiber-Coated NPs Vs. HIV Tat Peptide-Coated NPs

HIV Tat peptide is a common cell penetrating peptide. This study compared nanofiber coating and HIV Tat peptide coating for enhanced cell uptake ability. HIV Tat peptide (Sigma Aldrich, St. Louis, USA) coating of PLGA NPs was done similarly to the nanofiber coating. Briefly, 0.5 mg of either HIV Tat peptide or nanofiber was mixed with 2 mg of nanoparticles by rotation at room temperature for an hour. Later, the nanocomposites with the HIV Tat or nanofiber coating were collected and mixed with complete cell culture media at 0.5 mg/mL. AT1 cells grown overnight at confluency were given the nanocomposite groups and allowed to uptake for 90 minutes. A cell uptake study was performed similar to the previous procedure.

GraphPad Prism 8 (GraphPad Software Inc., San Diego, USA) was used to perform statistical analysis. One-way ANOVA with Sidak's multiple comparison, Dunnett multiple comparisons, and Tukey's multiple comparison tests were done for different data analyses as appropriate for data sample. Triplicate samples were used for all the studies if not specified.

### C. Results and Discussion

### Synthesis and Characterization of Nanocomposites

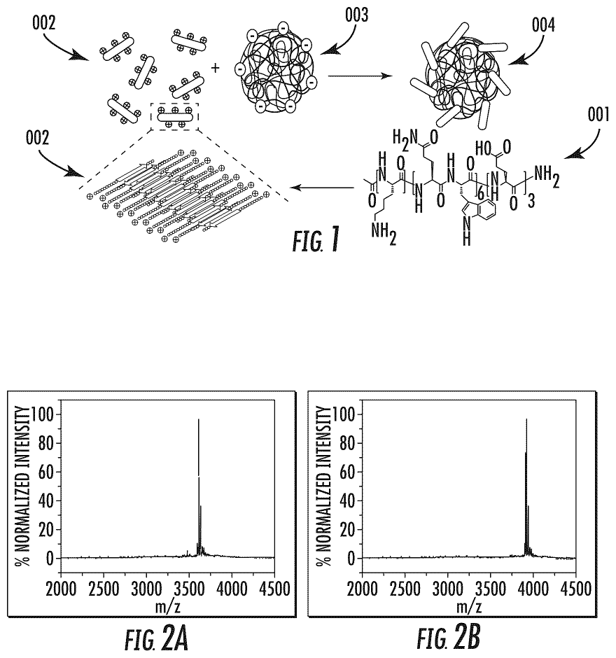

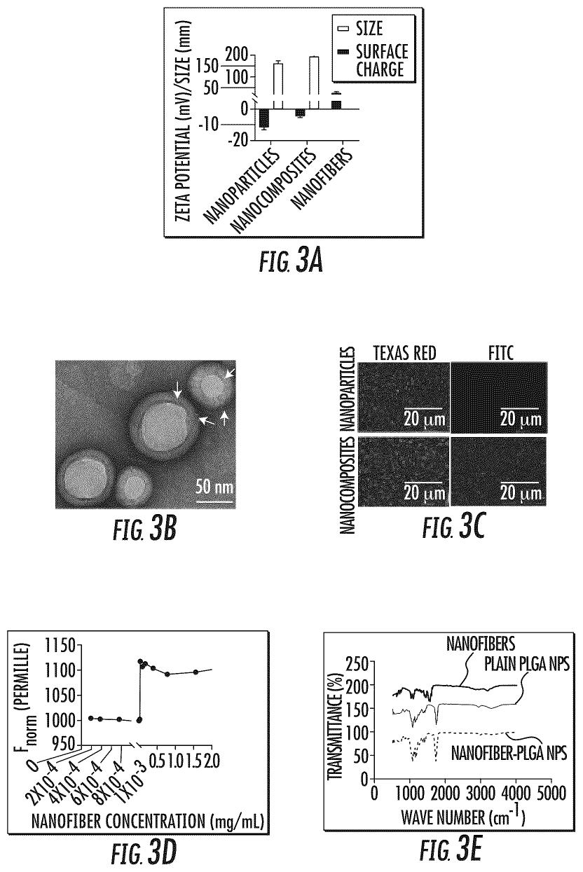

In this example, the synthesis and formulation of NCs involve complex formation of oppositely charged PLGA NPs and peptide NFs. As shown in FIG. 1, peptides (001) with a primary sequence of K₁₀(QW)₆E₃ (SEQ ID NO: 1) in which the subscript refers to the number of repeating units of each domain self-assemble to form nanofibers with a β-sheet structure (002). The nanofibers (002) have a positive charge and bind to the PLGA NPs (003) to form the nanofiber-nanoparticle nanocomposite (004). All peptides were synthesized on the solid phase and characterized by matrix assisted laser desorption/ionization-time of flight (MALDI-TOF) (FIG. 2A-B). The peptides are packed into a sandwich-like β-sheet structure with a net positively charged domain at the fiber-solvent interface. These NFs undergo charge complexation with biodegradable PLGA NPs to provide a novel pulmonary drug delivery system. PLGA NPs have high surface areas and can deliver high concentrations of drugs with a prolonged release in the lungs while avoiding systemic overdose from circulation through intravenous delivery. The PLGA NPs used in this Example have a size of ˜200 nm (FIG. 3A), which is suitable for lower respiratory tract delivery and avoiding exhalation and upper respiratory tract accumulation. For synthesis and in vitro testing of the NCs, PLGA nanoparticles ranging from 150-200 nm were employed. Negatively-stained transmission electron microscopy (TEM) images show the coating of NFs on the surface of the PLGA NPs in the TEM images (FIG. 3B). Cryo-TEM further confirmed the structure, although with a lower contrast (FIG. 4). This is notable evidence showing the stability of NFs upon physical interactions with PLGA NPs.

The interaction between NFs and NPs was further confirmed by the increase in size and reduction in the surface charge of NCs (FIG. 3A). The presence of peptide NFs on the PLGA NPs was also confirmed using FITC-labeled NFs (FIG. 3C). The results show FITC-labeled NFs co-localized with rhodamine B-labeled PLGA NPs (FIG. 3C).

Furthermore, to confirm the formation of NCs in which NFs are physically attached on the NPs, NPs were freeze-dried, and Fourier Transfer Infrared (FTIR) spectroscopy was performed to detect the presence of functional groups presented on the peptide NFs. An FTIR spectrum of NCs showed a distinct peak of amide I groups at 1640 cm⁻¹ along with (C—H) bending from the tryptophan rings seen at 750 cm⁻¹. These peaks are absent in PLGA NPs without NF coating (FIG. 3E). Characteristic IR absorption of PLGA was observed for the —CH aliphatic bond stretching at 2850-2950 cm⁻¹ and —C—O carbonyl stretching (FIG. 3E).

A microscale thermophoresis technique was used to investigate the interaction between NFs and PLGA NPs [39]. Here, thermophoretic kinetics of rhodamine B-labeled NPs in the presence of different concentrations of NFs were followed. As sample concentrations of nanofibers used increased, the movement of rhodamine B-labeled PLGA NPs was reduced in the presence of the infrared laser pertaining to the binding of NFs onto PLGA NPs, whereas at lower concentrations of NFs or for the NP only group, faster movement of NPs was shown with lower Fₙₒᵣₘ values (FIG. 3D). Lower Fₙₒᵣₘ values are seen until a concentration of 7.81×10⁻⁶ M, and then there is a jump showing higher Form from the interaction between NFs and NPs. A slight reduction of Fₙₒᵣₘ values was observed because of intrinsic tryptophan fluorescence from increased NF concentrations (Table 1). Any change in the initial fluorescence and background buffer fluorescence is verified by initial capillary scans. No fluorescence from buffer or parity in the initial fluorescence from test samples was observed (FIG. 5). Not intending to be bound by theory, it is believed the reduced movement of NPs in the presence of infrared laser is due to the bound NFs from electrostatic interactions with PLGA NPs.

TABLE 1
Data values for microscale thermophoresis.

| Concentration | |
|---|---|
| of nanofiber | Fₙₒᵣₘ |
| (μM) | (permille) |
| 0.0005 | 1113.397 |
| 0.00025 | 1096.287 |
| 0.000125 | 1091.279 |
| 6.25 × 10⁻⁵ | 1104.144 |
| 3.13 × 10⁻⁵ | 1112.34 |
| 1.56 × 10⁻⁵ | 1109.172 |
| 7.81 × 10⁻⁶ | 1116.518 |
| 3.91 × 10⁻⁶ | 1003.131 |
| 1.95 × 10⁻⁶ | 1000.085 |
| 9.77 × 10⁻⁷ | 999.0151 |
| 0 | 1001.368 |

### In Vitro Evaluation of NCs Uptake in Lung Epithelial Cells

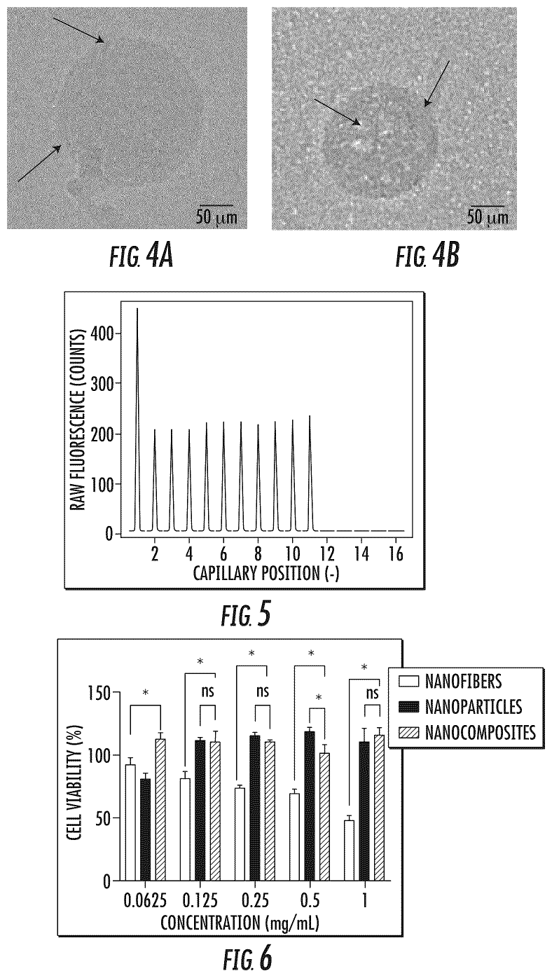

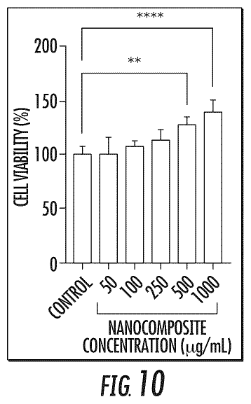

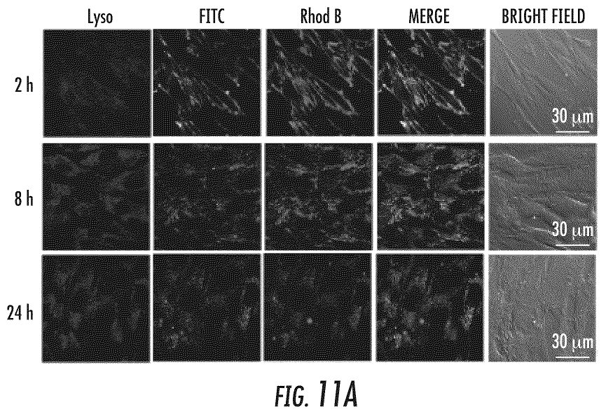

Lung epithelial cells were chosen as a model because of their significance in maintaining a barrier to circulation, which is often disrupted by various virulence factors such as Methicillin Resistant Staphylococcus Aureus (MRSA), Alpha-toxin (Hla), and Staphylococcal protein A (Spa) as well as viral pathogens such as SARS-CoV-2. Current lung disease treatment strategies involving systemic administration of drugs have low patient compliance and are associated with side effects. Hence, there is a need to develop effective drug delivery systems with the ability to overcome the various tissue/cell barriers for enhanced cell delivery efficacy. CPPs have been proven effective for intracellular drug delivery. However, at higher concentrations, they suffer from severe toxicity because of their membrane perturbations. Herein, the cytocompatibility of the new NCs were compared with NFs and NPs alone. NCs with a conjugation efficiency of ˜30% upon mixing of NFs and NPs (1:4 by mass or charge) were used for all the studies. Compared with plain NPs, the NCs did not show any significant change in cytocompatibility up to 1 mg/mL in primary lung AT1 epithelial cells compared to the untreated control, whereas NFs showed significant toxicity with concentrations ranging from 0.25-1 mg/mL (FIG. 6). NCs showed excellent cytocompatibility up to 1 mg/mL with RAW macrophages and HUVECs (FIG. 10). Based on the toxicity profile of NFs, NPs, and NCs, and not intending to be bound by theory, it is believed that the NCs interact with cells in a different mode compared to free NFs, which typically involve membrane disruption and permeation as the initial step toward cell-materials interactions [45]. Again not, intending to be bound by theory, the improved cytocompatibility is believed to be largely due to the reduction of the overall charge upon physical complexation of the positively charged NFs and negative charged NPs (FIG. 3A). Combining positively charged membrane-permeating NFs with cytocompatible NPs can reduce the membrane disruptive activity of NFs and alleviate their toxicity towards cells, in particular lung epithelial cells used in the current example for pulmonary drug delivery. It is also noteworthy to mention that although the overall charge of NCs is still negative, the cell uptake efficacy was dramatically enhanced, which will be discussed below.

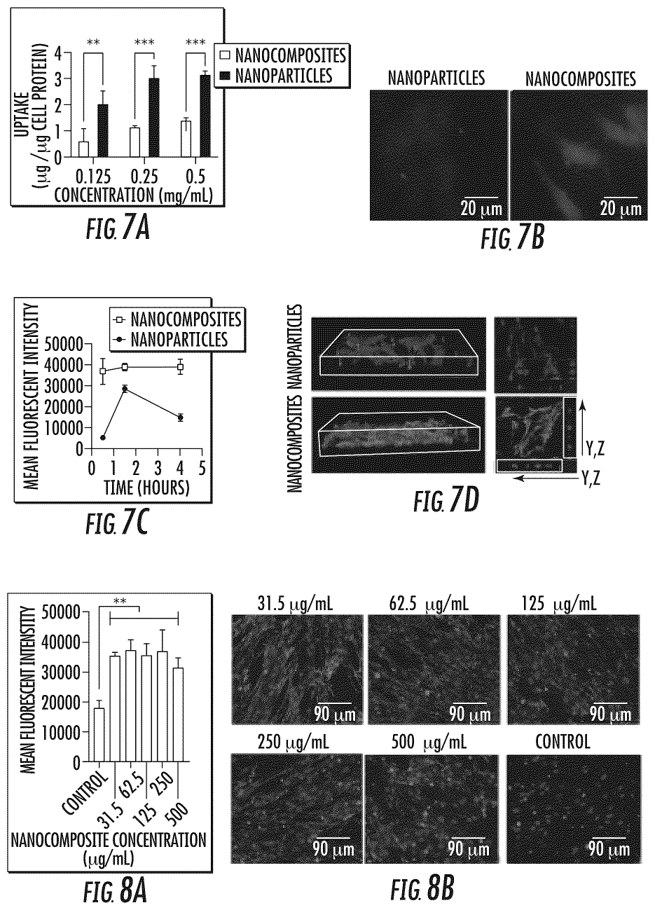

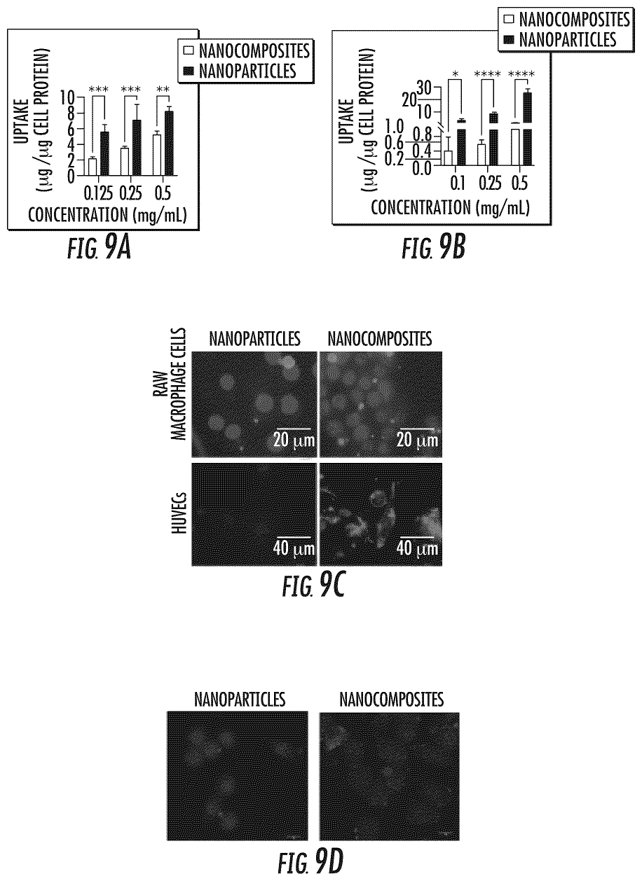

The ability of NCs to improve cellular uptake was studied in primary lung epithelial cells, RAW macrophages, and HUVECs. These commonly found cell lines were used to assess the enhanced drug delivery abilities of NCs towards pulmonary drug delivery. Both AT1 and RAW macrophages showed a 3-fold and 2-fold increased uptake of NCs (labeled with rhodamine B dye) compared to NPs (also labeled with rhodamine B dye), respectively (FIG. 7A, FIG. 8A, and FIG. 8B). RAW macrophages, because of their intrinsic phagocytic activity, showed a higher uptake of NCs compared to lung epithelial cells (FIG. 9A). Nanocomposites clearly had increased uptake in epithelial, macrophage and endothelial cells, which are the major cell types encountered when treating for pulmonary pathologies (FIG. 7B and FIG. 9A-D). A dose-dependent study with various concentrations of NCs (30-500 μg) using epithelial cells showed that even at a ratio of 1:128, NFs and NPs still can improve the intracellular delivery of NCs indicating the superior ability of NFs to interact with the cell membrane to internalize nanoparticles and deliver associated payloads such as drugs or other biomolecules (FIG. 10). These data show that cell membrane-penetrating NFs, when coated onto NPs, can improve the NP delivery to cells and subsequently, the drugs carried by the NPs, enhancing drug delivery.

The kinetics of cell uptake were measured over 4 hours to understand the time-dependent uptake in lung epithelial cells. This study can help understand dosage time required and assess therapeutic dosages needed to be delivered to the cells. NCs immediately attached to the cells within 30 minutes and later were internalized by the cells over a 24-hour period. Unlike NCs, NPs without NF coating showed a linear increase in uptake until 90 minutes and later plateaued with reduction in NP internalization (FIG. 7C). Not intending to be bound by theory, this reduction may be due to various phenomena such as exocytosis of NPs as extra cellular vehicles or other mechanisms.

NC uptake was further assessed using confocal laser scanning microscopy (CLSM) to validate the internalization. Z-stacks 3D confocal cross-sections of AT1 cells (FIG. 7D) showed rhodamine B-labeled NCs were localized next to the DAPI-stained nucleus. The x-z and y-z slices (marked with boxes in FIG. 7D) demonstrated that the nanocomposites were localized both intracellularly and extracellularly all over the treated cells, while in the nanoparticle group, the fluorescence level was much lower. The results indicated the coating with nanofibers dramatically increased both cell membrane bonding and cell uptake over a short period of time.

### Lysosome Escape of NCs

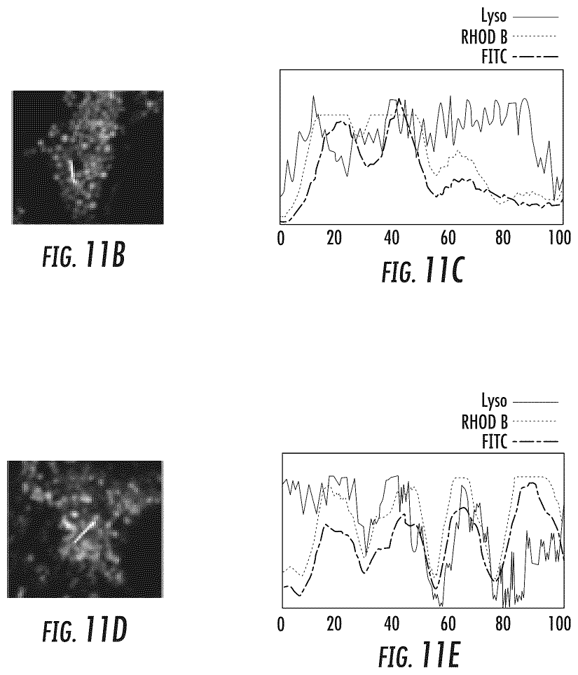

Most drugs exhibit their functions in the cytoplasm or nucleus. The ability of a nanocarrier to achieve endosome/lysosome escape can play an important role in determining their delivery efficiency. To investigate the escape of the NCs from lysosomes with various treatment times, CLSM images were analyzed to correlate the fluorescence distribution and intensity between NCs, which were constructed with FITC-labeled NFs (green) and rhodamine-labeled NCs (red), and LysoTracker™ Blue (blue). As shown in FIG. 11A, after 2 hours of incubation, a yellow fluorescence was observed on the cell membrane because of the overlay of red fluorescence from the NP and green fluorescence from the NF. This result also suggests the physical stability of the NCs in the cellular microenvironment with the NF physically attached onto the NPs. With the increase of incubation time to 8 hours, a pink fluorescence signal was shown in the cytoplasm indicating co-localization of the lysosome and NCs (FIG. 11A-C). Upon 24 hours of incubation, a yellow-to-white fluorescence signal was predominant, which is indicative of NC escape from the lysosome, while the NFs and NPs were still physically complexed together within the NCs (FIG. 11A, D-E). An analysis of fluorescence intensity distribution shows the blue fluorescence is mostly merged with the green and red fluorescence colors at 8 hours, while at 24 hours, the green and red fluorescence was shifted and dislocated with a blue fluorescence peak. This result is additional support for the time-dependent lysosome escape of NCs.

### NC Uptake Mechanism in Lung Epithelial Cells

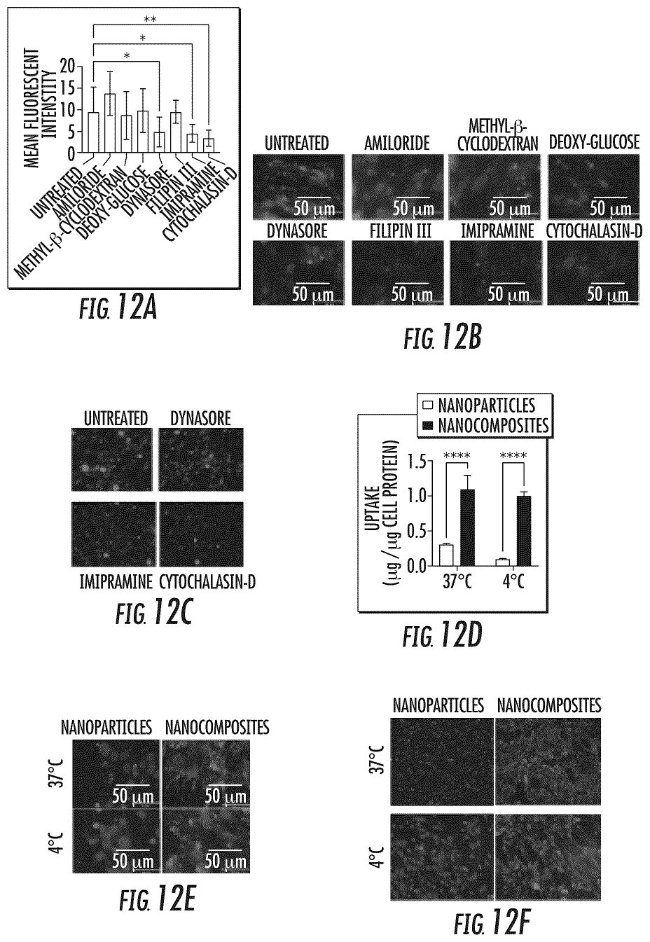

A study of the uptake mechanism in NCs can help understand the delivery efficiency and translation of results in other cell types [4]. Most NPs are internalized by cells using endocytic or phagocytic pathways, including clathrin, caveolin, micropinocytosis, and other energy independent pathways. Given the new structure and composition of NCs, NCs were screened for their uptake mechanisms using various endocytosis inhibitors. Lung epithelial cells after treatment with various endocytosis inhibitors and low temperature were treated with NCs. Similar to the free NF activity in Hela cells, macropinocytosis inhibitors of cytochalasin-D showed significant reduction in cellular uptake compared to that of untreated cells (FIG. 12A-C). Actin filaments in epithelial cells, such as Madin-Darby Canine Kidney (MDCK) kidney cells, have been shown to be critical in endocytic events where polymerization of actin filaments aids in absorption of material via macropinocytosis [46]. Cytochalasin-D inhibits the polymerization of actin filaments in epithelial cells and significantly inhibits the uptake of NCs, which was also previously reported in A549 cells [47]. This shows that NCs are uptaken by macropinocytosis by the lung epithelial cells, which were previously shown to be capable of undergoing macropinocytosis [48]. Although, among the various macropinocytosis inhibitors, some of them did not show a significant difference to the untreated cells, which shows the varied effects of inhibitors in various cell lines [4]. Similarly, lung epithelial cells were incubated at 40° C. prior to the addition of NCs to assess the energy dependency on uptake. Interestingly, cellular uptake of NCs was significantly higher than NPs showing a mixed energy-independent cell uptake mechanism along with macropinocytosis under physiological conditions (FIG. 12D-F). Taken together with cellular uptake studies, the results show that NF coating allows the NCs to immediately attach to the cell membrane and slowly aids NPs to enter cells via macropinocytosis or passive cell membrane passage in a non-energy dependent fashion.

### Mucus Permeation of NCs

Penetration of the mucus layer is highly desired to reach the injured epithelium in lungs due to the diseased state including infections and fibrotic conditions, among others. Especially in the case of lung infections, mucus hypersecretion is observed because of an increase in inflammatory signaling [49]. Adhesion of NPs to the mucus fiber is a challenge, and control of the size of the NP can improve the permeation. Here, the mucus permeation of NPs with and without the NF coating was assessed using a simulated mucus layer (FIG. 13A-C). NCs labeled with rhodamine B were layered on top of simulated mucus, and their permeation through mucus and a 0.4 μm pore size transwell into the lower chamber was recorded to assess permeation kinetics. NCs traversed at a significantly higher rate compared to NPs until 8 hours, and later, the permeation rate was similar to NP permeation with and without the NF coating as determined using simulated kinetics (FIG. 13A-C). Overall, more NCs permeated through the mucus than the NP alone, showing that NFs may interfere with NP binding to mucin proteins.

### Translative Potential of NC's

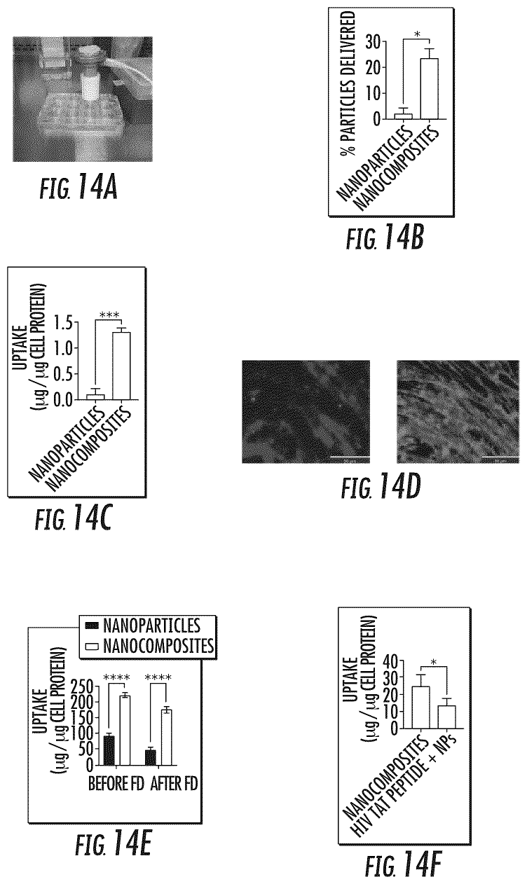

The stability in storage and nebulization for their potential in pulmonary drug delivery was assessed. The freeze-dried NCs still showed significantly higher uptake in lung epithelial cells compared to NPs (FIG. 14D) demonstrating the storage ability of NCs in powder form. NCs also showed higher uptake compared to a conventional cell membrane penetrating HIV Tat peptide coated nanoparticles (FIG. 14E-F). This indicates the superior ability of the NFs to improve NP affinity towards the cell membrane. NCs in powder form have been shown to possess higher stability after reaction compared to NPs, suggesting stability of the NF binding of NPs after undergoing freeze drying and storage at −20° C. To assess the abilities of NFs to improve the NP uptake in cells when nebulized, NCs and NPs were delivered to lung AT1 cells in vitro via a lab module nebulizer generating droplets of size 2.5-4 μm (FIG. 14A) [54]. NCs delivered via nebulization showed improved uptake with deposition efficiency over 23% delivered compared to 3% of nanoparticles (FIG. 14B-C). Low retention of NPs compared to NCs can be explained by the higher negative charge, which may pose an interaction issue with the material used for delivering particles to the well plate and also loss of samples during nebulization. The amount of NCs that reach the media after loss during nebulization have higher affinity for the cell membrane and are taken in at a higher rate. Nebulization studies in vitro show that NCs delivered via inhalation have the potential to enhance pulmonary drug delivery compared to PLGA-based NPs.

### D. Conclusion

This example demonstrates that using NFs made from de novo self-assembled peptides and coating them on PLGA polymer nanoparticles can improve the uptake of the produced nanocomposites in lung epithelial cells in vitro. The NF coatings effectively improve nanocomposite uptake in macrophages along with endothelial cells. Both cell lines respond to bacterial pathogens and have high activity in lung diseases such as infections. In addition, the NCs can be delivered across a simulated mucus layer, showing the ability to permeate the mucus layer effectively.

### E. References

[1] M. J. Mitchell, M. M. Billingsley, R. M. Haley, M. E. Wechsler, N. A. Peppas, R. Langer, Engineering precision nanoparticles for drug delivery, Nature Reviews Drug Discovery 20 (2) (2021) 101-124.
[2] S. Gelperina, K. Kisich, M. D. Iseman, L. Heifets, The potential advantages of nanoparticle drug delivery systems in chemotherapy of tuberculosis, Am J Respir Crit Care Med 172 (12) (2005) 1487-1490.
[3] P. J. Oh N, Endocytosis and exocytosis of nanoparticles in mammalian cells, Int J Nanomedicine 9 (2014) 51-63.
[4] J. J. Rennick, A. P. R. Johnston, R. G. Parton, Key principles and methods for studying the endocytosis of biological and nanoparticle therapeutics, Nature Nanotechnology 16 (3) (2021) 266-276.
[5] Y. Abo-Zeid, G. R. Williams, L. Touabi, G. R. McLean, An investigation of rhinovirus infection on cellular uptake of poly(glycerol-adipate) nanoparticles, Int J Pharm 589 (2020) 119826.
[6] A. Calvo, E. Moreno, U. Clemente, E. Pérez, E. Larrea, C. Sanmartin, J. M. Irache, S. Espuelas, Changes in the nanoparticle uptake and distribution caused by an intramacrophagic parasitic infection, Nanoscale 13 (41) (2021) 17486-17503.
[7] A. R. Kirtane, M. Verma, P. Karandikar, J. Furin, R. Langer, G. Traverso, Nanotechnology approaches for global infectious diseases, Nature Nanotechnology 16 (4) (2021) 369-384.
[8] P. Vila-Gómez, J. E. Noble, M. G. Ryadnov, Peptide Nanoparticles for Gene Packaging and Intracellular Delivery, Methods Mol Biol 2208 (2021) 33-48.
[9] W.-j. Jeong, J. Bu, L. J. Kubiatowicz, S. S. Chen, Y. Kim, S. Hong, Peptide-nanoparticle conjugates: a next generation of diagnostic and therapeutic platforms?, Nano Convergence 5 (1) (2018) 38.
[10] I. Gessner, I. Neundorf, Nanoparticles Modified with Cell-Penetrating Peptides: Conjugation Mechanisms, Physicochemical Properties, and Application in Cancer Diagnosis and Therapy, Int J Mol Sci 21 (7) (2020).
[11] N. G. S. Boussoufi F, Chang R, Webster T J., Synthesis and study of cell-penetrating peptide-modified gold nanoparticles, Int J Nanomedicine 13 (2018) 6199-6205.
[12] Y. Li, L. Hao, F. Liu, L. Yin, S. Yan, H. Zhao, X. Ding, Y. Guo, Y. Cao, P. Li, Z. Wang, H. Ran, Y. Sun, <p> Cell penetrating peptide-modified nanoparticles for tumor targeted imaging and synergistic effect of sonodynamic/HIFU therapy</p>, International Journal of Nanomedicine Volume 14 (2019) 5875-5894.
[13] H. L. Huang, W. J. Lin, Dual Peptide-Modified Nanoparticles Improve Combination Chemotherapy of Etoposide and siPIK3CA Against Drug-Resistant Small Cell Lung Carcinoma, Pharmaceutics 12 (3) (2020).
[14] H. Cui, M. J. Webber, S. I. Stupp, Self-assembly of peptide amphiphiles: From molecules to nanostructures to biomaterials, Peptide Science 94 (1) (2010) 1-18.
[15] C. A. Hauser, S. Zhang, Designer self-assembling peptide nanofiber biological materials, Chemical Society Reviews 39 (8) (2010) 2780-2790.
[16] J. S. Rudra, Y. F. Tian, J. P. Jung, J. H. Collier, A self-assembling peptide acting as an immune adjuvant, Proceedings of the National Academy of Sciences 107 (2) (2010) 622-627.
[17] C. Yan, D. J. Pochan, Rheological properties of peptide-based hydrogels for biomedical and other applications, Chemical Society Reviews 39 (9) (2010) 3528-3540.
[18] M. C. Branco, D. M. Sigano, J. P. Schneider, Materials from peptide assembly: towards the treatment of cancer and transmittable disease, Current Opinion in Chemical Biology 15 (3) (2011) 427-434.
[19] G. A. Hudalla, T. Sun, J. Z. Gasiorowski, H. Han, Y. F. Tian, A. S. Chong, J. H. Collier, Gradated assembly of multiple proteins into supramolecular nanomaterials, Nature Materials 13 (2014) 829-834.
[20] V. A. Kumar, N. L. Taylor, S. Shi, B. K. Wang, A. A. Jalan, M. K. Kang, N. C. Wickremasinghe, J. D. Hartgerink, Highly angiogenic peptide nanofibers, ACS Nano 9 (1) (2015) 860-868.
[21] A. N. Moore, J. D. Hartgerink, Self-assembling multidomain peptide nanofibers for delivery of bioactive molecules and tissue regeneration, Accounts of Chemical Research 50 (4) (2017) 714-722.
[22] Y.-A. Lin, A. G. Cheetham, P. Zhang, Y.-C. Ou, Y. Li, G. Liu, D. Hermida-Merino, I. W. Hamley, H. Cui, Multiwalled Nanotubes Formed by Catanionic Mixtures of Drug Amphiphiles, ACS Nano 8 (12) (2014) 12690-12700.
[23] D. M. Raymond, B. L. Nilsson, Multicomponent peptide assemblies, Chemical Society Reviews 47 (10) (2018) 3659-3720.
[24] X. Du, J. Zhou, J. Shi, B. Xu, Supramolecular Hydrogelators and Hydrogels: From Soft Matter to Molecular Biomaterials, Chemical Reviews 115 (24) (2015) 13165-13307.
[25] H. Acar, S. Srivastava, E. J. Chung, M. R. Schnorenberg, J. C. Barrett, J. L. LaBelle, M. Tirrell, Self-assembling peptide-based building blocks in medical applications, Advanced Drug Delivery Reviews 110-111 (2017) 65-79.
[26] L. Adler-Abramovich, E. Gazit, The physical properties of supramolecular peptide assemblies: from building block association to technological applications, Chemical Society Reviews 43 (20) (2014) 6881-6893.
[27] S. Fleming, R. V. Ulijn, Design of nanostructures based on aromatic peptide amphiphiles, Chemical Society Reviews 43 (23) (2014) 8150-8177.
[28] B. H. San, J. Hwang, S. Sampath, Y. Li, L. L. Bennink, S. M. Yu, Self-Assembled Water-Soluble Nanofibers Displaying Collagen Hybridizing Peptides, Journal of the American Chemical Society 139 (46) (2017) 16640-16649.
[29] S. Yang, H. Dong, Modular design and self-assembly of multidomain peptides towards cytocompatible supramolecular cell penetrating nanofibers, RSC Advances 10 (49) (2020) 29469-29474.
[30] D. Xu, D. Dustin, L. Jiang, D. S. K. Samways, H. Dong, Designed filamentous cell penetrating peptides: probing supramolecular structure-dependent membrane activity and transfection efficiency, Chemical Communications 51 (59) (2015) 11757-11760.
[31] D. Xu, L. Jiang, L. DeRidder, B. Elmore, M. Bukhari, Q. Wei, D. S. K. Samways, H. Dong, Membrane activity of a supramolecular peptide-based chemotherapeutic enhancer, Molecular BioSystems 12 (9) (2016) 2695-2699.
[32] D. Xu, D. S. K. Samways, H. Dong, Fabrication of self-assembling nanofibers with optimal cell uptake and therapeutic delivery efficacy, Bioactive Materials 2 (4) (2017) 260-268.
[33] D. Xu, L. DeRidder, B. Elmore, H. Dong, Self-assembly of Filamentous Cell Penetrating Peptides for Gene Delivery, Peptide Self-Assembly: Methods and Protocols2018, pp. 271-281.
[34] S. Yang, D. Xu, H. Dong, Design and fabrication of reduction-sensitive cell penetrating nanofibers for enhanced drug efficacy, Journal of Materials Chemistry B 6 (44) (2018) 7179-7184.
[35] F. Emami, S. J. Mostafavi Yazdi, D. H. Na, Poly(lactic acid)/poly(lactic-co-glycolic acid) particulate carriers for pulmonary drug delivery, Journal of Pharmaceutical Investigation 49 (4) (2019) 427-442.
[36] O. Harush-Frenkel, M. Bivas-Benita, T. Nassar, C. Springer, Y. Sherman, A. Avital, Y. Altschuler, J. Borlak, S. Benita, A safety and tolerability study of differently-charged nanoparticles for local pulmonary drug delivery, Toxicol Appl Pharmacol 246 (1-2) (2010) 83-90.
[37] J. U. Menon, P. Ravikumar, A. Pise, D. Gyawali, C. C. W. Hsia, K. T. Nguyen, Polymeric nanoparticles for pulmonary protein and DNA delivery, Acta Biomaterialia 10 (6) (2014) 2643-2652.
[38] K. Ohashi, T. Kabasawa, T. Ozeki, H. Okada, One-step preparation of rifampicin/poly(lactic-co-glycolic acid) nanoparticle-containing mannitol microspheres using a four-fluid nozzle spray drier for inhalation therapy of tuberculosis, J Control Release 135 (1) (2009) 19-24.
[39] M. Jerabek-Willemsen, T. Andre, R. Wanner, H. M. Roth, S. Duhr, P. Baaske, D. Breitsprecher, MicroScale Thermophoresis: Interaction analysis and beyond, Journal of Molecular Structure 1077 (2014) 101-113.
[40] A. Chatterjee, D. K. Mandal, Denaturant-induced equilibrium unfolding of concanavalin A is expressed by a three-state mechanism and provides an estimate of its protein stability, Biochimica et Biophysica Acta (BBA)-Proteins and Proteomics 1648 (1) (2003) 174-183.
[41] Z. Huang, S. N. Kłodzińska, F. Wan, H. M. Nielsen, Nanoparticle-mediated pulmonary drug delivery: state of the art towards efficient treatment of recalcitrant respiratory tract bacterial infections, Drug Delivery and Translational Research 11 (4) (2021) 1634-1654.
[42] F. Andrade, D. Rafael, M. Videira, D. Ferreira, A. Sosnik, B. Sarmento, Nanotechnology and pulmonary delivery to overcome resistance in infectious diseases, Adv Drug Deliv Rev 65 (13-14) (2013) 1816-27.
[43] M. Pivard, K. Moreau, F. Vandenesch, Staphylococcus aureus Arsenal To Conquer the Lower Respiratory Tract, mSphere 6 (3) (2021) e00059-21.
[44] K. Saar, M. Lindgren, M. Hansen, E. Eiriksdóttir, Y. Jiang, K. Rosenthal-Aizman, M. Sassian, U. Langel, Cell-penetrating peptides: a comparative membrane toxicity study, Anal Biochem 345 (1) (2005) 55-65.
[45] E. Trofimenko, G. Grasso, M. Heulot, N. Chevalier, M. A. Deriu, G. Dubuis, Y. Arribat, M. Serulla, S. Michel, G. Vantomme, F. Ory, L. C. Dam, J. Puyal, F. Amati, A. Lüthi, A. Danani, C. Widmann, Genetic, cellular, and structural characterization of the membrane potential-dependent cell-penetrating peptide translocation pore, eLife 10 (2021) e69832.
[46] T. A. Gottlieb, I. E. Ivanov, M. Adesnik, D. D. Sabatini, Actin microfilaments play a critical role in endocytosis at the apical but not the basolateral surface of polarized epithelial cells, J Cell Biol 120 (3) (1993) 695-710.
[47] D. A. Kuhn, D. Vanhecke, B. Michen, F. Blank, P. Gehr, A. Petri-Fink, B. Rothen-Rutishauser, Different endocytotic uptake mechanisms for nanoparticles in epithelial cells and macrophages, Beilstein J Nanotechnol 5 (2014) 1625-36.
[48] X. P. Lin, J. D. Mintern, P. A. Gleeson, Macropinocytosis in Different Cell Types: Similarities and Differences, Membranes (Basel) 10 (8) (2020).
[49] M. Zanin, P. Baviskar, R. Webster, R. Webby, The Interaction between Respiratory Pathogens and Mucus, Cell Host & Microbe 19 (2) (2016) 159-168.
[50] S. K. Lai, Y.-Y. Wang, J. Hanes, Mucus-penetrating nanoparticles for drug and gene delivery to mucosal tissues, Advanced Drug Delivery Reviews 61 (2) (2009) 158-171.
[51] S. Hua, M. B. C. de Matos, J. M. Metselaar, G. Storm, Current Trends and Challenges in the Clinical Translation of Nanoparticulate Nanomedicines: Pathways for Translational Development and Commercialization, Frontiers in Pharmacology 9 (2018).
[52] M. Z. Rahman Sabuj, N. Islam, Inhaled antibiotic-loaded polymeric nanoparticles for the management of lower respiratory tract infections, Nanoscale Advances 3 (14) (2021) 4005-4018.
[53] S. P. Newman, Drug delivery to the lungs: challenges and opportunities, Therapeutic Delivery 8 (8) (2017) 647-661.
[54] J. C. Horstmann, C. R. Thorn, P. Carius, F. Graef, X. Murgia, C. de Souza Carvalho-Wodarz, C.-M. Lehr, A Custom-Made Device for Reproducibly Depositing Pre-metered Doses of Nebulized Drugs on Pulmonary Cells in vitro, Frontiers in Bioengineering and Biotechnology 9 (2021).
[55] V. L. Messerschmidt, U. Chintapula, A. E. Kuriakose, S. Laboy, T. T. D. Truong, L. A. Kydd, J. Jaworski, Z. Pan, H. Sadek, K. T. Nguyen, J. Lee, Notch Intracellular Domain Plasmid Delivery via Poly(Lactic-Co-Glycolic Acid) Nanoparticles to Upregulate Notch Pathway Molecules, Front Cardiovasc Med 8 (2021) 707897-707897.
[56] R. Iyer, T. Nguyen, D. Padanilam, C. Xu, D. Saha, K. T. Nguyen, Y. Hong, Glutathione-responsive biodegradable polyurethane nanoparticles for lung cancer treatment, Journal of Controlled Release 321 (2020) 363-371.
[57] B. Casciaro, I. d'Angelo, X. Zhang, M. R. Loffredo, G. Conte, F. Cappiello, F. Quaglia, Y.-P. P. Di, F. Ungaro, M. L. Mangoni, Poly(lactide-co-glycolide) Nanoparticles for Prolonged Therapeutic Efficacy of Esculentin-la-Derived Antimicrobial Peptides against Pseudomonas aeruginosa Lung Infection: in Vitro and in Vivo Studies, Biomacromolecules 20 (5) (2019) 1876-1888.

### EXAMPLE 2

### Nanoparticles for Drug Delivery

This Example describes the characterization of nanoparticles for use in drug delivery systems. PLGA nanoparticles were synthesized as described in Example 1. Nanofibers were synthesized as described in Example 1 with the primary sequences indicated for each group in Table 2. PLGA nanoparticles were coated as described in Example 1. PLGA nanoparticles without nanofibers (plain PLGA NPs), PLGA nanoparticles coated with K₉ nanofibers (SEQ ID NO: 2) (K₉-PLGA NPs), and PLGA nanoparticles coated with K₁₀ nanofibers (SEQ ID NO: 3) (K₁₀-PLGA NPs) were synthesized.

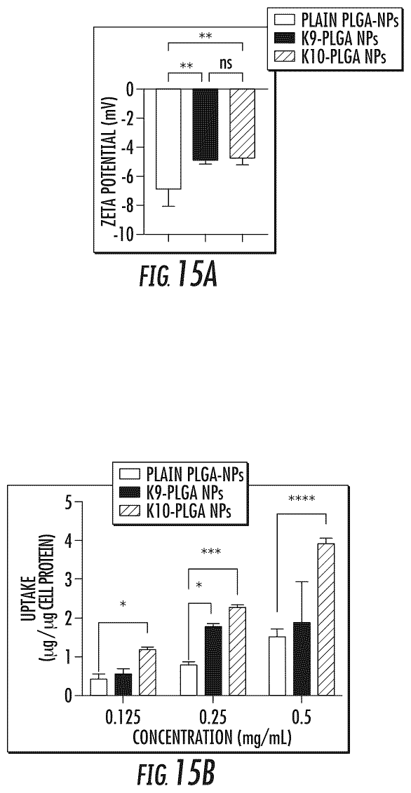

A ZETAPALS90 dynamic light scattering (DLS) detector (Brookhaven Instrument, Holtsville, NY) was used to determine the size and charge of the nanoparticles. For DLS measurements, 50 μL of 1 mg/mL nanoparticle suspension was mixed with 3 mL of DI water in a transparent cuvette and placed in the instrument to measure size, while a DLS probe was used to measure the zeta potential of the nanoparticles. DLS measurements are shown in Table 2. The zeta potential for each nanoparticle group is shown in FIG. 15A.

TABLE 2
DLS measurements for each nanoparticle group.

| Nanoparticle Group | DLS Size (nm) |
|---|---|
| Plain PLGA NPs | 164 ± 64 |
| K₉-PLGA NPs | 177 ± 16 |
| K₁₀-PLGA NPs | 195 ± 15 |

The uptake of PLGA nanoparticles without nanofibers, PLGA nanoparticles coated with K₉ nanofibers (SEQ ID NO: 2), and PLGA nanoparticles coated with K₁₀ nanofibers (SEQ ID NO: 3) was measured (FIG. 15B). Nanoparticle concentrations at 0.125 mg/mL, 0.25 mg/mL, and 0.5 mg/mL were used. Uptake was normalized to total cell protein. Uptake was increased by the coating with the peptide nanofibers. The highest uptake ability was with K₁₀-PLGA NPs.

### EXAMPLE 3

Conjugation Efficiency of the Coating of PLGA Nanoparticles with Nanofibers

In this Example, the conjugation efficiency of PLGA nanoparticles and synthesized nanofibers were assessed. PLGA nanoparticles were synthesized as described in Example 1. Nanofibers were synthesized as described in Example 1 with the primary sequences indicated for each group in Table 3. PLGA nanoparticles were coated as described in Example 1 using the total mass of nanofiber used indicated in Table 2 and 2 mg of PLGA nanoparticles. The conjugation efficiency was measured. It was found that for K₉As (QW)₆, conjugation efficiency was slightly improved at 0.25 mg total mass of nanofiber used and that K₁₀(QW)₆E₃ had overall better conjugation efficiency, especially at 0.25 mg total mass of nanofiber used.

TABLE 3
Conjugation efficiency of nanofibers
with different peptide sequences.

| | Total mass of | Conjugation |
|---|---|---|
| Nanofiber group | nanofiber used (mg) | efficiency |
| K₉A₅(QW)₆ (SEQ ID NO: 5) | 0.25 | 25.5% |
| K₉A₅(QW)₆ (SEQ ID NO: 5) | 0.50 | 24.7% |
| K₁₀(QW)₆E₃ (SEQ ID NO: 1) | 0.25 | 37.4% |
| K₁₀(QW)₆E₃(SEQ ID NO: 1) | 0.50 | 28.7% |

### EXAMPLE 4

### Antimicrobial Nanocomposites for the Treatment of MRSA Lung Infections

### A. Introduction

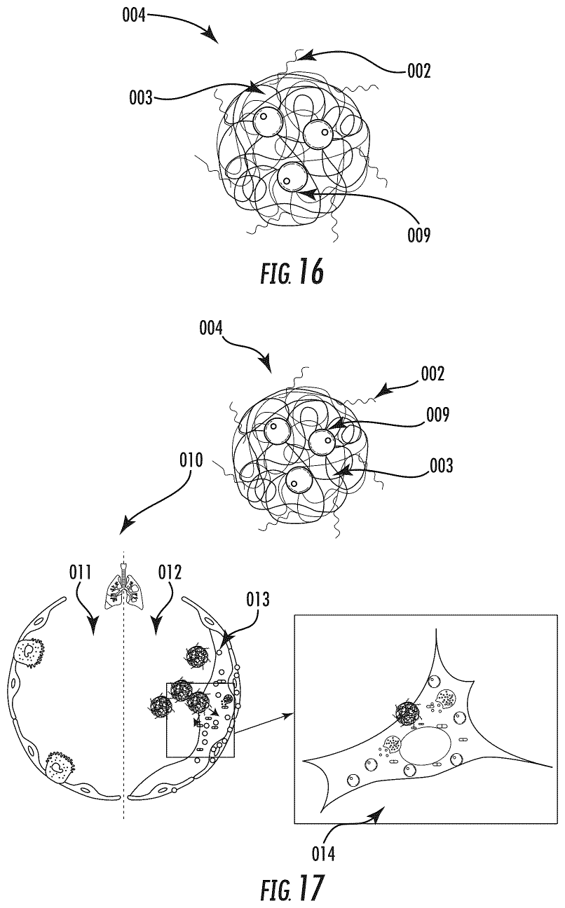

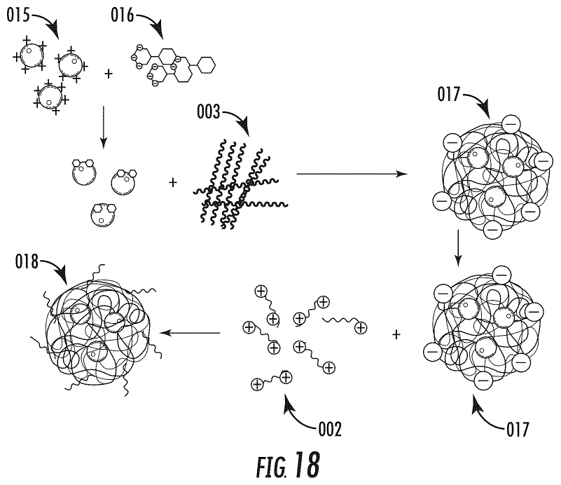

Lung infections such as MRSA have been on the rise in recent years, and there is a need for more effective treatment. In this Example, the therapeutic efficacy to treat MRSA-infect cells in vitro and biodistribution in vivo of antimicrobial nanocomposites (AMNCs) is shown, and the ability of AMNCs to act as a drug carrier to inhibit MRSA infection in primary lung alveolar epithelial cells is displayed. FIG. 16 shows a schematic of an AMNC (004). In this embodiment, nanofibers (002) are bound to PLGA polymer (003), which has bound a payload (009). In this Example, the payload comprises rhodamine B for dye for characterization and vancomycin for MRSA treatment. FIG. 17 is a schematic of a drug delivery system of AMNCs for the treatment of S. aureus. Nanofibers (002) are bound to PLGA polymer (003), which has bound a payload (009) comprising the drug vancomycin. When the nanocomposites are inhaled (010) into the healthy (011) or diseased (012) alveoli, which contain S. aureus (013) and the nanocomposites enter the diseased alveoli of the lungs, specifically lung epithelial cells (AT1 cells) (014). S. aureus death results from intracellular vancomycin release.

### B. Synthesis of Antimicrobial Nanoparticles and Nanocomposites

In FIG. 18, a schematic of the synthesis of antimicrobial nanoparticles and AMNCs is displayed. A double emulsion method was used to produce antimicrobial nanoparticles and AMNCs. 50 mg of vancomycin hydrochloride (015) was dissolved in 0.5 mL of deionized (DI) water with 3 mg of dextran sulfate (016) added to the solution. 100 mg of PLGA (003) was dissolved in 4 mL of DCM. The vancomycin solution was emulsified with the PLGA solution by sonicating at 40% power of the device with 2 cycles, 1 minute on and 30 seconds off. Later, the primary emulsion was added dropwise into 5% (w/v) PVA under stirring. A second emulsion was creating by sonicating the above solution using a Branson sonicator at 25 Amp with 4 cycles (1 minute on and 30 seconds off). The second emulsion was left stirring at room temperature to remove the DCM solvent. The nanoparticles (017) formed were collected via washing and centrifugation and freeze dried until dry.

Antimicrobial nanoparticles of 2 mg loaded with vancomycin were resuspended in Tris buffer and mixed with 0.5 mg nanofibers for an hour at room temperature. Nanofibers were synthesized as previously described. Later, the nanocomposites formed by the coating of nanofibers onto nanoparticles (018) were collected via centrifugation at 15,000 rpm for 7 minutes at 40° C. The nanocomposite pellet was then re-suspended in various buffers or media as needed for the experiments.

### C. Characterization of AMNCs

### Morphology of AMNCs

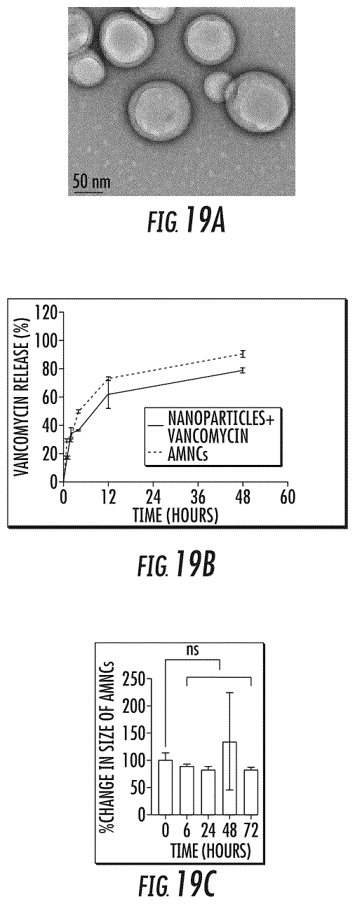

The morphology of AMNCs loaded with vancomycin was observed. Briefly, AMNCs were dropped onto copper grids, and the excess AMNC suspension was removed. Samples of AMNCs on the copper grid were imaged with a high-resolution TEM (Hitachi H-9500) (FIG. 19A). The AMNCs showed spherical morphology of approximately 200 nm diameter, which is suitable for lower tract drug delivery.

### Drug Release Profiles

The drug release profile of vancomycin from antimicrobial nanoparticles and AMNCs was observed at pH 7.4 over 48 hours (Imipramine). Briefly, triplicate samples of 3 mg antimicrobial nanoparticles and AMNCs were dispersed in 1 mL of PBS (7.4) and dialyzed against a 3 kDa tubing with a sink reservoir volume of 10 mL. At every timepoint up to 48 hours, 1 mL of reservoir volume was collected and replaced with fresh PBS. After collecting samples, a protein quantification assay (BCA) was used to assess vancomycin release from collected samples by using a vancomycin standard. Both antimicrobial nanoparticles and AMNCs showed a bi-phasic drug release with an initial burst release and a sustained release in PBS at 37° C. AMNCs showed a slightly higher rate of vancomycin release after 4 hours, but over 48 hours, antimicrobial nanoparticles and AMNCs showed a similar amount of vancomycin release.

### AMNC Stability

The stability of AMNCs in saline was observed over 72 hours using dynamic light scattering, and the % change in size of the AMNCs was observed (FIG. 19C). AMNCs were stable in saline over 72 hours, and the % change in size was not significant over 72 hours. Saline stability can be translated to nebulization and other temporary storage before administration.

### D. Cytocompatibility of AMNCs

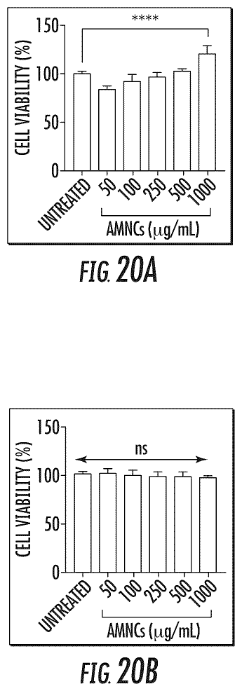

To test the cytocompatibility of AMNCs, two different epithelial cell types residing in the lower respiratory tract were utilized, AT1 cells and A549 cells. Both AT1 and A549 cells were seeded at confluency and allowed to attach overnight. The next day, various concentrations of AMNCs were given to the cells by replacing the culture media with fresh media containing the particles. After 72 hours, the cells were washed 3 times with PBS and given MTS reagent to assess the cell viability. The absorbance from the cells after adding the MTS reagent was recorded using a plate reader (Tecan) and plotted in GraphPad Prism. The data for the treated groups were normalized against the untreated groups. The results indicated that the AMNCs were cytocompatible for AT1 cells and A549 cells at concentrations up to 1000 μg/mL following 72 hours of incubation (FIG. 20A-B). More than 90% of cells at all concentrations showed viability, indicating that the AMNCs are relatively non-toxic. Not intending to be bound by theory, the nanofiber coating reduces AMNC toxicity, making AMNCs cytocompatible.

### E. Nanocomposite Uptake by Infected Cells

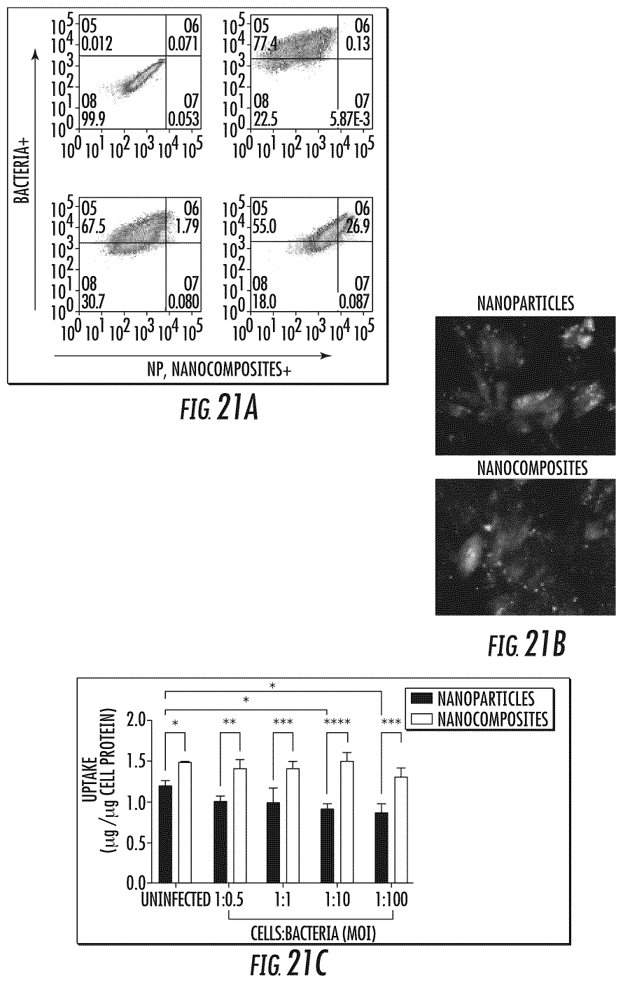

Nanocomposite and nanoparticle uptake by infected cells was measured via flow cytometry and quantified using fluorescence techniques. Briefly, AT1 cells were seeded at confluency in a 24-well plate. After overnight culture, the cells were treated with overnight cultured MRSA bacteria at various ratios (cell: bacteria; 1:0.5, 1:1, 1:10, 1:100). The co-culture of cells and bacteria were spun down at 2,000 rpm for 5 minutes. After centrifugation, the cells were incubated with bacteria for 4 hours for infection, later washed 3 times with PBS, and treated with 100 μg/mL of gentamycin to remove the extracellular bacteria. Nanoparticles and nanocomposites (nanoparticles with nanofiber coating) were added to the infected cells at 0.5 μg/mL and incubated for 90 minutes. After 90 minutes, the cells were washed with PBS three times and stained with NucBlue (ThermoFischer) for visualizing the nucleus. MRSA was stained with SYTO 9 gated on the y-axis and AMNCs were stained with Rhodamine B gated on the x-axis on the resulting flow cytometry dot plots (FIG. 21A). Images were taken to observe cell uptake using a fluorescent microscope (ECHO, CA) (FIG. 21B).

Nanocomposite uptake by AT1 cells infected with MRSA was also assessed using fluorescence imaging. AT1 cells were seeded into 24-well plates at confluency and the next day, infected with MRSA at a multiplicity of infection (MOI) ranging from 0.5-100. Polybrene, a transfection reagent, was used as a control to enhance the uptake of nanoparticles and AMNCs. The cells, along with the bacteria, were spun down for 5 min at 2000 rpm to increase the bacterial uptake by the cells. After 4 hours, the cells were washed and treated with 100 μg/mL of gentamycin for 30 minutes to remove the extracellular bacteria. After treatment, the cells were washed and treated with 0.5 mg/mL of rhodamine B-loaded nanoparticles for 90 minutes. Later, the cells were washed three times with PBS and lysed using 1% Triton-X. The cell lysate was used to analyze the fluorescence of the nanoparticles and AMNCs and protein content from the cells.

When infected with various MOI (0.5, 1, 10, 100) of MRSA, AT1 cells showed a reduction in nanoparticle uptake (FIG. 21C). Not intending to be bound by theory, this reduction in uptake of nanoparticles may be due to alterations in various uptake endocytosis mechanisms. Compared to nanoparticles, nanocomposites showed significantly higher uptake at all MOI levels of MRSA infection in AT1 cells

### F. Antimicrobial Properties of AMNCs

### Minimum Inhibitory Concentration Assay

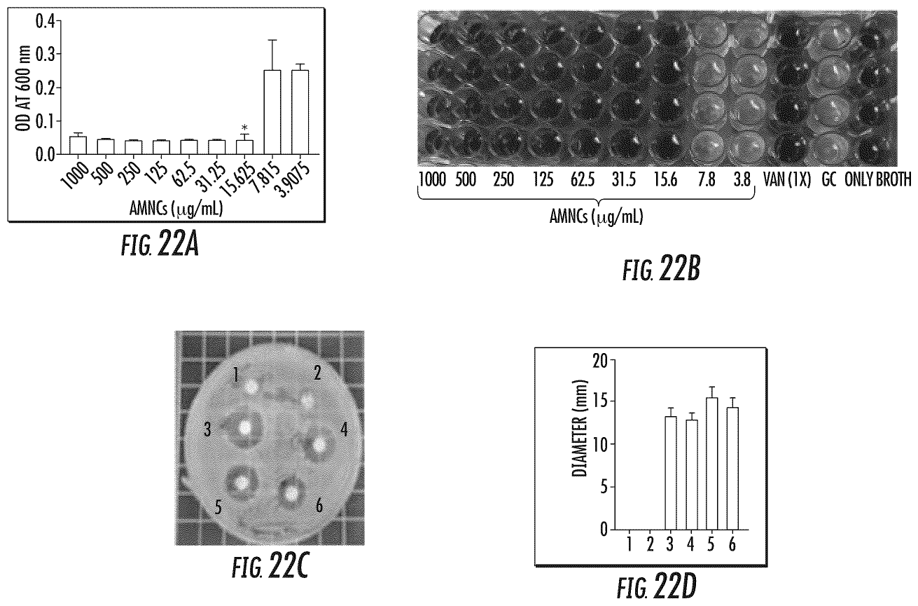

A MRSA colony was picked and grown in BHI media overnight. The next day, MRSA was diluted to 1×10⁶ CFU/mL for testing the MIC of AMNCs. Briefly, AMNCs were serially diluted in BHI media to various concentrations (1.5-1000 μg/mL), along with only BHI media, free vancomycin at 2 μg/mL (1X Minimum Inhibitory Concentration, MIC; positive control), and BHI media with MRSA only (negative control). 1×10⁶ CFU/mL was mixed with various concentrations of AMNCs at 1:1 ratio and incubated at 37° C. for 24 hours. After 24 hours, 0.015% resazurin was added for colorimetric assessment of bacterial inhibition, and the samples were incubated further for 1 hour. The color was assessed by a plate reader at 600 nm (FIG. 22A). A purple color indicates bacterial inhibition, while pink indicates bacterial growth (FIG. 22B). The results indicated that AMNCs inhibited MRSA growth. The data were normalized against the controls to determine the MIC value, which was 15.6 μg/mL, similar to free vancomycin.

### Zone of Inhibition Studies

Zone of inhibition studies were performed to assess the antimicrobial potential of AMNCs in comparison to free vancomycin. Briefly, 200 μL of 1×10⁸ CFU/mL of MRSA bacteria were plated onto BHI agar plates. 7-mm sterile discs were loaded with 50 μL of (1) only BHI media, (2) plain NPs, (3) 2X MIC of free vancomycin, (4) 1X MIC of free vancomycin, (5) 2X MIC of vancomycin-loaded nanoparticles, and (6) 1X MIC of vancomycin-loaded nanoparticles The loaded discs were placed on the MRSA plated agar plates in triplicate and incubated for 24 hours. Later, pictures were taken of each disc with scale to measure the diameter of the zone of inhibition of bacterial growth (FIG. 22C). The zone of inhibition diameter was measured for each condition using the ImageJ software and plotted (FIG. 22D). The zone of inhibition study showed similar diameters for zones of inhibition between 1X and 2X MIC concentrations of free vancomycin and equivalent concentrations of AMNCs. Nanoparticles without vancomycin did not show inhibit MRSA bacterial growth on the plates.

### Intracellular Killing Efficacy of AMNCs

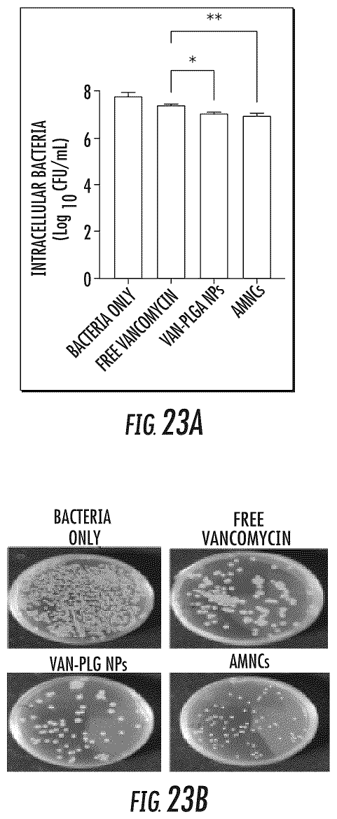

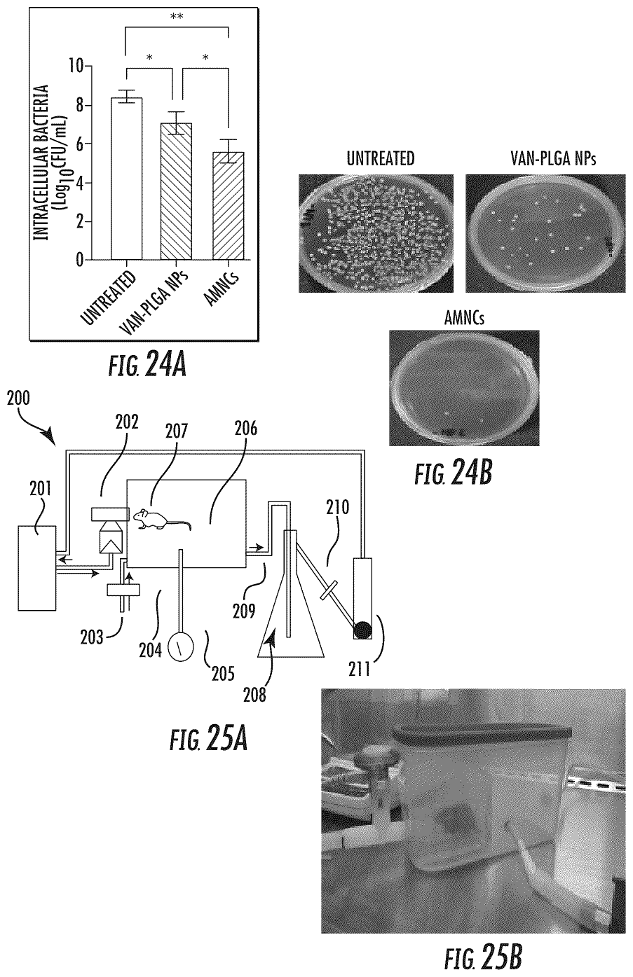

To assess the intracellular killing efficacy of AMNCs, an intracellular killing study was performed. AT1 cells were seeded at confluency in a 24-well plate and allowed to attach overnight. The next day, a ratio of 1:10 cells to MRSA was given to the cells via re-suspension in AT1 cell growth media, followed by centrifugation to facilitate bacterial infection of the exposed cells. After 3 hours, the cells were washed with PBS and incubated for 30 minutes with gentamycin at a concentration of 100 μg/mL to remove the added bacteria. The cells were washed and then treated with 0.5 mg/mL of free vancomycin, vancomycin-loaded nanoparticles, or AMNCs for 12 hours. The concentration of the free drug is equivalent to the drug loaded into nanoparticles. After 12 hours, cells were washed three times with PBS and lysed with either DI water or 0.02% Triton X-100. Serially diluted cell lysate was plated onto BHI agar plates to quantify the number of intracellular MRSA bacteria. After 14-18 hours, the plates were imaged, the colonies formed on the agar plates were counted, and the data were plotted (FIG. 23A-B).

Cell lysate from AT1 cells infected with MRSA showed a reduction in the bacterial burden in the vancomycin groups. Out of all the vancomycin-treated groups, AMNCs showed a higher inhibition of intracellular MRSA compared to the group treated with nanoparticles without a nanofiber coating or the free vancomycin group. Gentamycin treatment of infected cells before giving any treatments ensured removal of the extracellular bacteria and focused the treatment on the intracellular MRSA. After 12 hours of incubation with nanoparticles, without intending to be bound by theory, AMNCs and free vancomycin showed that the higher uptake ability of AMNCs can improve the antibiotic payload delivery in infected cells evident from the reduction in MRSA colonies from cell lysate.

### In Vitro Nebulization

In vitro nebulization was performed to assess the therapeutic efficacy of the nebulized AMNCs in vitro. AT1 cells were seeded at confluency in a 12-well plate and allowed to attach overnight. The next day, AT1 cells were infected at 1:10 cells to MRSA. Next, 1 mg each of nanoparticles loaded with vancomycin and AMNCs were loaded into a lab module nebulizer (Aeroneb®) along with equivalent amounts of free vancomycin dissolved in 1 mL of PBS. The above treatment groups were nebulized on top of the cells using a 24-well transwell insert with the membrane removed to facilitate airflow exit for the nebulized particles. After treatment for 12 hours, the cells were washed with PBS and lysed with DI water. Serially diluted cell lysate was plated onto a BHI agar plate. After 14-18 hours, the colonies formed on the agar plates were counted, and the plates were imaged (FIG. 23B). The log₁₀ CFU was calculated from each well to determine the intracellular bacteria level (FIG. 23A). AMNCs reduced the intracellular bacteria with over one log₁₀ reduction compared to nanoparticles loaded with vancomycin. These results show that nebulization of AMNCs do not hinder the enhanced uptake abilities of AMNCs, evident by the increased killing of MRSA.

### G. In Vivo Biodistribution

### Mouse Studies

To assess the targeting capability of nanocomposite formulations for the lower respiratory tract, a biodistribution study in mice was performed. For this study, 7 10-week-old C57BL/6J mice (both sexes) were used. Indocyanine green (ICG)-labeled nanoparticle and nanocomposite formulations were nebulized using an Aeroneb® lab module nebulizer in a modified closed circuit. Saline solution nebulization was used as a negative control. FIG. 25A-B shows the setup of the inhalation delivery of nebulized PLGA NPs and AMNCs in mice. FIG. 25A is a schematic view of the modified nebulizer setup (200) for inhalable delivery, and FIG. 25B is an actual image of the nebulizer setup. A pump system (201) is attached to a lab module nebulizer (202). Clean replacement air (203) enters through a HEPA filter (204). An air pressure gauge (205) is attached to the mice restrainer (206) for inhalation through the nebulizer, in which a mouse (207) is placed. A vacuum flask (208) is attached at a port to draw aerosol (209). A 0.2 μm HEPA filter (210) is attached to an airflow regulator (211), which is also attached to the lab module nebulizer (202). The apparatus with closed circuit allows for filtering nanoparticles and aerosols via the HEPA filter (204), ensuring safety for operating personnel.

ICG-loaded PLGA NPs (PLGA-NPs) and nanofiber-coated ICG-PLGA NPs (AMNCs) were re-suspended at 2.5 mg/mL in saline for nebulization. Mice were restrained in the chamber and nebulized with various groups, including a saline control, for 20 minutes. After nebulization, the mice were monitored for any behavioral changes for an hour and euthanized for processing. Later, the whole lungs were homogenized to quantify the uptake of nanoparticles and study the biodistribution of the PLGA-NPs and the AMNCs. Lung tissues were rinsed with PBS and fixed in 4% paraformaldehyde at 40° C. overnight and embedded with paraffin. Paraffin-embedded lungs were sectioned at 5 μm thickness and stained with hematoxylin and eosin (H&E) for histological analysis.

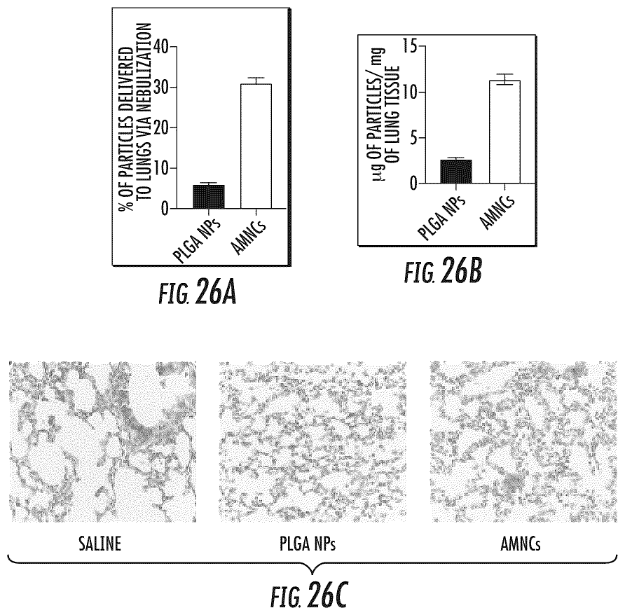

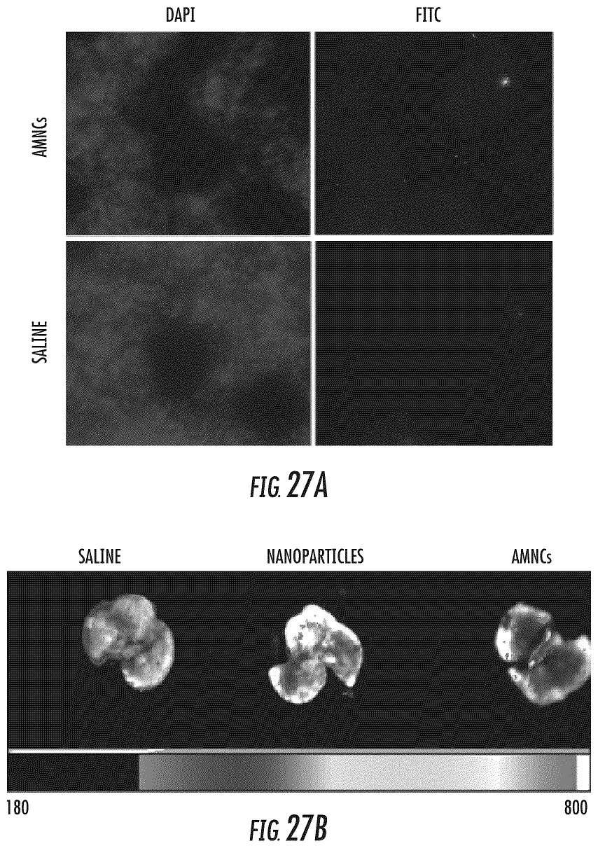

ICG-loaded nanoparticles and AMNCs were successfully delivered via inhalation as seen by the fluorescence ex vivo images of lungs (FIG. 27). Over 33% of AMNCs were delivered to the lungs via inhalation in comparison to ˜7% of PLGA-NPs over an hour after nebulization (FIG. 26A-B). Not intending to be bound by theory, the higher accumulation of AMNCs can be explained by the nanofiber's ability to improve cell uptake and traverse the mucus layer comparatively faster.

Pathological evaluation of H&E-stained lung tissue of PLGA NPs and AMNCs revealed mild to negligible changes when compared to the saline control (FIG. 26C). No collapse of alveolar space or widened or thickened alveolar septum was visible in lungs treated with AMNCs or PLGA-NPs. The H&E images also showed that mild inflammatory exudates seen in both groups were from the introduction of nanoparticles. AMNCs and PLGA-NPs show similar tissue histology, indicating that a nanofiber coating does not introduce any additional toxicities or changes, making it suitable for coating nanocarriers for pulmonary drug delivery.

### Ex Vivo Imaging

Similarly, to visually assess nanocomposite delivery in lung tissue, coumarin-6 loaded nanoparticles and AMNCs were also used to perform ex vivo imaging after animal studies using a Kodak In-Vivo Multispectral Imaging System (Carestream Health Inc., New Haven, CT). Coumarin-6 dye was used because of its superior fluorescence compared to NIR ICG dye for fluorescent microscopy purposes. Briefly, after nebulization with nanoparticles or AMNCs, the mice lungs were inflated with optimal cutting temperature compound (OCT) and dissected for further processing. Dissected lungs embedded in OCT were sectioned using a cryostat (Leica Biosystems, Germany) with a thickness of 50 μm in various regions in the lungs and stained for cell nuclei with NucBlue (ThermoFisher). Microscopic slides with processed tissues were imaged using a fluorescent microscope (ECHO, San Diego) at 40× magnification.

Fluorescent images showed that coumarin-6-loaded AMNCs were localized along with DAPI stained nucleus (FIG. 27A) and higher AMNC accumulation compared to that of the nanoparticle groups (FIG. 27B). These results indicate that AMNCs nebulized and inhaled by the mice were able to reach the alveolar region and are retained at a higher number compared to nanoparticles without any nanofiber coating. Overall, the biodistribution studies show that nanofiber coating onto nanoparticles is safe and enhances delivery of nanoparticles.

### H. Conclusion

In this Example, the antimicrobial drug vancomycin was successfully loaded into PLGA nanoparticles, and novel AMNCs were synthesized with enhanced drug delivery abilities. Herein, they were applied as a drug carrier to inhibit MRSA infection in primary lung alveolar epithelial cells. Nanofiber-coated AMNCs showed higher uptake in infected cells compared to nanoparticles lacking nanofibers, demonstrating their potential to deliver potent antimicrobials to infected cells with high cytocompatibility. AMNCs were able to inhibit intracellular MRSA either given directly in media or via nebulization with an increased potency compared with nanoparticles alone because of their higher affinity for uptake. The in vivo biodistribution of AMNCs showed a 3-fold higher accumulation in the lungs compared to nanoparticles without a nanofiber coating. Together, these characteristics indicate that the AMNCs have potential application for treating MRSA lung infections via inhalation and can also be applied towards other lung infections with their ability to load various payloads, including different antimicrobials.

### EXAMPLE 5

### Remdesivir-Loaded Nanocomposites Inhibit SARS-CoV-2 Infection In Vitro

### A. Introduction

Coronaviruses (CoV), a family of Coronaviridae, can cause significant pathological diseases such as respiratory tract infections in humans and other mammals. December 2019 marked the outbreak of the new coronavirus (SARS-CoV-2) which was first detected in Wuhan, China. The total number of Covid-19 cases worldwide reached almost 0.7 billion confirmed cases as of May 29, 2020, with the mortality count of CoV-2 reaching around 7 million globally, and the US had over 1 million deaths. Although scientists are putting in great efforts to find treatment or vaccine, as of now, there is no permanent remedy or cure for the patients suffering from CoV-2, which, in some cases, has led to death. Although the pandemic has come to an end, the emergence of a pandemic-potential virus is probable with the growing urbanization of societies and global connectivity. Currently, vaccines are the only tools to prevent the spread while the scientific community is engaged in developing treatment strategies for the diseases caused by these viruses.

Anyone, irrespective of age, can be infected with CoV-2, but its complications are of major concern for older people, people with diabetes where increased glucose levels in airway secretion significantly increase influenza virus replication, and other complications including inflammation and hypertension, which can be life-threatening. Drugs such as hydroxychloroquine, remdesivir, tocilizumab, and favilavir, among others, are currently under clinical trials with the aim of either interfering with viral replication or reducing complications in the lungs. Out of all these drugs, remdesivir and Nirmatrelvir (paxlovid) show promising results in inhibiting viral reproduction, and phase 3 clinical trials have shown a positive outcome. However, the free drugs are more vulnerable and susceptible to enzymatic degradation, opsonization by macrophages, and clearance by the immune system. In addition, the free drug is not entirely available at the target site due to its non-specificity, which leads to the requirement of multiple drug dosages. Therefore, it is necessary to develop a drug delivery system that can overcome these complications and be specific in delivering the drug at a specific site, such as inhalable drug delivery to the lungs. For that purpose, targeted drug delivery can reduce side effects in the elderly and improve treatment outcomes.

Nanotechnology has demonstrated great promise in the medical field. The major factors contributing to its popularity are increased bioavailability, enhanced drug targeting, improved drug solubility and stability, controlled drug release, and facilitated patient adherence. These properties of nanotechnology make it beneficial to effectively treat various lung diseases. The concerns associated with free drugs are addressed by using nanoparticle systems that can have sustained drug release and can efficiently deliver the drug at the target site.

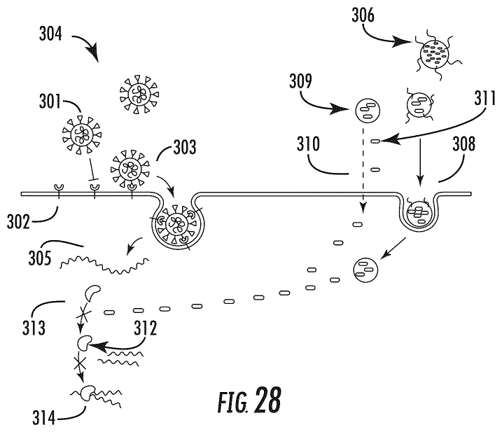

Some viral infections have been shown to reduce the cell's ability to uptake nanoparticles. Recently, technologies such as cell-penetrating peptides (CPP) have exhibited an enhanced uptake of drugs in cells via higher binding affinity to cell membranes. Towards that approach, in this example, a novel drug delivery system comprising of CPP nanofiber-coated PLGA NPs was used to deliver the antiviral drug remdesivir for the treatment of SARS-CoV-2 infections. Remdesivir-loaded PLGA nanoparticles (RDV NPs) were used as an antiviral agent against SARS-CoV-2 infections, and remdesivir-loaded nanocomposites (RDV NCs) were formulated via the coating of PLGA RDV NPs with novel supramolecular cell-penetrating peptide nanofibers to enhance cellular uptake and intracellular drug delivery. FIG. 28 is a schematic of the how the novel drug delivery system inhibits SARS-CoV-2. Coronavirus (301) enters the cytoplasm of lung epithelial cells (302) via virus binding (303) from the lung surfactant (304). Coronavirus interacts with the ribosome (305) to manufacture its proteins. CPP nanofiber-coated PLGA NPs (306) enter the lung epithelial cells into the cytoplasm from lung surfactant via cell uptake (308). Nanoparticles without nanofibers (309) enter the lung epithelial cells via passive diffusion (310). Both nanocomposites and nanoparticles can be loaded with remdesivir (311). Remdesivir is released by both the nanoparticles and nanocomposites, which inhibits RNA replication (312) by viral polymerase (313) and the formation of genomic and subgenomic RNA (314).

Herein, cellular uptake and viral load in SARS-CoV-2-infected Vero E6 cells were examined to assess the efficacy of RDV NCs in inhibiting SARS-CoV-2 infection in vitro. This embodiment of a novel drug delivery system may deliver drugs via inhalation to the lungs for the treatment of lung diseases, including lung infections such as SARS-CoV-2

### B. Methods

### Viruses

Severe acute respiratory syndrome coronavirus 2 (SARS-CoV-2) was provided by the University of Texas Medical branch. A single passage of parental viruses was propagated in Vero E6 cells (ATCC® CRL-1586™) and then collected as viral stock for this study. The viral titer of the stocks was determined by performing plaque assays in plaque forming unit (PFU) per milliliter.

### Cells

Vero E6 cells (ATCC® CRL-1586™) were maintained in Minimum Essential Medium (Gibco™ MEM, Life Technologies) containing 1% L-glutamine, 1% penicillin/streptomycin, and 10% fetal bovine serum (FBS). Alveolar type I (AT1) cells were maintained in Iscove's Modified Dulbecco's Medium (IMDM, Sigma Life Science) containing 1% L-glutamine, 1% penicillin/streptomycin, and 10% FBS.

### Synthesis of PLGA Nanoparticles

Poly(lactic-co-glycolic acid) (PLGA) nanoparticles (Blank NPs, RDV-NPs, and RDV-NCs) were synthesized via a modified single emulsion (O/W) technique as described previously.¹³ Briefly, 10 mg of remdesivir dissolved in DMSO were added to 90 mg of PLGA (copolymer ratio 50:50) in 3 mL of DCM dropwise and sonicated at 30 W for 1 min to allow dispersion of PLGA and remdesivir in the solvent. The resulting solution was added dropwise to 20 mL of filtered 5% (w/v) poly(vinyl) alcohol (PVA) solution under stirring conditions. The suspension was then sonicated at 30 watts for 2 min and then allowed to stir overnight to evaporate the organic solvent. The obtained nanoparticle suspension was centrifuged at 15,000 rpm for 30 min. The supernatant was used for the drug loading evaluation, and the PLGA NP pellet was resuspended in 3 mL of DI water and freeze-dried for 24 hours. Nanoparticles for the imaging techniques were synthesized by a similar procedure with rhodamine dyes instead of remdesivir using a double emulsion technique. Blank nanoparticles were also made similarly with no drug encapsulated in the polymer.

Nanofibers were synthesized as previously described.^{13,14} Labeled and non-labeled peptides were dissolved in tris(hydroxymethyl)-aminomethane) (Tris) buffer (pH 7.4) at 20 mM. After lyophilization of NPs, 2 mg of RDV-NPs were dissolved in Tris buffer as separate groups, and 0.5 mg of NF in suspension was added to one of the NP suspensions to prepare RDV NCs. The mixture was left to react electrostatically by rotating the solution for an hour at room temperature. Later, the sample was centrifuged at 15,000 rpm for 7 min to remove free NFs and collect the samples, which contained RDV-NCs. Rhodamine dye-loaded NPs and composites were also prepared in the same manner.

### Characterization of NF-Coated NPs or RDV NCs

### DLS Measurements

A ZETAPALS90 dynamic light scattering (DLS) detector (Brookhaven Instrument, Holtsville, NY) was used to determine the size, charge, and polydispersity of the NPs. For DLS measurements, 15 μL of 1 mg/mL NP suspension were mixed with 3 mL of DI water in a transparent cuvette and placed in the instrument to measure size, while a DLS probe was used to measure the zeta potential of the NF-coated NPs.

### Fluorescent Microscopy

Fluorescein-terminated peptides were synthesized as previously described.¹⁴ FITC-tagged peptides were mixed with rhodamine B-loaded PLGA NPs. Green color-tagged NFs were incubated with NPs loaded with rhodamine B (red color). The NCs formed were washed three times to remove any unbound NFs. Another set of NPs was similarly washed and imaged without any NFs. A fluorescent microscope (ECHO, San Diego, CA) with FITC (for NF) and Texas Red channels (For Rho B NPs) at 40× magnification was used to image the NF coating on the NPs.

### Transmission Electron Microscopy (TEM)

To generate TEM images of NF-coated NPs, 10 μL of 2 mg/mL NP suspension was added to plasma-treated Formvar Square Mesh Copper Grids and air dried after incubating with uranyl acetate for negative staining. An H-7500 TEM (Hitachi) transmission electron microscope was used to visualize the morphologies of the particles.

### Drug Loading and Drug Release Kinetics of RDV NCs

The drug/dye loading efficiency was calculated by an indirect method in which the drug present in the supernatant collected from the nanoparticle synthesis process was measured using HPLC, and the following formula was used for loading efficiency calculation:

$$\% Loading Effciency = \frac{Amount of drug used - Amount of drug in Supernatant}{Amount of drug used initially} * 100$$

A remdesivir release study was carried out for 10 days. Briefly, either 1 mg of RDV NPs or RDV NCs was taken at a concentration of 1 mg/ml and incubated at 37° C. At each predetermined time point, the samples were centrifuged at 14,000 rpm for 30 min, and supernatants were collected and stored at −20° C. for later analysis. The pellets were re-suspended in fresh PBS and incubated for further time points. Each of the drug-release aliquots was analyzed using the following HPLC method. The amount of drug released was determined against a standard curve for remdesivir.

### HPLC Method

Chromatographic analysis was performed on a liquid chromatography system (Agilent 1260) with a UV-visible detector. Remdesivir was analyzed at a flow rate of 1.2 mL/min using a mobile phase composed of 20 mM potassium dihydrogen phosphate solution and acetonitrile (50:50, v/v). Before use, the mobile phase was filtered and degassed through a 0.22 μm membrane filter. An Agilent Extend C18 (4.6 mm×250 mm, 5.0 μm particle size) column was used and operated at 25° C. Remdesivir was detected with the UV detector at 247 nm. The run time under these conditions was 10 min.

### Cytocompatibility of Nanoparticles

In this study, primary lung epithelial cells and kidney epithelial cells (Vero E6) were used to assess toxicity from NPs. NF-coated NPs were prepared as described above, in which 4000 cells/well of primary alveolar type I epithelial cells (AT1 cells) and Vero E6 cells were seeded in 96-well plates. After overnight culturing, RDV NCs were added to the cells in triplicate at various concentrations ranging from 0 to 1 mg/mL. An NF to NP ratio of 0.25 was used for the study. After 48 hours, cells were washed three times with PBS, and MTS reagent was given to the cells to assess the cell viability following the manufacturer's instructions.

### Cell Uptake of Nanoparticles

Vero E6 cells were seeded in a 24-well glass bottom plate at a density of 100,000 cells per well and incubated overnight. Cells were infected with 0.5 MOI (multiplicity of infection) SARS-CoV-2 for 2 hours. Cells were treated with rhodamine B-labeled NPs and NCs at different concentrations (0, 50, and 100 μg/mL) for 2 hours. Infected cells were fixed with 4% paraformaldehyde (PFA) for 30 minutes at room temperature (RT). The cell nuclei were stained with 4,6-diamidino-2-phenylindole (DAPI) (300 nM, Invitrogen) for 5 min at RT. The plates were observed and imaged using a Stellaris STED confocal microscope (Leica) to assess the uptake level of NPs and NCs.

### Reverse Transcriptase Quantitative Real-Time PCR (RT-qPCR)

The infected Vero E6 cells were collected in TRI Reagent, and the RNA was extracted using chloroform and isopropanol reagent. The RNA concentration was quantified using a NanoDrop spectrometer (ThermoScientific). First-strand complementary DNA (cDNA) was synthesized from the total RNA using an iSCRIPT cDNA Synthesis Kit (Bio-Rad). Then, qPCR was performed in a CFX Connect Real-Time System (Bio-Rad) using iTaq Universal Probes Supermix (Bio-Rad) for the detection of 2019-Novel Coronavirus Nucleocapsid N1 (2019-nCoV_N1) and cellular β-actin. Viral RNA copy numbers were expressed as the ratio of nCov-N1 to β-actin. Relative fold change (RFC) to the control was measured using the comparative threshold cycle ^{ΔΔ}CT method after normalizing to cellular β-actin. nCoV_N1 and cellular δ-actin gene primers and probe sequences were adapted according to previous publications.¹⁶⁻¹⁸

### Immunofluorescence Assay (IFA)

Vero E6 cells were seeded in a 24-well glass bottom plate with a concentration of 100,000 cells per well. Cells were infected with 0.5 MOI SARS-CoV-2 for 24 hours followed by various drug-loaded nanoparticle and nanocomposite treatments. Infected cells were fixed with 4% paraformaldehyde (PFA) for 30 minutes at RT. They were permeabilized with 0.1% Triton X for 20 minutes at RT and blocked in antibody dilution buffer (ADB) for 1 hour at RT. The cells were stained with a primary SARS-CoV-2 nucleocapsid monoclonal antibody (2 μg/mL, 1:500 in ADB, 200 μL per well, Invitrogen) overnight at 4° C. covered in foil and then stained with FITC-conjugated goat anti-rabbit IgG (H+L) cross-adsorbed secondary antibody (2 μg/mL, 1.3:1000 in ADB, 200 μL per well, Invitrogen) on a shaker for 1 hour at RT covered in foil. The nuclei were stained with DAPI (300 nM, Invitrogen) for 5 minutes at RT. Images of the cells were captured using a Stellaris STED confocal microscope (Leica).

### Plaque Assays

Vero E6 cells were seeded in 6-well plates at a density of 600,000 cells per well and incubated overnight. Supernatants that were collected from the pre-infection followed by drug-treated cells for the qPCR were serially diluted tenfold and used to inoculate monolayers of Vero E6 cells. After 1 hour of incubation at 37° C. with 5% CO₂, the virus inoculum was removed and covered with an overlay medium containing 1% SeaPlaque agarose (Lonza). The plates were incubated for 24 to 48 hours until plaques were formed. A plaque is a circular zone of infected cells, and each plaque represents one infectious virus. To determine the viral titer, plaques were stained with Neutral Red for 3 hours before counting. The titer of the virus was calculated in plaque forming unit per milliliter (PFU/mL) using the formula:

$$\frac{PFU}{mL} = \frac{Average number of plaques \times dilution factor}{Volume of the sample added to the plate}$$

### Statistical Analysis

All data generated were generated in replicate, if not mentioned. Data were processed using the GraphPad Prism software, and one-way ANOVA analysis was performed with multiple comparisons done using Tukey's method.

### C. Results and Discussion

### Synthesis and Characterization of Remdesivir-Loaded PLGA Nanocomposites

Remdesivir was utilized into this embodiment of the drug delivery system herein, but various other potential antiviral drugs, such as ritonavir, lopinavir, and nirmatrelvir, can also be incorporated into this drug delivery system.

Drug-loaded nanoparticles and nanocomposites were synthesized using solvent evaporation and physical adsorption methods.¹³ The prepared materials were characterized to confirm the loading of remdesivir and the coating of cell penetration nanofiber peptides onto the PLGA nanoparticles. TEM images revealed RDV NPs have an average size of about 100-150 nm with a spherical morphology (FIG. 29A). Nanofiber coating was performed as per previously established methods.¹³ Nanofiber coating of remdesivir-loaded NPs increased the size of nanoparticles from 154±18 nm to 369±48 nm. FITC-labeled nanofibers were coated onto PLGA NPs via physical adsorption exploiting the electrostatic interaction between the positively charged nanofibers and the negatively charged PLGA NP surface. Fluorescent microscopy revealed the presence of nanoparticles and nanofibers overlap in the composites (FIG. 29B). Zeta potential values of uncoated RDV NPs increased from −10 mV to +9 mV after incubation with nanofibers, indicating the presence of positively charged nanofiber coating. The results show the successful coating of nanofibers onto PLGA-NPs to make nanocomposites.

Remdesivir loading was analyzed using liquid chromatography. Drug loading efficiency was calculated via the indirect loading method and found to be ˜25%. Various nanoparticle drug release solutions were collected via dialysis at physiological conditions, including at 37° C. and pH 7.4 using PBS buffer as a sink. The drug release of remdesivir from RDV NPs showed a biphasic drug release with an initial burst release of 47% over 24 hrs, followed by a sustained release until day 10 with 55% of total drug release. Drug release profiles of remdesivir from PLGA show a burst release suitable for faster kinetics in inhibiting viral replication, which is followed by sustained release, maintaining the therapeutic levels of the drug (FIG. 29C). Overall, the nanoparticle-based drug delivery system herein can improve antiviral drug bioavailability, provide controlled drug release, and reduce side effects from multiple doses needed to maintain the therapeutic levels drug in situ.

Conventional cell membrane penetrating peptides with their cationic nature show cytotoxicity at higher concentrations. Here, it is shown that nanofiber-coated remdesivir nanoparticles show no significant cytotoxicity up to 1000 μg/mL (FIG. 29D). Not intending to be bound by theory, tolerance to high concentrations of nanocomposites in cells can be explained by the coating/physical adsorption approach, which, unlike chemical linking, has higher motility and low temporal activity, and nanofibers coated onto the nanoparticle improves the binding affinity of the nanoparticle as a whole, during uptake, leading to enhanced intracellular drug delivery.

### Nanofiber-Coated PLGA NPs Show Improved Uptake in SARS-Col′-2 Infected Vero E6 Cells

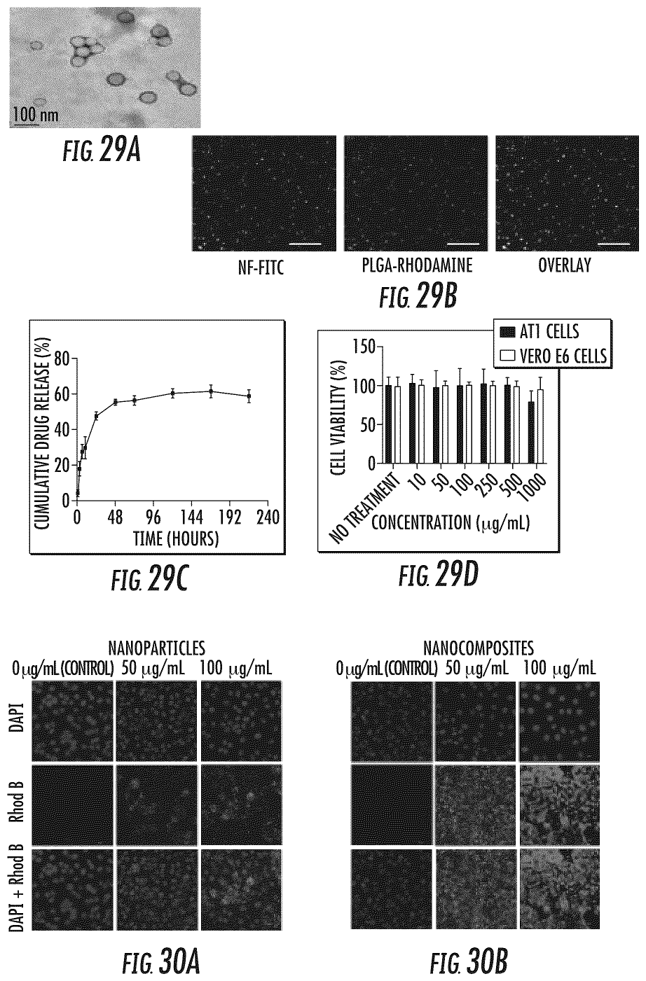

Enhancing NP uptake in cells can improve the therapeutic index of antiviral drugs by increasing drug availability intracellularly, especially in the case of inhibiting viral replication, which occurs inside the cytoplasm of the cell. PLGA-NPs with cell-penetrating nanofibers improves the uptake of NPs in primary lung cells and is suitable for pulmonary drug delivery. Accordingly, various other cell-penetrating peptides have also been employed to improve the drug delivery of antivirals, but concerns about toxicity remain. Herein, nanocomposites with better safety profiles were employed to enhance intracellular drug delivery in SARS-CoV-2-infected cells. PLGA-NPs were labeled by loading with rhodamine B to visualize their uptake in cells. Nanocomposites with nanofiber coating showed a dose-dependent increase in cellular uptake, similar to plain nanoparticles, up to 250 μg/mL of particles (FIG. 30A-B). To note, a significantly enhanced uptake of nanocomposites was observed compared to plain particles without nanofiber coating. Not intending to be bound by theory, this enhancement can be explained by the highly efficient membrane penetrating nanofiber binding activity during the cell membrane uptake of nanoparticles. The developed nanocomposites with nanofiber coating have exhibited a superior cell uptake compared to plain PLGA nanoparticles in SARS-CoV-2 infected cells. Macropinocytosis is a major endocytic pathway utilized by primary mammalian cells for the uptake of these nanocomposites. Therefore, nanocomposites with enhanced uptake in cells are a desirable drug carrier to deliver antivirals with an intracellular mode of action, such as inhibition of viral replication, especially among pulmonary pathologies.

### Remdesivir-Loaded Nanocomposites Inhibit SARS-CoV-2 Infection In Vitro

Remdesivir, which binds to the viral RNA-dependent RNA polymerase, has been reported to be effective against SARS-CoV-2 infection both in vivo and in vitro.²⁶ However, the instability and lack of specificity of free remdesivir within the body pose challenges in achieving an efficient treatment at lower concentrations, and the requirement of multiple dosages often leads to varying degrees of side effects experienced by patients. Nanotechnology has emerged as a highly promising technology for effectively addressing viral detection, prevention, and treatment. Biocompatible nanoparticle platforms, such as polymer- and lipid-based nanocarriers with drugs offer better stability, release, uptake, and bioavailability. PLGA polymer-based nanoparticles have demonstrated their utility in developing anti-viral drug delivery systems.

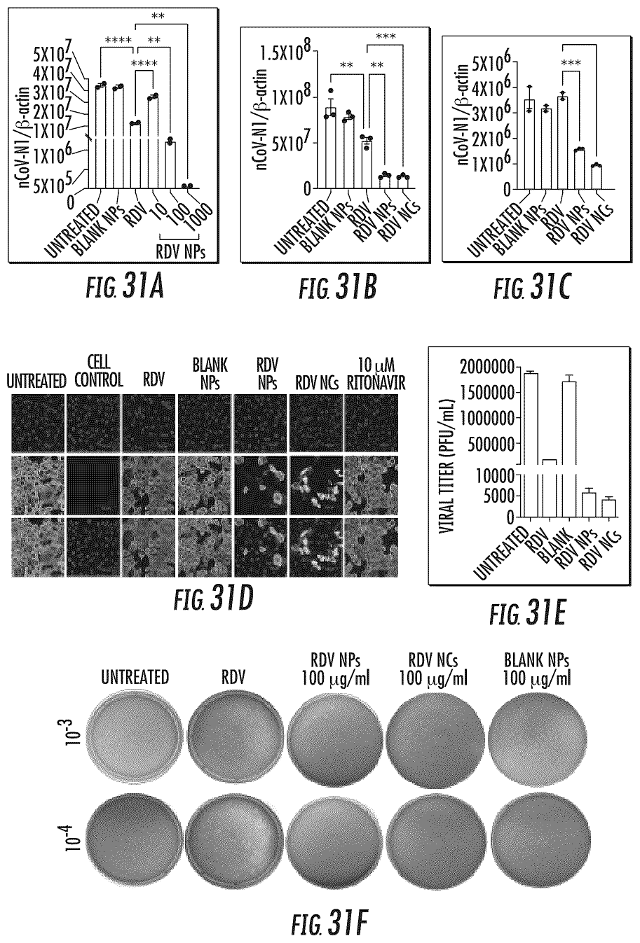

In this Example, the antiviral activity of RDV NPs was evaluated in Vero E6 cells. Vero E6 cells were infected with SARS-CoV-2 and treated with the RDV NPs and RDV NCs at various concentrations (10, 100, and 1000 μg/mL). RT-PCR data with nCoV-N11β-actin gene analysis showed a reduction in viral load above 10 g/mL RDV NPs, while 100 and 1000 μg/mL RDV NPs exhibited approximately 5- and 70-fold higher antiviral activity, respectively, compared to 200 nM remdesivir (FIG. 31A). To note, 100 μg/mL RDV NPs had an equivalent drug concentration to 200 nM remdesivir drug, and 100 μg/mL RDV NPs showed 5 times higher inhibition than 200 nM remdesivir. A time-dependent study was also performed to evaluate the antiviral efficacy of RDV NPs and RDV NCs. An enhanced reduction of viral load in RDV NCs- and RDV NPs-treated cells was observed compared to those cells treated with the free remdesivir drug and untreated control. After 48 hours, remdesivir alone did not show any significant effect in viral reduction, plausibly because of the degradation or reduced uptake in cells, while NCs and NPs loaded with remdesivir showed increased viral inhibition (FIG. 31B-C). In the drug release profiles, at 24 hours, there was a drastic inhibition of virus compared to 48 hours, in agreement with the burst drug release profile of nanoparticles. Additionally, nanocomposites showed higher viral inhibition compared to nanoparticles after 48 hours, indicating higher bioavailability of antiviral via increased uptake ability of nanocomposites by SARS-CoV-2-infected Vero E6 cells. This novel nanofiber-based delivery approach exhibits promise in reducing the transmission of virus and providing effective inhibition of SARS-CoV-2 at different levels of pathogenesis.

To visualize the inhibition of SARS-CoV-2, an immunofluorescence assay was performed in accordance with protocols outlined previously to detect SARS-CoV-2 nucleocapsid proteins. Nucleocapsid antibody staining of SARS-CoV-2 infected Vero E6 cells enabled visualizing the viral load in various treatment groups. Clearly, both RDV NPs and RDV NCs showed more than 80% viral inhibition without inducing cytotoxicity, as seen by nuclei staining (FIG. 31D). The immunofluorescence study showed the ability of RDV NCs to successfully deliver remdesivir without degradation in the cytoplasm, hence improving its biological activity in inhibiting viral replication intracellularly.

The results of RT-qPCR and IFA were further validated by viral plaque assays. Plaque assays are considered one of the most precise methods for the direct quantification of viruses. The plaque assay was performed to estimate the viral titer from the supernatants that were collected after 24 hours of drug treatment in Vero E6 cells pre-infected with SARS-CoV-2. The viral titer from different wells was calculated in PFU/mL, and differences in viral titer were compared to the untreated virus control (FIG. 31E). The viral titer in RDV NCs- and RDV NPs-treated supernatants showed approximately 463- and 322-fold reduction, respectively, compared to the untreated control. The plaque assay showed a reduced number of plaques for RDV NPs- and RDV NCs-treated cells, which indicated an increased level of inhibition of SARS-CoV-2 compared to the untreated and RDV control (FIG. 31F). The inhibition pattern of SARS-CoV-2 found in plaque assays confirmed the results of RT-qPCR and IFA. Thus, not intending to be bound by theory, the novel nanocomposites improve the bioavailability of antivirals via enhanced intracellular delivery and enhance the therapeutic efficacy of drugs.

### D. Conclusion

In this Example, nanofiber coating onto PLGA NPs showed their improved uptake in SARS-CoV-2-infected Vero E6 cells. Remdesivir-loaded nanoparticles showed a sustained drug release of remdesivir drug in physiological conditions. The results of RT-qPCR, IFA, and plaque assays showed significant SARS-CoV-2 inhibition with nanocomposites compared to uncoated nanoparticles, indicating their superior ability for intracellular drug delivery and as a drug carrier for anti-viral therapy in pulmonary infections. These nanocomposites can be applied as an inhalable drug delivery system with their described size and drug release, which are beneficial for pulmonary infections.

### E. References

(1) Organization, W. H. COVID-19 statistics worldwide. 2020. https://covid19.who.int/(accessed 2020 May 27).
(2) Guo, W.; Li, M.; Dong, Y.; Zhou, H.; Zhang, Z.; Tian, C.; Qin, R.; Wang, H.; Shen, Y.; Du, K.; et al. Diabetes is a risk factor for the progression and prognosis of COVID-19. Diabetes Metabolism Research and Reviews 2020, n a (n/a), e3319. DOI: 10.1002/dmrr.3319 (acccessed 2020 May 27).
(3) Cristelo, C.; Azevedo, C.; Moreira Marques, J.; Nunes, R.; Sarmento, B. SARS-CoV-2 and Diabetes: New Challenges for the Disease. (1872-8227 (Electronic)). From 2020 May 21.
(4) Hulme, K. D.; Gallo, L. A.; Short, K. R. Influenza Virus and Glycemic Variability in Diabetes: A Killer Combination? Frontiers in microbiology 2017, 8, 861-861. DOI: 10.3389/fmicb.2017.00861 PubMed.
(5) Liu, C.; Zhou, Q.; Li, Y.; Garner, L. V.; Watkins, S. P.; Carter, L. J.; Smoot, J.; Gregg, A. C.; Daniels, A. D.; Jervey, S.; et al. Research and Development on Therapeutic Agents and Vaccines for COVID-19 and Related Human Coronavirus Diseases. ACS Central Science 2020, 6 (3), 315-331. DOI: 10.1021/acscentsci.0c00272.
(6) Gilead Announces Results From Phase 3 Trial of Investigational Antiviral Remdesivir in Patients with Severe COVID-19. (Foster City), 2020. (accessed Acessed April 29).
(7) Doroudian, M.; MacLoughlin, R.; Poynton, F.; Prina-Mello, A.; Donnelly, S. C. Nanotechnology based therapeutics for lung disease. Thorax 2019, 74 (10), 965. DOI: 10.1136/thoraxjnl-2019-213037.
(8) Jurek, S. C.; Hirano-Kobayashi, M.; Chiang, H.; Kohane, D. S.; Matthews, B. D. Prevention of ventilator-induced lung edema by inhalation of nanoparticles releasing ruthenium red. American journal of respiratory cell and molecular biology 2014, 50 (6), 1107-1117. DOI: 10.1165/rcmb.2013-0163OC PubMed.
(9) Zhang, C. Y.; Lin, W.; Gao, J.; Shi, X.; Davaritouchaee, M.; Nielsen, A. E.; Mancini, R. J.; Wang, Z. pH-Responsive Nanoparticles Targeted to Lungs for Improved Therapy of Acute Lung Inflammation/Injury. ACS applied materials & interfaces 2019, 11 (18), 16380-16390. DOI: 10.1021/acsami.9b04051 PubMed.
(10) D'Almeida, A. P. L.; Pacheco de Oliveira, M. T.; de Souza É, T.; de Sá Coutinho, D.; Ciambarella, B. T.; Gomes, C. R.; Terroso, T.; Guterres, S. S.; Pohlmann, A. R.; Silva, P. M.; et al. α-bisabolol-loaded lipid-core nanocapsules reduce lipopolysaccharide-induced pulmonary inflammation in mice. (1178-2013 (Electronic)). From 2017.
(11) Nguyen, H. X. Targeted Delivery of Surface-Modified Nanoparticles: Modulation of Inflammation for Acute Lung Injury. Surface Modification of Nanoparticles for Targeted Drug Delivery 2019, 331-353. DOI: 10.1007/978-3-030-06115-9 17 PMC.
(12) Abo-Zeid, Y.; Williams, G. R.; Touabi, L.; McLean, G. R. An investigation of rhinovirus infection on cellular uptake of poly(glycerol-adipate) nanoparticles. Int J Pharm 2020, 589, 119826. DOI: 10.1016/j.ijpharm.2020.119826.
(13) Chintapula, U.; Yang, S.; Nguyen, T.; Li, Y.; Jaworski, J.; Dong, H.; Nguyen, K. T. Supramolecular Peptide Nanofiber/PLGA Nanocomposites for Enhancing Pulmonary Drug Delivery. ACS Appl Mater Interfaces 2022, 14 (51), 56498-56509. DOI: 10.1021/acsami.2c15204.
(14) Dong, S. Y. a. H. Modular design and self-assembly of multidomain peptides towards cytocompatible supramolecular cell penetrating nanofibers. RSC Adv., 2020; Vol. 10, p 29469
(15) Anantharajah, A.; Helaers, R.; Defour, J. P.; Olive, N.; Kabera, F.; Croonen, L.; Deldime, F.; Vaerman, J. L.; Barbee, C.; Bodeus, M.; et al. How to choose the right real-time RT-PCR primer sets for the SARS-CoV-2 genome detection? J Virol Methods 2021, 295, 114197. DOI: 10.1016/j.jviromet.2021.114197.
(16) Bai, F.; Wang, T.; Pal, U.; Bao, F.; Gould, L. H.; Fikrig, E. Use of RNA interference to prevent lethal murine west nile virus infection. J Infect Dis 2005, 191 (7), 1148-1154. DOI: 10.1086/428507.
(17) Bai, F.; Kong, K.-F.; Dai, J.; Qian, F.; Zhang, L.; Brown, C. R.; Fikrig, E.; Montgometry, R. R. A Paradoxical Role for Neutrophils in the Pathogenesis of West Nile Virus. The Journal of Infectious Diseases 2010, 202 (12), 1804-1812. DOI: 10.1086/657416 (acccessed Jun. 1, 2023).
(18) Paul, A. M.; Acharya, D.; Duty, L.; Thompson, E. A.; Le, L.; Stokic, D. S.; Leis, A. A.; Bai, F. Osteopontin facilitates West Nile virus neuroinvasion via neutrophil “Trojan horse” transport. Sci Rep 2017, 7 (1), 4722. DOI: 10.1038/s41598-017-04839-7.
(19) Marzi, M.; Vakil, M. K.; Bahmanyar, M.; Zarenezhad, E. Paxlovid: Mechanism of Action, Synthesis, and. Biomed Res Int 2022, 2022, 7341493. DOI: 10.1155/2022/7341493.
(20) Mousavi Maleki, M. S.; Sardari, S.; Ghandehari Alavijeh, A.; Madanchi, H. Recent Patents and FDA-Approved Drugs Based on Antiviral Peptides and Other Peptide-Related Antivirals. Int J Pept Res Ther 2023, 29 (1), 5. DOI: 10.1007/s¹⁰⁹⁸⁹-022-10477-z.
(21) Ucar, B.; Acar, T.; Arayici, P. P.; Derman, S. A nanotechnological approach in the current therapy of COVID-19: model drug oseltamivir-phosphate loaded PLGA nanoparticles targeted with spike protein binder peptide of SARS-CoV-2. Nanotechnology 2021, 32 (48). DOI: 10.1088/1361-6528/ac1c22.
(22) Yang, H.; Li, J.; Patel, S. K.; Palmer, K. E.; Devlin, B.; Rohan, L. C. Design of Poly(lactic-. Pharmaceutics 2019, 11 (4). DOI: 10.3390/pharmaceutics11040184.
(23) Derakhshankhah, H.; Jafari, S. Cell penetrating peptides: A concise review with emphasis on biomedical applications. Biomed Pharmacother 2018, 108, 1090-1096. DOI: 10.1016/j.biopha.2018.09.097.
(24) Xie, J.; Bi, Y.; Zhang, H.; Dong, S.; Teng, L.; Lee, R. J.; Yang, Z. Cell-Penetrating Peptides in Diagnosis and Treatment of Human Diseases: From Preclinical Research to Clinical Application. Front Pharmacol 2020, 11, 697. DOI: 10.3389/fphar.2020.00697.
(25) Sadeghian, I.; Heidari, R.; Sadeghian, S.; Raee, M. J.; Negahdaripour, M. Potential of cell-penetrating peptides (CPPs) in delivery of antiviral therapeutics and vaccines. Eur J Pharm Sci 2022, 169, 106094. DOI: 10.1016/j.ejps.2021.106094.
(26) Wang, M.; Cao, R.; Zhang, L.; Yang, X.; Liu, J.; Xu, M.; Shi, Z.; Hu, Z.; Zhong, W.; Xiao, G. Remdesivir and chloroquine effectively inhibit the recently emerged novel coronavirus (2019-nCOV) in vitro. Cell Res 2020, 30 (3), 269-271. DOI: 10.1038/s⁴¹⁴²²-020-0282-0.
(27) Remdesivir (Veklury) [package insert]. Food and Drug Administration. https://www.accessdata.fda.gov/drugsatfda_docs/label/2022/214787Orig1s015lbl.pdf (accessed.
(28) Gandhi, S.; Klein, J.; Robertson, A. J.; Peña-Hernández, M. A.; Lin, M. J.; Roychoudhury, P.; Lu, P.; Fournier, J.; Ferguson, D.; Mohamed Bakhash, S. A. K.; et al. De novo emergence of a remdesivir resistance mutation during treatment of persistent SARS-CoV-2 infection in an immunocompromised patient: a case report. Nat Commun 2022, 13 (1), 1547. DOI: 10.1038/s41467-022-29104-y.
(29) Cheng, M.; Fowler, R.; Murthy, S.; Pinto, R.; Sheehan, N. L.; Tseng, A. Remdesivir in Patients With Severe Kidney Dysfunction: A Secondary Analysis of the CATCO Randomized Trial. JAMA Netw Open 2022, 5 (8), e2229236. DOI: 10.1001/jamanetworkopen.2022.29236.
(30) Puri, A.; Loomis, K.; Smith, B.; Lee, J. H.; Yavlovich, A.; Heldman, E.; Blumenthal, R. Lipid-based nanoparticles as pharmaceutical drug carriers: from concepts to clinic. Crit Rev Ther Drug Carrier Syst 2009, 26 (6), 523-580. DOI: 10.1615/critrevtherdrugcarriersyst.v26.16.10.
(31) Mandal, S.; Belshan, M.; Holec, A.; Zhou, Y.; Destache, C. J. An Enhanced Emtricitabine-Loaded Long-Acting Nanoformulation for Prevention or Treatment of HIV Infection. Antimicrob Agents Chemother 2017, 61 (1). DOI: 10.1128/AAC.01475-16.
(32) Mandal, S.; Prathipati, P. K.; Belshan, M.; Destache, C. J. A potential long-acting bictegravir loaded nano-drug delivery system for HIV-1 infection: A proof-of-concept study. Antiviral Res 2019, 167, 83-88. DOI: 10.1016/j.antiviral.2019.04.007.
(33) Hathout, R. M.; Abdelhamid, S. G.; Metwally, A. A. Chloroquine and hydroxychloroquine for combating COVID-19: Investigating efficacy and hypothesizing new formulations using Bio/chemoinformatics tools. Inform Med Unlocked 2020, 21, 100446. DOI: 10.1016/j.imu.2020.100446.
(34) Karmacharya, M.; Kumar, S.; Gulenko, O.; Cho, Y. K. Advances in Facemasks during the COVID-19 Pandemic Era. ACS Appl Bio Mater 2021, 4 (5), 3891-3908. DOI: 10.1021/acsabm.0c01329.
(35) Emam, M. H.; Elezaby, R. S.; Swidan, S. A.; Loutfy, S. A.; Hathout, R. M. Cerium Oxide Nanoparticles/Polyacrylonitrile Nanofibers as Impervious Barrier against Viral Infections. Pharmaceutics 2023, 15 (5). DOI: 10.3390/pharmaceutics 15051494.
(36) Cai, X.; Chen, M.; Prominski, A.; Lin, Y.; Ankenbruck, N.; Rosenberg, J.; Nguyen, M.; Shi, J.; Tomatsidou, A.; Randall, G.; et al. A Multifunctional Neutralizing Antibody-Conjugated Nanoparticle Inhibits and Inactivates SARS-CoV-2. Adv Sci (Weinh) 2022, 9 (2), e2103240. DOI: 10.1002/advs.202103240.
(37) Ejsmont, A.; Warowicka, A.; Broniarczyk, J.; Goscianska, J. The synergistic effect of Cu-MOF nanoparticles and immunomodulatory agent on SARS-CoV-2 inhibition. Chem Commun (Camb) 2023, 59 (33), 4907-4910. DOI: 10.1039/d3cc00894k.
(38) Sanna, V.; Satta, S.; Hsiai, T.; Sechi, M. Development of targeted nanoparticles loaded with antiviral drugs for SARS-CoV-2 inhibition. Eur J Med Chem 2022, 231, 114121. DOI: 10.1016/j.ejmech.2022.114121.
(39) Mohammadi, M.; Dehghani, P.; Mohseninia, A.; Roozbehani, M.; Hemphill, A.; Hesamizadeh, K. Incorporation of the Tat cell-penetrating peptide into nanofibers improves the respective antitumor immune response. J Cell Physiol 2021, 236 (2), 1401-1417. DOI: 10.1002/jcp.29946.
(40) Emam, M. H.; Nageh, H.; Ali, F.; Taha, M.; ElShehaby, H. A.; Amin, R.; Kamoun, E. A.; Loutfy, S. A.; Kasry, A. Inhibition of SARS-CoV-2 spike protein entry using biologically modified polyacrylonitrile nanofibers: RSC Adv 2022, 12 (25), 16184-16193. DOI: 10.1039/d2ra01321e.
(41) Zhu, A. K.; Li, S. S.; Yu, S. P.; Zhang, Z. Y.; Li, H.; Li, J. F.; Gao, S. S.; Chen, H.; Zhao, J.; Shi, R.; et al. A pair of SARS-CoV-2 nucleocapsid protein monoclonal antibodies shows high specificity and sensitivity for diagnosis. Virol Sin 2022, 37 (6), 942-945. DOI: 10.1016/j.virs.2022.10.003.
(42) Mendoza, E. J.; Manguiat, K.; Wood, H.; Drebot, M. Two Detailed Plaque Assay Protocols for the Quantification of Infectious SARS-CoV-2. Curr Protoc Microbiol 2020, 57 (1), ecpmc105. DOI: 10.1002/cpmc. 105.
(43) Neupane, B.; Bai, F. Quantification of West Nile Virus by Plaque-Forming Assay. Methods Mol Biol 2023, 2585, 9-14. DOI: 10.1007/978-1-0716-2760-0 2.
(44) Karim, S. U.; Bai, F. Introduction to West Nile Virus. Methods Mol Biol 2023, 2585, 1-7. DOI: 10.1007/978-1-0716-2760-0 1.
(45) Nazneen, F.; Thompson, E. A.; Blackwell, C.; Bai, J. S.; Huang, F.; Bai, F. An effective live-attenuated Zika vaccine candidate with a modified 5′ untranslated region. NPJ Vaccines 2023, 8 (1), 50. DOI: 10.1038/s41541-023-00650-w.

### ADDITIONAL EMBODIMENTS

Some additional, non-limiting, example embodiments are provided below.

Embodiment 1. A composition comprising:
a nanoparticle;
a plurality of nanofibers disposed on an exterior surface of the nanoparticle; and
a payload disposed within an interior of the nanoparticle, wherein the nanoparticle has an average size in three dimensions and an average surface area;
wherein the plurality of nanofibers has an average length in a long dimension; and
wherein the ratio of the average surface area of the nanoparticle to the average length of the nanofibers, in units of (μm), is between 0.6 and 4,000, and/or wherein a ratio of the average size of the nanoparticle to the average length of the nanofibers is between 2 and 250.

Embodiment 2. The composition of Embodiment 1, wherein the ratio of the average size of the nanoparticle to the average length of the nanofibers is between 5 and 100.

Embodiment 3. The composition of Embodiment 1, wherein the ratio of the average size of the nanoparticle to the average length of the nanofibers is between 5 and 30.

Embodiment 4. The composition of Embodiment 1, wherein the average size of the nanoparticle in three dimensions is between 0.1 μm and 5 μm, between 0.2 μm and 5 μm, or between 0.2 μm and 2 μm.

Embodiment 5. The composition of any of the preceding Embodiments, wherein the average length of the nanofibers in the long dimension is between 20 nm and 50 nm.

Embodiment 6. The composition of any of the preceding Embodiments, wherein the average width of the nanofibers in one or two dimensions is less than 10 nm.

Embodiment 7. The composition of any of the preceding Embodiments, wherein the nanofibers are present in the composition in an amount of 0.5 to 15 wt. %, based on the total weight of the composition.

Embodiment 8. The composition of any of the preceding Embodiments, wherein the exterior surface of the nanoparticle has an opposite charge compared to a solvent-facing charge density of the plurality of nanofibers.

Embodiment 9. The composition of any of the preceding Embodiments, wherein the exterior surface of the nanoparticle is negatively charged or has a negative zeta potential.

Embodiment 10. The composition of the any of the preceding Embodiments, wherein the nanofibers have a positive solvent-facing charge density or a positive zeta potential.

Embodiment 11. The composition of any of the preceding Embodiments, wherein the nanoparticle is formed from a biocompatible and/or biodegradable material.

Embodiment 12. The composition of any of the preceding Embodiments, wherein the nanoparticle comprises a lipid nanoparticle or a liposome.

Embodiment 13. The composition of any of the preceding Embodiments, wherein the nanoparticle is formed from an inorganic material.

Embodiment 14. The composition of Embodiment 13, wherein the nanoparticle is formed from a ceramic material, a mixture or combination of ceramic materials, a bioglass, a metal, a mixture, combination, or alloy of metals, or a combination of two or more of the foregoing.

Embodiment 15. The composition of Embodiment 14, wherein the nanoparticle is formed from SiO₂, TiO₂, ZrO₂, CaO, MgO, Na₂O, K₂O, P₂O₅, hydroxyapatite (Ca₁₀(PO₄)₆(OH)₂), stainless steel, a cobalt-chromium alloy, titanium, a titanium alloy, a silicone, or a combination of two or more of the foregoing.

Embodiment 16. The composition of any of Embodiments 1-12, wherein the nanoparticle is formed from an organic material.

Embodiment 17. The composition of Embodiment 16, wherein the nanoparticle is formed from a polymer.

Embodiment 18. The composition of Embodiment 16, wherein the nanoparticle is formed from a polyvinylchloride (PVC), a polyethylene (PE), a polypropylene (PP), a polytetrafluoroethylene (PTFE), a polymethylmethacrylate (PMMA), a poly(trimethylene carbonate) (PTMC), a poly(lactic-co-glycolic acid) (PLGA), a poly(lactic acid) (PLA), a poly(glycolic acid) (PGA), a polysaccharide, or a combination or mixture of two or more of the foregoing.

Embodiment 19. The composition of any of the preceding Embodiments, wherein the nanoparticle is porous.

Embodiment 20. The composition of any of the preceding Embodiments, wherein the nanofibers comprise polypeptide nanofibers.

Embodiment 21. The composition of Embodiment 20, wherein the polypeptide nanofibers comprise 15 to 40, 20 to 40, 20 to 35, 21 to 40, 21 to 35, or 21 to 32 residues per peptide chain.

Embodiment 22. The composition of Embodiment 20, wherein the nanofibers comprise self-assembled polypeptide nanofibers.

Embodiment 23. The composition of Embodiment 20, wherein the nanofibers comprise multidomain peptides (MDPs).

Embodiment 24. The composition of Embodiment 20, wherein the nanofibers have a peptide sequence of Kₓ(QW)₆E_{y}, where x is an integer ranging from 8 to 15 and y is an integer ranging from 1 to 5, or x is an integer ranging from 8 to 10 and y is an integer ranging from 1 to 3 (SEQ ID NO: 4).

Embodiment 25. The composition of Embodiment 24, wherein the nanofibers have a peptide sequence of K₁₀(QW)₆E₃ (SEQ ID NO: 1).

Embodiment 26. The composition of any of the preceding Embodiments, wherein the payload comprises an imaging agent, a therapeutic agent, a theranostic agent, or a combination of two or more of the foregoing.

Embodiment 27. The composition of any of the preceding Embodiments, wherein the payload is physically entrapped within the interior of the nanoparticle.

Embodiment 28. The composition of any of the preceding Embodiments, wherein the payload is operable to diffuse out of the interior of the nanoparticle when the composition is disposed in an aqueous or biological environment.

Embodiment 29. The composition of any of the preceding Embodiments, wherein the payload is present in the composition in an amount of 1-80 wt. %, based on the total weight of the composition.

Embodiment 30. The composition of any of the preceding Embodiments, wherein the payload comprises nucleic acids, proteins, peptides, chemotherapeutics, vaccine components, antibiotics, or a combination of two or more of the foregoing.

Embodiment 31. A method of treating and/or diagnosing a condition or disease in a patient in need thereof, the method comprising:
disposing the composition of any of Embodiments 1-30 within a biological compartment of the patient.

Embodiment 32. The method of Embodiment 31 further comprising:
penetrating a membrane of a cell or population of cells within the biological compartment with the plurality of nanofibers of the composition.

Embodiment 33. The method of Embodiment 32 further comprising:
releasing at least a portion of the payload of the composition within a cytosol of the cell or population of cells after penetrating the membrane of the cell or population of cells.

Embodiment 34. The method of Embodiment 33 further comprising:
biologically degrading the nanoparticle and/or the plurality of nanofibers of the composition after penetrating the membrane of the cell or population of cells.

Embodiment 35. The method of Embodiment 33, wherein:
the payload comprises an imaging agent or a theranostic agent; and
the method further comprises imaging the cell or population of cells with the imaging agent or theranostic agent.

Embodiment 36. The method of any of Embodiments 31-35, wherein the composition is disposed within the biological compartment of the patient by inhalation or nebulization.

Embodiment 37. The method of any of Embodiments 31-36, wherein:
the condition or disease comprises a respiratory condition or disease; and
the biological compartment is a pulmonary site.

Embodiment 38. The method of Embodiment 37, wherein the respiratory condition or disease comprises a degenerative or genetic disease.

Embodiment 39. The method of Embodiment 37, wherein the respiratory condition or disease comprises idiopathic lung fibrosis, a chronic obstructive pulmonary disease (COPD), or a lung cancer.

Embodiment 40. The method of Embodiment 37, wherein:
the respiratory condition or disease is caused by a pathogen or product of a pathogen; and
the payload comprises a therapeutic agent effective for the treatment of the condition or disease caused by the pathogen or product of the pathogen.

Embodiment 41. The method of Embodiment 40, wherein the pathogen or product of the pathogen comprises one or more of Methicillin-Resistant Staphylococcus Aureus (MRSA), Alpha-toxin (Hla), Staphylococcal protein A (Spa), and SARS-CoV-2.

Embodiment 42. The method of Embodiment 40, wherein:
the respiratory condition or disease comprises Mycobacterium tuberculosis and/or Streptococcus pneumonia; and
the pathogen or product of the pathogen comprises mycobacterium and/or streptococcus bacterium.

All patent documents referred to herein are incorporated by reference in their entireties. Various embodiments of the invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

1. A composition comprising:
a nanoparticle;
a plurality of nanofibers disposed on an exterior surface of the nanoparticle; and
a payload disposed within an interior of the nanoparticle,
wherein the nanoparticle has an average size in three dimensions;
wherein the plurality of nanofibers has an average length in a long dimension; and
wherein a ratio of the average size of the nanoparticle to the average length of the nanofibers is between 2 and 250.

2. The composition of claim 1, wherein the ratio of the average size of the nanoparticle to the average length of the nanofibers is between 5 and 100.

3. The composition of claim 3, wherein the ratio of the average size of the nanoparticle to the average length of the nanofibers is between 5 and 30.

4. The composition of claim 1, wherein the plurality of nanofibers has an average length in a long dimension;
and wherein the ratio of the average surface area of the nanoparticle to the average length of the nanofibers, in units of (μm), is between 0.6 and 4,000.

5. The composition of claim 1, wherein the average size of the nanoparticle in three dimensions is between 0.1 μm and 5 μm.

6. The composition of claim 1, wherein the average length of the nanofibers in the long dimension is between 20 nm and 50 nm.

7. The composition of claim 1, wherein the average width of the nanofibers in one or two dimensions is less than 10 nm.

8. The composition of claim 1, wherein the nanofibers are present in the composition in an amount of 0.5 to 15 wt. %, based on the total weight of the composition.

9. The composition of claim 1, wherein the exterior surface of the nanoparticle has an opposite charge compared to a solvent-facing charge density of the plurality of nanofibers.

10. The composition of claim 1, wherein the exterior surface of the nanoparticle is negatively charged or has a negative zeta potential.

11. The composition of claim 1, wherein the nanofibers have a positive solvent-facing charge density or a positive zeta potential.

12. The composition of claim 1, wherein the nanoparticle is formed from a biocompatible and/or biodegradable material.

13. The composition of claim 1, wherein the nanoparticle comprises a lipid nanoparticle or a liposome.

14. The composition of claim 1, wherein the nanoparticle is formed from an inorganic material.

15. (canceled)

16. (canceled)

17. The composition of claim 1, wherein the nanoparticle is formed from an organic material.

18. (canceled)

19. (canceled)

20. (canceled)

21. The composition of claim 1, wherein the nanofibers comprise polypeptide nanofibers.

22. The composition of claim 21, wherein the polypeptide nanofibers comprise 15 to 40 residues per peptide chain.

23. The composition of claim 21, wherein the nanofibers comprise self-assembled polypeptide nanofibers.

24. The composition of claim 21, wherein the nanofibers comprise multidomain peptides (MDPs).

25. The composition of claim 21, wherein the nanofibers have a peptide sequence of Kₓ(QW)₆E_{y}, where x is an integer ranging from 8 to 15 and y is an integer ranging from 1 to 5 (SEQ ID NO: 4).

26. The composition of claim 25, wherein the nanofibers have a peptide sequence of K₁₀(QW)₆E₃ (SEQ ID NO: 1).

27. The composition of claim 1, wherein the payload comprises an imaging agent, a therapeutic agent, a theranostic agent, or a combination of two or more of the foregoing.

28. The composition of claim 1, wherein the payload is physically entrapped within the interior of the nanoparticle.

29. The composition of claim 1, wherein the payload is operable to diffuse out of the interior of the nanoparticle when the composition is disposed in an aqueous or biological environment.

30. The composition of claim 1, wherein the payload is present in the composition in an amount of 1-80 wt. %, based on the total weight of the composition.

31. The composition of claim 1, wherein the payload comprises nucleic acids, proteins, peptides, chemotherapeutics, vaccine components, antibiotics, or a combination of two or more of the foregoing.

32-43. (canceled)
